(19) United States
(12) Patent Application Publication
(10) Pub. No.: US 2024/0299345 A1
(43) Pub. Date: Sep. 12, 2024

(54) GINKGOLIDE B AND ITS DERIVATIVES FOR INHIBITING AND/OR REVERSING AGING

(71) Applicant: CHINA MEDICAL UNIVERSITY, Taichung (TW)

(72) Inventors: Oscar Kuang-Sheng LEE, Taichung (TW); Chien-Wei LEE, Taichung (TW); Yu-Fan CHEN, Taichung (TW); Hao-Hsiang WU, Taichung (TW)

(21) Appl. No.: 18/583,080

(22) Filed: Feb. 21, 2024

Related U.S. Application Data
(60) Provisional application No. 63/488,415, filed on Mar. 3, 2023.

(51) Int. Cl. A61K 31/365; A61P 39/00

(52) U.S. Cl. CPC A61K 31/365; A61P 39/00

(57) ABSTRACT

The present disclosure relates to use of a pharmaceutical composition in the manufacture of a medicament for inhibiting and/or reversing aging in a subject. The pharmaceutical composition comprises an effective amount of ginkgolide B (GB) or a derivative thereof.

### PRIORITY DATA

This application claims benefit of and priority to U.S. Provisional Patent Application No. 63/488,415, filed Mar. 3, 2023, the contents of which is incorporated by reference in its entirety.

### FIELD OF THE INVENTION

The present disclosure relates to a field of inhibiting and/or reversing aging. Particularly, the present disclosure relates to ginkgolide B (GB) and its derivatives for use in this field.

### BACKGROUND OF THE INVENTION

Lifestyle intervention regimes, including exercise, calorie restriction, and intermittent fasting, have been proved to successfully enhance healthspan or lifespan. However, the inconsistent findings, due to different ages and ethnicities, failures in long-term compliance, and variable responses, limit their application. Based on advanced genetic approaches, the identification of aging-related conserved pathways has spawned pharmacologic interventions, such as aspirin, 17α-oestradiol, acarbose, nor dihydroguaiaretic acid, which target specific genes or pathways to improve lifespan and healthspan in model organisms. However, most aforementioned interventions are failed to extend lifespan upon used in females. Recently, natural small molecules, such as curcumin, resveratrol, rapamycin, quercetin and procyanidin C1, are emerging as a promising strategy to increase longevity and healthspan in diverse model organisms. However, many of them have not demonstrated a statistically significant effect on lifespan in multicenter mouse trials. Chronic use of rapamycin, which is recognized to promote longevity in most experimental models, does not improve health status and induces adverse effects, such as insulin resistance and cataracts, due to off-target effects. Hence, it is imperative to develop a useful and accessible therapeutic approach to improve healthspan and lifespan.

### SUMMARY OF THE INVENTION

In one aspect, the present disclosure provides a method for inhibiting and/or reversing aging in a subject, comprising administering of ginkgolide B (GB) or a derivative thereof to the subject.

In another aspect, the present disclosure provides a method for inhibiting and/or reversing aging in a subject, comprising administering a composition to the subject, wherein the composition comprises an effective amount of ginkgolide B or a derivative thereof.

In another aspect, the present disclosure provides a use of a composition in the manufacture of a preparation for inhibiting and/or reversing aging in a subject, wherein the composition comprises an effective amount of ginkgolide B or a derivative thereof.

In one embodiment, the method as described herein is further for extending healthspan in pathological and steady-state conditions.

In one embodiment, the method as described herein is further for extending lifespan.

In some embodiments, the method as described herein is further for one of more of the following: enhancing skeletal muscle mass, enhancing grip strength, enhancing circulating osteocalcin, enlarging the cross-section area (CSA) of the tibialis anterior and/or soleus muscles, increasing skeletal muscle-to-body ratio and/or myofiber CSA, reversing the infiltration of adipose-like tissues by aging, increasing the total protein content in aged skeletal muscle, downregulating the expression level of Fbxo32 or Trim63, reversing aging-related muscle wasting, enhancing force, reversing the aging-related changes in fiber-type switching, reducing the aging-related increase in intramuscular lipid infiltration, collagen deposition or the number of central myonuclei in aged myofibers, enlarging the deep femoral artery or capillary density, reducing body weight progressively, decreasing in whole-body fat mass, increasing lean mass, reversing the aging-related alteration in serum triglyceride or total cholesterol levels, improving glucose tolerance, restoring the expression of Glut4 and Pkm in aged skeletal muscle, enhancing neuromuscular and/or enhancing physical performance in the subject.

In some embodiments, the method as described herein is further for reducing aging-related muscle wasting, frailty score, systemic inflammation, and senescence in the subject.

In some embodiments, the method as described herein is further for restoring aging-related dysregulation.

In some embodiments, the method as described herein is further for increasing muscle mass and physical performance.

In one embodiment, the method as described herein is further for inhibiting multifactorial aging process.

In one embodiment, the subject is an aged subject.

In one embodiment, the subject is a postmenopausal subject or a healthy subject.

In certain embodiments, the composition described herein is a food product comprising the composition.

### BRIEF DESCRIPTION OF DRAWINGS

The patent or application file contains at least one drawing executed in color. Copies of this patent or patent application publication with color drawing(s) will be provided by the Office upon request and payment of the necessary fec.

FIGS. 1A to 1F show that GB enlarges myofiber and grip strength in 6-month-old mice. FIG. 1A: Experimental design and GB structure: C57BL/6 female mice (6-month-old) received GB or vehicle by oral gavage for two months (6m+Vehicle, n=4; 6m+GB, n=6). FIG. 1B: Expression of circulating osteocalcin after GB administration for two months. FIG. IC: Representative H&E cross-section images of tibialis anterior. FIGS. 1D to 1E: Quantification and distribution of tibialis anterior (FIG. 1D) and soleus (FIG. 1E) myofiber cross-sectional area. Results are measured from 11 images of 4 mice for the vehicle group and 15 images of 6 mice for the GB group. FIG. 1F: Forelimb grip strength assay. Quantitative data are presented as mean ±SD in the histogram with data points. Statistical analyses are performed using Student's t-test, with significance set at P<0.05. (*P<0.05; **P<0.01; ***P<0.001).

FIGS. 2A to 2K show that GB ameliorates aging-related skeletal muscle wasting. FIG. 2A: Experimental design: Aged C57BL/6 female mice (20-month-old) received GB or vehicle and young C57BL/6 female mice (3-month-old) received vehicle daily by oral gavage for two months (Young+Vehicle, n=10; Aged+Vehicle, n=14; Aged+GB, n=19). FIG. 2B: Expression of circulating osteocalcin after GB administration for two months. FIG. 2C: Muscle weight-to-body weight. FIG. 2D: Representative H&E cross-section images (upper panel) and longitudinal sections (lower panel) of quadriceps. Scale bar, 100 μm for upper panel; 400 μm for lower panel. The arrow indicates intermuscular fat infiltration. FIGS. 2E to 2F: Quantification (FIG. 2E) and distribution (FIG. 2F) of the myofiber cross-sectional area. FIG. 2G: Quantification of adipose-like tissue area (n=4). (H) Relative protein content of gastrocnemius (n=8). FIGS. 2I to 2J: Detection of Fbxo32 and Trim63 mRNA (FIG. 21) and protein expression (FIG. 2J) in gastrocnemius muscle of aged mice by real-time PCR and western blot. FIG. 2K: Radar chart of 9 muscle wasting phenotypes measured in this study. Tibialis anterior, TA; Extensor digitorum longus, EDL; Gastrocnemius, GA; Solcus, Sol; Quadriceps, QA. Quantitative data are presented as mean ±SD in the histogram with data points. Statistical analyses are performed using one-way ANOVA with Tukey's multiple comparison test. Means not sharing any letter are significantly different (p<0.05).

FIGS. 3A to 3O show that GB administration improves physical activity and ameliorates muscle degeneration in aged mice. FIGS. 3A to 3D: Forelimb grip strength (Young+Vehicle, n=10; Aged+Vehicle, n=14; Aged+GB, n=19) (FIG. 3A), rotarod analysis for exercise capacity (n=10) (FIG. 3B), hanging test for endurance (n=5) (FIG. 3C), and balance beam assay for motor coordination (Young+Vehicle, n=10; Aged+Vehicle, n=13; Aged+GB, n=17) (FIG. 3D) were preformed to access the healthspan in young mice or aged mice with or without GB administration for 2 months. In the balance beam assay, one mouse in the Aged+Vehicle group and two mice in the Aged+GB were unwilling to cross the beam. In the hanging test, all young mice hung over 600 seconds, the fixed hanging limit period. FIGS. 3E to 3H: Ex vivo muscle contractility and fatigability assessment (n=5 for each group). Twitch force (FIG. 3E), tetanic force (FIG. 3F), time to max (FIG. 3G), and the fatigue and postcontraction recovery (FIG. 3H) of GA muscle. FIG. 3I: Representative images of muscle fiber type staining (upper panel), oil red O staining (middle panel), and Masson's trichrome staining (bottom panel) in GA muscle. Scale bar 50 μm. Upper panel: Blue, Type I myofiber; Green, Type IIA myofiber; Red: Type IIB myofiber; Non-stain, Type IIX myofiber. Middle panel: Red, lipid droplet in both bright field and fluorescence images. Bottom panel: Blue, collagen; Red, muscle fibers. FIGS. 3J to 3L: Quantification of muscle fiber type staining (FIG. 3J), oil red O staining (fluorescence images) (FIG. 3K), and Masson's trichrome staining (FIG. 3L). FIG. 3M: Percentage of control nucleus myofibers. Total 10,414, 16,800 and 16,328 myofibers in Young+Vehicle, Aged+Vehicle and Aged+GB groups were counted from HE stained sections, respectively (n=8). FIG. 3N: Quantitative live micro-CT imaging of vascular networks at quadriceps. FA, femoral artery; DFA, deep femoral artery (Aged+Vehicle, n=4; Aged+GB, n=6). FIG. 3O: Representative CD31 staining and quantitation of quadriceps. DAPI is used as a nuclear counterstain. Scale bar, 200 μm for upper images; 100 μm for bottom images. Quantitative data are presented as mean ±SD in the histogram with data points. Statistical analyses are performed using one-way ANOVA with Tukey's multiple comparison test. Means not sharing any letter are significantly different (p<0.05); for panel M, statistical analyses are performed using Student's t-test with significance set at P<0.05. (*P<0.05; **P <0.01; ***P<0.001).

FIGS. 4A to 4J show that GB administration improves metabolic health in aged mice. Aged and young C57BL/6 female mice received GB or vehicle for 2 months (schematic relevant to FIG. 2A). FIG. 4A: Fluctuations of body weight during GB administration (Young+Vehicle, n=10; Aged+Vehicle, n=14; Aged+GB, n=19). Symbols indicate statistically significant differences between the data at the same time point. FIG. 4B: Percent of whole body lean mass and fat mass after GB administration (Young+Vehicle, n=10; Aged+Vehicle, n=14; Aged+GB, n=19). FIG. 4C: Food and water consumption after 2 months of GB administration (n=4). Consumption was normalized to body weight. FIG. 4D: Serum triglyceride and total cholesterol levels (n=5-6). FIG. 4E: Fasting blood glucose after fasting 18 hours. (n=5-6). FIG. 4F: Glucose tolerance test (n=5-6). Symbols indicate statistically significant differences between the data at the same time point. FIGS. 4G to 4H: Expression of glucose metabolism-related genes in skeletal muscle (FIG. 4G) and liver (FIG. 4H) by real-time PCR (n=5-6). FIG. 4I: Insulin sensitivity test (n=5-6) (J) Radar chart of 10 metabolic health phenotypes measured in this study. In the histogram, quantitative data are presented as the means ±SD; in the line chart, quantitative data are presented as the means ±SEM. Dependent on experiment design, statistical analyses are performed using one-way ANOVA with Tukey's multiple comparison test or using Two-way ANOVA with mix-effect model analysis. Means not sharing any letter are significantly different (p<0.05) at indicative time point.

FIGS. 5A to 5D show that GB administration alleviates aging-related frailty. Aged and young C57BL/6 female mice received GB or vehicle for 2 months (schematic relevant to FIG. 2A, n=7 for each group). FIG. 5A: Average frailty score. FIG. 5B: Heatmap analysis of frailty index. Each profile represents an individual mouse. Rows are centered, unit variance scaling is applied to rows. The following rows are constant and were removed: ‘Dermatitis’, ‘Nasal discharge’, ‘Vaginal/uterine/penile prolapse’, ‘Breathing Rate/depth’. FIG. 5C: PCA analysis of frailty index. No scaling is applied to rows; SVD with imputation is used to calculate principal components. X and Y axis show principal component 1 and principal component 2, which explain 55.1% and 9.2% of the total variance, respectively. Prediction ellipses are such that with a probability 0.95, a new observation from the same group will fall inside the eclipse. Since many young mice showed no frailty, some data points in PCA overlap. FIG. 5D: Correlation matrix of the 31 frailty measurements using Pearson's correlation coefficient between the three groups.

FIGS. 6A to 6H show that GB modestly reverses inflammaging and increases muscle beneficial molecules. FIG. 6A: Heatmap of serum cytokine/chemokine profile. Serum was measured by a 40-plex cytokine/chemokine panel (Young+Vehicle, n=4; Aged+Vehicle, n=8; Aged+GB, n=8). Rows are centered; unit variance scaling is applied to rows. Rows are clustered using correlation distance and average linkage. Statistical analyses are performed using by the Kruskal-Wallis procedure. *P<0.05 for Young versus Aged+Vehicle; #p<0.05 for versus Aged+Vehicle versus Aged+GB. FIG. 6B: PCA analysis of 40-plex cytokine/chemokine panel of mouse serum. Each profile represents the serum sample from an individual mouse. Unit variance scaling is applied to rows; singular value decomposition with imputation is used to calculate principal components. X and Y axis show principal component 1 and principal component 2, which explain 43.3% and 12.2% of the total variance, respectively. Prediction ellipses are such that with probability 0.95, a new observation from the same group will fall inside the ellipse. FIG. 6C: Venn diagrams illustrating convergence and non-congruence of top 10 FIT predicted cytokines according to human effect size values from two datasets. FIG. 6D: Expression levels of inflammatory cytokines, IL-6 and INF-γ, in multiple tissues measured by real-time PCR. FIG. 6E: Expression levels of IL-6 in aged MI macrophages (left), MPD C2C12 (middle) and MPD C2C12-derived myotubes (right) after GB treatment for 48 hours. MPD, multiple population doublings. FIG. 6F: Representative IHC images of F4/80 staining in the bone marrow of femur. Scale bar at 500 μm, 200 μm and 50 um from top to bottom panels. FIGS. 6G to 6H: Representative IHC images of CD86 (FIG. 6G) and CD206 (FIG. 6H) staining in the bone marrow of femur. Scale bar at 200 μm, 50 μm and 20 μm from top to bottom panels. Quantitative data are presented as the means +SD in the histogram with data points. Statistical analyses are performed using one-way ANOVA with Tukey's multiple comparison test (Means not sharing any letter are significantly different (p<0.05)) or Student's t-test with significance set at P<0.05. (*P<0.05; **P<0.01; ***P<0.001) depending on experiment design.

FIGS. 7A to 7I show that GB reduced senescence markers in vivo and in vitro. FIG. 7A: Expression levels of senescence markers, p16, p19 and p57, in liver, lung, kidney, spleen, heart and skeletal muscle were measured by real-time PCR (n=6). FIG. 7B: Representative IHC images of γ-H2AX (brown) in multiple tissues. Scare bar at 40 μm. FIG. 7C: Experimental design and the senescence-related assessments. Senescence was induced by H₂O₂ stimulation for 2 hrs in human MSCs; after PBS wash, the senescent MSCs were treated with or without GB (5 mg/L). Sen, H202-induced senescence. FIG. 7D: Cellular morphology and cell size of senescent human MSCs with or without GB treatment (n=25-32 cells). Scale bar at 100 μm. FIG. 7E: Relative cell number of senescent human (n=8). Symbols indicate statistically significant differences between the data at the same time point. FIG. 7F: Cellular viability was measured by CCK8 assay (n=8). FIG. 7G: Expression levels of senescence markers (n=3). FIG. 7H: Representative images and proportion of SA-β-gal positive cells (n=3). FIG. 7I: Representative images of DAPI staining for Senescence-associated heterochromatin foci.

FIGS. 8A to 8H show single-nucleus landscape of skeletal muscles. (A) Graphical scheme of the experimental design. Nucleus of GA muscle was stained with DAPI and isolated by flow cytometry method. FIGS. 8B to 8C: Flow cytometry gating strategy (FIG. 8B) and isolated nucleus with DAPI staining (FIG. 8C). Scale bar at 20 μm and 5 μm from top to bottom panels. FIGS. 8D to 8E: UMAP diagram from integrated datasets (FIG. 8D) and separated datasets (FIG. 8E) revealed the coordinates of nuclear types in GA muscle of Young+Vehicle, Aged+Vehicle and Aged+GB groups. Type 2B myonucleus-1, Type 2B-1; Type 2B myonucleus-2, Type 2B-2; Type 2X myonucleus, Type 2X; Type 2A myonucleus, Type 2A; myotendinous Junction, MTJ; fibro/adipogenic progenitors, FAPs; endothelial cells, EC; pericytes, Peri; satellite cells, SatC; adipocytes, Adip; tenocytes, Teno; immune cells, IC; smooth muscles SM; Schwann cells, SchwC. FIG. 8F: Venn diagrams illustrating the reversion of nuclear types by GB administration. Percentage change in proportion over 10% was considered significant. FIG. 8G: Rose diagrams revealed the numbers of aging DEGs, GB DEGs, and Rescue DEGs of various cell types in skeletal muscles. FIG. 8H: Rescue DEGs from various cell types and representative GO pathways in skeletal muscles.

FIGS. 9A to 9M show that single-nucleus RNA sequencing analysis revealing aging-related changes in myonuclei is reversed by GB administration. FIGS. 9A to 9B: UMAP diagrams from integrated datasets (FIG. 9A) and separated datasets (FIG. 9B) revealed the myonucleus pattern in Young+Vehicle, Aged+Vehicle and Aged+GB groups. Red arrow indicates the Runx1+type 2b myonuclei. FIG. 9C: Heatmap of representative differentially expressed genes demonstrates the correctness of clustering in the integrated dataset. FIG. 9D: UMAP diagram and violin plots of integrated datasets revealing the expression level of conventional myo-lineage genes for myonucleus type annotation. FIG. 9E: Distribution of Myh1, Myh3, Myh4, and Myh7 in myonuclei in the integrated dataset. FIG. 9F: Visualized composition of nuclear types. FIG. 9G: Venn diagrams illustrating the reversion of myonucleus types by GB administration. FIG. 9H: Rose diagrams revealed the numbers of aging DEGs, GB DEGs, and Rescue DEGs in myonucleus subclusters. FIG. 9I: Dot plot of Rescue DEGs in myonucleus subclusters. FIG. 9J: GO enrichment analysis by biological process of Rescue DEGs in myonuclei. FIG. 9K: Distribution and expression level of Runx1 in myonucleus. FIG. 9L: GO enrichment analysis by biological process (upper panel) and KEGG pathways analysis (bottom panel) of aged Runx1+type2B myonuclei. FIG. 9M: Top 20 enriched Gene Ontology terms of each myonuclei cluster found from cluster-specific DEGs. Yellow box highlights the unique pathways of Runx1+type2B myonuclei.

FIGS. 10A to 10E show that single-nucleus RNA sequencing analysis reveals the changes in cell-cell communication. FIGS. 10A to 10B: Circle plots (FIG. 10A) and heatmap (FIG. 10B) showed the differential cell-cell communication between Young+Vehicle vs Aged+Vehicle and Aged+GB vs Aged+Vehicle datasets in skeletal muscle. The similar signaling pattern between the two datasets indicates GB reversed aging-related changes in cell-cell communication. FIG. 10C: Plot revealed the contribution of outgoing and incoming signaling between various cells. Circle size represents the counts in each nucleus types. FIG. 10D: Chord diagram shows interaction count and weight of cell-to-muscle communication. The thickness of the line represents interaction count and weight of ligand-receptor interaction. FIG. 10E: Differential cell-to-muscle signaling.

FIGS. 11A to 11F show that late-life GB administration extends lifespan in aged mice. FIG. 11A: A Chronic GB administration beginning from 20 months of age (n=35 per group), and the morphology of mice around 30 months of age with or without GB administration. FIGS. 11B to 11D) Survival curve, (FIG. 11B) maximum lifespan of 10% and 20% longest-living (FIG. 11C) and age-associated mortality rates (FIG. 11D) of vehicle-and GB-treated aged mice. FIG. 11E: Cumulative tumor incidence curve. Tumor incidence was recorded at necropsy in vehicle-and GB-treated aged mice. FIG. 11F: Survival curve separated by tumor incidence. The survival curve and cumulative tumor incidence curve were plotted using the Kaplan-Meier method. The median lifespan (panel B and F) and the day of 50% cumulative cancer incidence (panel E) are indicated of the figures. Statistical analyses were performed using the log-rank test and Gehan-Breslow-Wilcoxon test and presented in blue and orange, respectively.

### DETAILED DESCRIPTION OF THE INVENTION

Unless defined otherwise, all scientific or technical terms used herein have the same meaning as those understood by persons of ordinary skill in the art to which the present invention belongs. Any method and material similar or equivalent to those described herein can be understood and used by those of ordinary skill in the art to practice the present invention.

It must be noted that, as used in the specification and the appended claims, the singular forms “a,” “an” and “the” include plural referents unless the context clearly dictates otherwise. Thus, unless otherwise required by context, singular terms shall include the plural, and plural terms shall include the singular.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein, the term “about” refers to ±20%, preferably ±10%, and even more preferably ±5%.

The term “and/or” is used to refer to both things or either one of the two mentioned.

The term “preventing” or “prevention” is recognized in the art, and when used in relation to a condition, it includes administering, prior to onset of the condition, an agent to reduce the frequency or severity of or to delay the onset of symptoms of a medical condition in a subject, relative to a subject which does not receive the agent.

The terms “treatment,” “treating,” and “treat” generally refer to obtaining a desired pharmacological and/or physiological effect. The effect may be preventive in terms of completely or partially preventing a disease, disorder, or symptom thereof, and may be therapeutic in terms of a partial or complete cure for a disease, disorder, and/or symptoms attributed thereto. “Treatment” as used herein covers any treatment of a disease in a mammal, preferably a human, and includes (1) suppressing development of a disease, disorder, or symptom thereof in a subject or (2) relieving or ameliorating the disease, disorder, or symptom thereof in a subject.

As used herein, the term “disorder” is used interchangeably with “disease” or “condition.”

As used herein, the term “subject” is any animal that can benefit from the administration of a compound or composition as disclosed herein. In some embodiments, the subject is a mammal, for example, a human, a primate, a dog, a cat, a horse, a cow, a pig, a rodent, such as, for example, a rat or mouse. Typically, the mammal is a human.

The term “effective amount” of an active ingredient as provided herein means a sufficient amount of the ingredient to provide the desired regulation of a desired function. As will be pointed out below, the exact amount required will vary from subject to subject, depending on the disease state, physical conditions, age, sex, species and weight of the subject, the specific identity and formulation of the composition, etc. Dosage regimens may be adjusted to induce the optimum therapeutic response. For example, several divided doses may be administered daily or the dose may be proportionally reduced as indicated by the exigencies of the therapeutic situation. Thus, it is not possible to specify an exact “effective amount.” However, an appropriate effective amount can be determined by one of ordinary skill in the art using only routine experimentation.

Ginkgolide B (GB) is a small molecule from Ginkgo biloba, having the following structure:

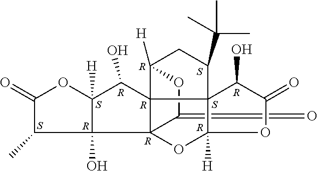

Whether GB improves overall healthspan and longevity remains unknown. The present disclosure surprisingly found that GB can improve overall healthspan and longevity and thus provides methods for improving healthspan and longevity by using GB or a derivative thereof.

In the present disclosure, the following embodiments are provided. Lifelong oral GB administration in mice starting from 20 months' old extended lifespan by 20% alongside reduced tumor incidence. GB administration for 2 months significantly improved healthspan as evidenced by comprehensive assessments in the context of muscle quality, physical activity, metabolism, frailty index, inflammation, and senescence. GB-administrated aged mice displayed enhancements in skeletal muscle mass, grip strength, glucose tolerance, neuromuscular and physical performance, and reduction in frailty score, systemic inflammation, and senescence, which were towards the levels of young mice. GB administration also ameliorated several aging-related pathologies without apparent adverse effects. By single-nucleus RNA sequencing (snRNA-seq), we revealed that GB partially restored the aging-related dysregulation in cell-type composition, intracellular signaling pathways, and cell-cell communication in skeletal muscle. Notably, GB reduced the quantity of aging-induced novel Runx1+type 2B myonuclei, which are particularly associated with an apoptotic burden and aging-related signatures. Additionally, GB administration increased muscle mass and physical performance in both postmenopausal and healthy adult (6-month-old) mice. In summary, we uncovered a novel function of GB in extending healthspan in pathological and steady-state conditions to extend lifespan. The rejuvenating effects of GB against the multifactorial aging process provide promising prospects for achieving healthy aging in humans.

In some embodiments of the disclosure, GB has been evaluated across more than 100 parameters related to aging and healthspan in the naturally aging animal models, the gold standard animal for aging study. GB reverses aging by targeting multiple hallmarks of aging, including genomic instability, loss of proteostasis, deregulated nutrient sensing, cellular senescence, stem cell exhaustion, and altered intercellular communication, as well as by targeting various aging-related syndrome, such as osteoporosis, sarcopenia, inflammaging, frailty, spontaneous tumor.

One significant aspect of GB's effectiveness lies in its senotherapeutic properties, which are most important for enhancing healthspan and extending lifespan. Senescence, the state in which cells lose their function and ability to divide properly, not only contributes to aging but also plays a causal role in numerous age-related diseases. GB exhibits senomorphic effects, reversing the senescence phenotype in multiple tissues and stem cells. Indeed, GB reverses senescence in MSCs, which play a crucial role in tissue regeneration and repair. This indicates the ability of GB to rejuvenate essential cellular functions and promote tissue health, further supporting its potential as an anti-aging intervention.

In some embodiments of the disclosure, the multifaceted action of GB, targeting multiple interconnected aging hallmarks simultaneously, sets it apart as a promising candidate for reversing aging. By addressing these fundamental processes, GB holds the potential to restore cellular and tissue function, enhance overall healthspan, and potentially extend lifespan. Continued research and exploration are necessary to fully understand and maximize the potential of GB in reversing the aging process and promoting healthy aging.

In some embodiments of the disclosure, GB has been shown to exhibit significant antioxidant activity by scavenging ROS and reducing oxidative stress. One of the mechanisms by which GB exerts its anti-ROS effects is through its ability to inhibit the production of ROS. It can suppress the activity of enzymes involved in ROS generation, such as NADPH oxidase and xanthine oxidase, thereby reducing the overall levels of ROS in cells. Additionally, GB can enhance the activity of endogenous antioxidant defense systems. It can upregulate the expression and activity of antioxidant enzymes, including superoxide dismutase (SOD), catalase, and glutathione peroxidase. These enzymes help to neutralize and detoxify ROS, thus preventing oxidative damage. Furthermore, ginkgolide B has been shown to possess direct free radical scavenging activity. It can directly interact with ROS and neutralize their harmful effects, thereby protecting cells from oxidative stress-induced damage.

Administering compounds and/or pharmaceutical compositions to a subject may involve administering therapeutically effective amounts, which means an amount of compound effective in treating the stated conditions and/or disorders in a subject. Such amounts generally vary according to a number of factors well within the purview of ordinarily skilled artisans. These include, without limitation: the particular subject, as well as its age, weight, height, general physical condition, and medical history, the particular compound used, as well as the carrier in which it is formulated and the route of administration selected for it; and, the nature and severity of the condition being treated.

Administering typically involves administering pharmaceutically acceptable dosage forms, which means dosage forms of compounds described herein, and includes, for example, tablets, dragees, powders, elixirs, syrups, liquid preparations, including suspensions, sprays, inhalants tablets, lozenges, emulsions, solutions, granules, capsules, and suppositories, as well as liquid preparations for injections, including liposome preparations. Techniques and formulations generally may be found in Remington's Pharmaceutical Sciences, Mack Publishing Co., Easton, Pa., latest edition, which is hereby incorporated by reference in its entirety. Administering may be carried out orally, topically, transdermally, parenterally, subcutaneously, intravenously, intramuscularly, intraperitoneally, by intranasal instillation, by intracavitary or intravesical instillation, intraocularly, intraarterially, intralesionally, or by application to mucous membranes. Compounds may be administered alone or with suitable pharmaceutical carriers, and can be in solid or liquid form, such as tablets, capsules, powders, solutions, suspensions, or emulsions.

Compounds or compositions for oral administration may be in the form of tablets, troches, lozenges, aqueous or oily suspensions, granules or powders, emulsions, capsules, syrups or elixirs. Orally administered compounds or compositions may contain one or more agents, such as, sweetening agents such as sucrose and lactose, flavoring agents such as peppermint, oil of wintergreen or cherry, coloring agents and preserving agents to provide a pharmaceutically palatable preparation. Moreover, compounds or compositions in tablet form may be coated to delay disintegration and absorption in the gastrointestinal tract thereby providing a sustained action over an extended period of time. A time delay material such as glycerol monostearate or glycerol stearate may be used.

Aqueous suspensions containing a compound of the present disclosure may also contain one or more preservatives, such as, for example, ethyl or n-propyl-p-hydroxy-benzoate, one or more coloring agents, flavoring agents or sweetening agents.

The composition can be, for example, a capsule, tablet, drink, powder or dairy product. Preferably, the present composition is a nutraceutical or a pharmaceutical product, a nutritional supplement, or medical food.

Examples of the compositions of the disclosure are nutritional compositions, including food products, and in particular, dairy products.

Nutritional compositions of the disclosure also include food supplements, and functional food. A “food supplement” designates a product made from compounds usually used in foodstuffs, but which is in the form of tablets, powder, capsules, potion or any other form not usually associated with aliments, and which has beneficial effects on one's health. A “functional food” is an aliment which also has beneficial effects on one's health. In particular, food supplements and functional food can have a physiological effect -- protective or curative --against a disease.

If the composition according to the disclosure is a dietary supplement, it can be administered as such, can be mixed with a suitable drinkable liquid, such as water, yoghurt, milk or fruit juice, or can be mixed with solid or liquid food. In this context, the dietary supplement can be in the form of tablets, pills, capsules, lozenges, granules, powders, suspensions, sachets, pastilles, sweets, bars, syrups and corresponding administration forms, usually in the form of a unit dose. Preferably, the dietary supplement comprising the composition of the disclosure is administered in the form of tablets, lozenges, capsules or powders, manufactured in conventional processes of preparing dietary supplements.

The dosage of the present compounds or compositions depends on the route and frequency of administration, as well as the age, weight and physical condition of the patient. The appropriate dosage of the compounds or compositions can be readily determined by the skilled medical practitioner.

### EXAMPLES

### Materials and Methods

### Animal Models and Care

All animals were purchased from Laboratory Animal Services Centre, the Chinese University of Hong Kong, ad libitum fed the standard diet (#5V0f, LabDiet, St. Louis, MO, USA), and housed at specific-pathogen-free and humidity-controlled conditions under a 12 light: 12 dark photoperiod with temperature at 20-22° C. Animals were kept up to five per cage and were randomly assigned to control and experimental groups in all experiments. We assured that all animals received humane care according to the criteria outlined in the “Guide for the Care and Use of Laboratory Animals” prepared by the National Academy of Sciences and published by the National Institutes of Health (NIH publication 86-23, revised 1985). For the short-term experiment, 3-(young), 6-(adult) and 20-month-old (aged) female C57BL/6 mice were administered 12 mg/kg body weight GB (#G6910, Sigma-Aldrich, St. Louis, MO, USA) or vehicle daily by oral gavage for two months. For the postmenopausal mouse model, female C57BL/6 mice were ovariectomized (OVX) bilaterally at 6 weeks of age. Post-surgery for three weeks, the mice received GB (3 and 12 mg/kg body weight) daily for two months. GB was diluted to the appropriate concentration in 100 μL of sterile PBS (#10378016, Gibco, Waltham, MA, USA). For lifespan and cancer incidence analysis, 20-month-old female C57BL/6 mice received 12 mg/kg body weight GB daily without being used for experiments until natural death or sacrifice according to the recommendations from veterinary technician due to severe discomfort. The euthanized animals were considered to have lived to the end of natural lifespans. Survival curves were illustrated with Kaplan-Meier methods with log-rank test by using GraphPad Prism 8 (GraphPad Software, Inc.). Maximum lifespans refer to the mean of the 80th and 90th percentile of the distribution of lifespan, which was obtained by comparison between of control and GB groups for using Student's t-test. Age-associated mortality rate (qx) was calculated by cumulative dead number of animals at the end of interval (100 days) over the total number of animals. The hazard rate (hz) was calculated by hz=2 qx/(2-qx), and natural logarithm of hz was plotted against time. The GB concentration 3 and 12 mg/kg body weight we used in mice were equivalent to the human doses of 0.3 and 1.2 mg/kg body weight. Animal experiments were approved by Animal Experimentation Ethics Committee of the Chinese University of Hong Kong.

### Food and Water Intake Measurement

For the food and water intake measurement, mice were divided into individual animal cages individually for 3 days for stabilizing the animals, and then daily consumption was recorded. The values were calculated by adjusting for body weight.

### Body Composition and Vascular Networks Quantification

Body composition was measured by Minispec LF50 TD-NMR (Bruker, MA, USA) based on nuclear magnetic resonance methods. Briefly, mice were anesthetized for the duration of the procedure by 3% isoflurane (Zoctis, London, UK) and placed in the scanning chamber in the prone position, with the limbs and tail stretched away from the body. The analysis would be conducted by default software per manufacturer's protocol. The vascular networks at vascular networks were quantified by in vivo micro-computed tomography (U-CT, Milabs, Netherlands). Briefly, mice intravenously received 150 μL ExiTron™ nano 12000 (#130-095-698, Miltonic Biotec GmbH, Bergisch Gladbach, Germany), and then mice were anesthetized with isoflurane before being positioned on the scanning chamber. After scanning, the blood vessels were analyzed by a curved multiplanar reconstruction algorithm by using Osirix software (Bernex, Switzerland).

### Frailty Index

The noninvasive 31-item frailty index established by Whitehead et al. was used to evaluate frailty in mice (Whitehead, J. C. et al. A clinical frailty index in aging mice: comparisons with frailty index data in humans. J Gerontol A Biol Sci Med Sci 69, 621-632, doi:10.1093/gerona/glt136 (2014)). Whole assessments were conducted by blind investigators as possible. The scored items represent the aging-related deteriorations based on the criteria in clinical practice, consisting of the integument, musculoskeletal system, vestibulocochlear and auditory systems, ocular and nasal systems, digestive system, urogenital system, respiratory system, signs of discomfort, body weight and body surface temperature. Surface body temperature was determined by an infrared thermometer (Thermo Distance, Terraillon, Croissy-sur-seine, France), and grip strength was measured by the standard equipment described in the later section. The other subjective observations were scored by researchers. The scores 0, 0.5 and 1 indicated no sign of frailty, moderate defect and severe defect, respectively. For the body temperature, grip strength and body weight scoring, the average and standard deviation (STDEV) values were used as a baseline. A change within one STDEV is scored as a 0, a decreased change over one STDEV but lower than two STDEV was scored as a 0.5 and a change more than two STDEV was scored as a 1. The heatmap was conducted by an online tool, Clust Vis. The principal component analysis (PCA) and correction analysis were conducted by MetaboAnalyst (Version 5).

### Physical Functional Analysis

Forelimb grip strength was measured by a grip strength meter (Muromachi Kikai Co., Tokyo, Japan). Each mouse performed five trials for force measurement, and the values were normalized to body and recorded. A rotarod test was carried out by using the rotarod treadmill (Singa Technology Corporation, Taoyuan City, Taiwan). The speed was adjusted to 4 rpm for 4 sec, 10 rpm for 10 sec, 20 rpm for 20 sec, and then 40 rpm until the mice fell. Mice were familiarized with the rotarod and balance beam by running 5 min for three consecutive days before the onset of experimentation. During the experiment, each mouse performed four trials, and the average time that each rodent was able to stay on top of the rotarod treadmill was recorded. The balance beam test was used to assess subtle motor coordination and balance. After initial training, mice were given four consecutive trials where they were required to cross 60 cm of a round beam (14 mm in diameter). The time for crossing the beam and the number of hind-feet slip were recorded. Hanging endurance was determined by Kondziella's inverted hanging screen test. Mice were putted at the center of grasped wire screen (bar thickness, 2 mm; mesh, 10 mm). After inverting the wire screen, the time of sustained limb tension on the screen were recorded. A fixed maximum hanging limit of 600 seconds was used. Each trial was repeated three times at 30-min interval, the mean values were recorded.

### Intraperitoneal Glucose Tolerance Test (IPGTT) and Insulin Tolerance Test (IPITT)

Mice fasted 16 hours and 2 hours before performing an IPGT test and IPIT test, respectively. IPGTT was performed by intraperitoneal injection of fasted mice with 1% body weight of sterile glucose/PBS (20% weight/volume). IPITT was performed by intraperitoneal injection of fasted mice with insulin (0.75 unit/kg body weight) (#12585014, Gibco). Blood glucose levels were measured at indicative timepoints after injection by a blood glucometer (countour plus, Ascensia Diabetes Care, Hong Kong).

### Ex Vivo Skeletal Muscle Functionality Measurement

For the ex vivo muscle function test, the isolated right side gastrocnemius (GA) muscle were incubated in Krebs buffer at room temperature and with 95% O2 and 5% CO2 for five minutes. GA muscle received stimuli three times under the optimal length to evaluate twitch force. Three 150 Hz continuous stimuli were given to evaluate tetanic force. Twitch force and tetanic force were normalized to the cross-section area (CSA) of GA muscle. GA muscle fatigue was measured by repeated isometric tetanic contraction every 5 seconds for a total of 60 contractions. Five minutes and ten minutes after fatigue protocol, the maximal tetanic force was measured to assess post-fatigue recovery. All of the results were recorded with the Dynamic Muscle Control system (DMC v5.4; Aurora Scientific, Inc.), and results were analyzed by the Dynamic Muscle Analysis system (DMA v3.2; Aurora Scientific, Inc.).

### Histochemical Staining

Tissue samples were harvested and embedded in paraffin or in OCT (#4583, Sakura Finetek USA, Torrance, CA, USA). For paraffin-embedded samples, 10 μm sections were deparaffinized by xylene and alcohol gradient treatment and were then hydrated by distilled water and processed for heat-induced antigen retrieval with antigen retrieval solution at 70° C. for 1.5 hours before staining. For OCT-embedded samples, 10 μm sections were hydrated by distilled water before staining. Hematoxylin and cosin staining (#MHS16 and #HT110116 Sigma-Aldrich), oil red O staining (#00625, Sigma-Aldrich), and Masson's trichrome staining (#HT15, Sigma-Aldrich) were used to assess the histology, lipid droplet, and collagen deposition. For immunohistochemical staining, antigen-retrieval samples were blocked with 5% bovine serum albumin (BSA) (#A9647, Sigma-Aldrich) for 1 hour at room temperature and incubated with primary antibodies overnight at 4° C., followed by a ready-to-use IHC/ICC Kit (#K405, BioVision, Milpitas, CA, USA) at room temperature. The samples were then suspended with double-distilled water and then stained with hematoxylin. For immunocytochemical staining, samples were blocked with 5% BSA (Sigma-Aldrich) for 1 hour at room temperature and incubated with primary antibodies overnight at 4° C., then washed with PBS for ten minutes, and then incubated with secondary antibody for 1 hour at room temperature, then washed with PBS three times for ten minutes. After DAPI staining (#ab228549, Abcam, Cambridge, UK), the samples were mounted for analysis.

### Western Blot

RIPA buffer (#ab156034, Abcam) containing 1X protease/phosphatase inhibitor (#1861281, Thermo Fisher Scientific) was used to lyse samples. Protein content was measured by BCA Protein Assay Kit (#23225, Thermo Fisher Scientific). Samples were loaded and separated in polyacrylamide gels and then transferred to PVDF membranes (#IPVH00010, Merck Millipore, Massachusetts, USA). The membranes were incubated with 5% BSA at room temperature for one hour and then incubated with primary antibodies overnight at 4° C., and then incubated with HRP-conjugated secondary antibodies (#SA00001-1, #SA100002-1, Proteintech, Rosemont, IL, USA) for one hour at room temperature. Signals were detected by the chemiluminescent substrate (#34095, Thermo Fisher Scientific) and were acquired by the ChemiDoc MP imaging system (Bio-Rad, Hercules, CA, USA).

### Circulating Inflammation Cytokines, Chemistry and Osteocalcin Analysis

Serum was collected by cardiac puncture upon sacrifice. For inflammatory cytokine analysis, serum samples were analyzed on a Mouse Inflammation Array G1 (#AAM-INF-G1-4, RayBiotech, Norcross, GA, USA) according to the manufacturer's protocol. Fluorescence intensity was quantified and normalized using the RayBiotech Analysis Tool. The heatmap and PCA were conducted by an online tool, Clust Vis (Metsalu, T. & Vilo, J. ClustVis: a web tool for visualizing clustering of multivariate data using Principal Component Analysis and heatmap. Nucleic Acids Res 43, W566-570, doi: 10.1093/nar/gkv468 (2015)). The Found In Translation methodology (FIT, package version 1.2) was used to extrapolate the mouse serum profile to equivalent human condition. For blood chemistry, samples were loaded on SPOTCHEM test strips and measured by a SPOTCHEM™ dry-chemistry clinical analyzer (ARKRAY Inc. Kyoto, Japan). The expression levels of circulating osteocalcin (#NBP2-68151, Novus Biologicals, Centennial, Co, USA) were quantified by ELISA and were analyzed by a microplate reader (Infinite 200, TECAN, Männedorf, Switzerland).

### In Vitro Cell Culture

C2C12 myoblast cell line (#CRL-1772TM, ATCC, Manassas, VA, USA) was expanded in high-glucose DMEM (#D7777, Sigma-Aldrich) with 10% FBS (#10270106, Gibco, Waltham, MA, USA) and 1% PSG (#10378016, Gibco). Passage 15-20 C2C12 was considered as multiple population doublings C2C12. Subconfluent C2C12 was differentiated to myotubes by myogenic induction medium, high-glucose DMEM (Sigma-Aldrich) containing 2% horse scrum (#16050122, Gibco) for 5 days. Aged bone-marrow monocytes were isolated from 20-month-old female C57BL/6 mice and then differentiated into macrophages in RPMI 1640 (#11875085, Gibco) supplemented with 10% FBS and 20 ng/ml M-CSF (#315-02, PeproTech, Cranbury, NJ, USA). Inflammatory MI macrophages were induced by 45 ng/mL IFN-γ (#485-MI-100, R&D Systems, Minneapolis, MN, USA) +100 ng/mL LPS (#L4391, Sigma-Aldrich). The indicative cells were then treated with or without GB (5 mg/L) for 48 hr. Commercial human mesenchymal stem cells (MSCs)40 were cultured in MSC maintenance medium consisting of IMDM, 10% FBS (#10270106, Gibco), 10 ng/ml bFGF (#233-FB, R&D Systems) and 1% PSG (#10378016, Gibco). To induce senescence, we treated cells at approximately 50% confluence with 200 μM H₂O₂ for 2 hr and then washed cells by PBS two times to remove H202. The cells were treated with GB (5 mg/L) or vehicle in fresh growth medium and subjected to the subsequent experiments at indicative time point.

### Assessment of Senescent Phenotype In Vitro

Cell viability was measured by a CCK-8 kit (#ab228554, Abcam), and cell proliferation was determined by manually cell counting with hemocytometer. Senescence-associated β-galactosidase activity was determined by senescence-associated β-galactosidase staining kit (#9860, Cell Signaling Technology, Danvers, Massachusetts, USA) according to the manufacturer's instructions. Senescence-associated heterochromatin foci were visualized by DAPI staining.

### Single Nuclei Isolation and Single-Nucleus RNA Sequencing

Single-nucleus isolation was performed as previously described 41. GA muscles were isolated from mice immediately following euthanasia. The muscles were minced and homogenized with homogenization buffer, 0.25M sucrose and 1% BSA diluted in Mg²⁺-free, Ca²⁺-free, RNase-free PBS (#AM9624, Invitrogen, Grand Island, NY, USA). The homogenates were incubated with 2.5% Triton-X100/RNase-free PBS at a 1:6 ratio for five minutes. After being filtered through a 100-um strainer, samples were centrifuged at 3000×g for 10 min at 4° C., resuspended in 2% BSA/RNase-free PBS, and then filtered through a 40-μm strainer again. Nuclei were labeled by DAPI with 0.2 U/μL RNase inhibitor (#N8080119, Thermo Fisher Scientific). Labeled nuclei were sorted by FACS Aria II Cell Sorter with 70 μm nozzle (BD Biosciences, San Jose, CA, USA) into 2% BSA/RNase-free PBS with RNase inhibitor. Isolated nuclei were observed by confocal fluorescent microscope (Leica TCS SP8, Wetzlar, Germany) to confirm integrity. To adjust the optimal concentration of nuclei for the 10X Chromium system, isolated nuclei were counted and diluted or concentrated by centrifugation at 250×g for 5 min at 4° C. Single-nucleus RNA-sequencing libraries were constructed by the Chromium Single Cell 3′ reagent kit v2 (10X Genomics, Pleasanton, CA, USA) per the manufacturer's protocol. Following the library preparation, the libraries were sequenced on the NovaSeq 6000 System (Illumina, San Diego, CA, USA) and the 10x Genomics cellranger count pipeline produced 6072 single-nucleus transcriptomes (2128, 2015 and 1929 transcriptomes for Young+Vehicle, Aged+Vehicle, and Aged+GB groups) with 25453, 27,741, and 34,120 reads per nuclei, and 1018, 923, and 1061 genes per nuclei for Young+Vehicle, Aged+Vehicle, and Aged+GB groups respectively.

### Single-Nucleus Transcriptome Analysis

The single-cell feature-barcode matrices from 10x Genomics cellranger count pipeline were imported into R version 4.1.3 and processed with Seurat 4.2.0. Next, ambient RNA was corrected by SoupX package43. Cells with less than 250 features or more than 5% mitochondrial gene expression were first filtered out by using Seurat's suggested protocol. Cell cycle genes were corrected for as described in Seurat's Cell-Cycle Scoring and Regression vignette. Counts were then normalized and scaled using Seurat's SCTransform protocol as suggested by Seurat's vignette. All remaining single-nuclei of each sample were then processed with dimension reduction and Louvain clustering as per Seurat's standard vignette. A combined Seurat object was also created following Seurat's integration protocol as described in Seurat's scRNA-seq integration using default parameters. Dimension reduction and clustering were also performed on the combined dataset as described above. Clusters were annotated using established cell type markers. Cell-types from the combined dataset that expressed high Ttn levels (Type 2B-1, Type 2B-2, Type 2X, Type 2A, MTJ) for each sample were further selected out and recombined into a myonucleus-only dataset using Seurat's integration protocol described above. Dimension reduction and clustering were also performed on the myonucleus dataset as described above, and myonuclei subtypes were annotated using established cell markers. Pseudo-bulk differentially expressed genes (DEGs) were identified using FindMarkers function from Seurat. DEGs for each cluster were identified using Seurat's FindAllMarkers function. Pathway enrichment analysis and gene-set enrichment analysis (GSEA) were performed using clusterProfiler v4.4.4. DEGs with FDR<0.05 were considered for GO enrichment analysis. For GSEA, all genes were considered and ranked by (sign(fold-change)*-log10(adj. p-value)). CellChat v1.5.0 was used to identify intercellular communication patterns between all cell types and myonuclei. CellChat analysis was done on the combined dataset and myonuclei only dataset following the standard and default set-up.

### RNA Extraction and Quantitative PCR

RNAs were purified by using the TRIzol® (#15596018, Invitrogen) and chloroform method (#C7559, Sigma-Aldrich) and reversed transcribed using High-Capacity cDNA Reverse Transcription Kit (#4368814, Applied Biosystems, Foster City, CA, USA). The quantitative RT-PCR was conducted with universal probe system (Roche Life Science, Indianapolis, IN, USA) by using a QuantStudio™ 7 Flex Real-Time PCR System (Applied Biosystems) and was normalized by the expression of GAPDH.

### Radar Charts

Values for radar charts were normalized to young mice (no difference=100%). Data are presented as the average value of individual measurements.

### Image Acquisition and Analysis

For H&E stained sections, images were acquired by Pannoramic Scanner (3DHISTECH Ltd, Budapest, Hungary). For immunohistochemical and immunocytochemical sections, images were acquired by IX83 Inverted Fluorescence Microscope (Olympus Corporation, Tokyo, Japan) or Nikon Ti-2 Inverted Fluorescence Microscope (Nikon Corporation, Tokyo, Japan). Senescence-associated heterochromatin foci staining was acquired by confocal microscope (ZEISS LSM 900; ZEISS, Oberkochen, Germany). Quantification of the results in images was assessed using the same parameters by FUJI software or by Case Viewer (3DHISTECH Ltd).

### Statistical Analysis

Quantitative data are presented as mean +SD in histograms with data points or as mean ±SEM in line graphs. Statistical analyses were performed using one-way ANOVA Tukey's post hoc comparison or unpaired Student's t-test depending on the experimental design by GraphPad Prism 9 (GraphPad Software, Inc., San Diego, CA, USA). The sample sizes and corresponding analysis methods are listed in the figure legends. Statistical values of P<0.05 were considered statistically significant.

### Example 1: GB Increases Muscle Mass and Grip Strength Under Steady-State Conditions

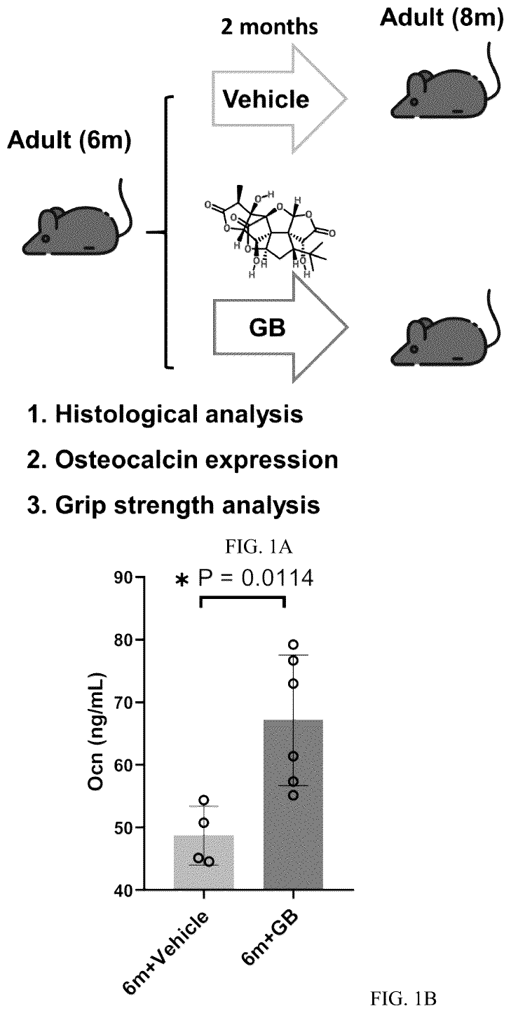

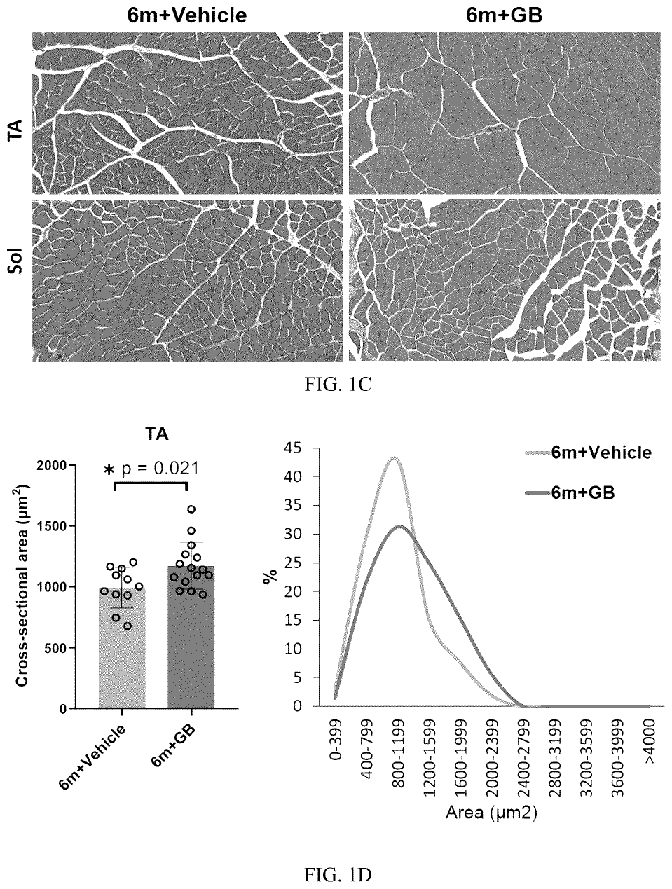

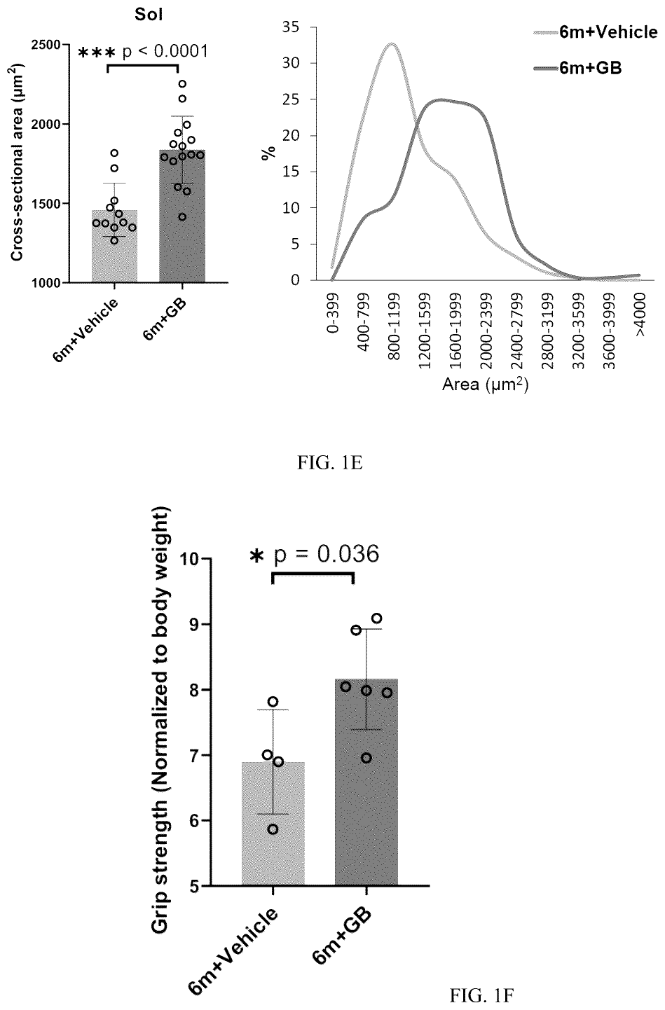

Our initial validation was focused on sarcopenia, pathological declines in muscle mass and strength with age, which substantially devastates quality of life and significantly impacts healthspan and lifespan in the elderly. The maintenance of muscle mass and strength in adulthood reduces the risk of sarcopenia and improves healthspan. Osteocalcin is necessary to maintain muscle mass and health throughout the whole life, and loss of osteocalcin after the age of 30 leads to muscle loss. Since GB increased osteocalcin in aged osteoblasts, we postulated that oral GB administration could increase osteocalcin and skeletal muscle mass in mice. In a proof-of-principle experiment, we gave GB to 6-month-old female mice equivalent to 30-year-old women for two months (FIG. 1A). After treatment, GB-administrated mice exhibited a significantly higher circulating osteocalcin, indicating the bioavailability of GB in vivo (FIG. 1B, p=0.0114). GB administration enlarged the CSA of the tibialis anterior and soleus muscles (FIGS. 1C to 1E, p=0.021 and <0.0001, respectively) and increased forelimb grip strength (FIG. IF, p=0.036). GB administration served as a preventative measure for sarcopenia, suggesting it as a potential treatment for improving healthspan and lifespan.

### Example 2 GB Alleviates Aging-Related Muscle Wasting

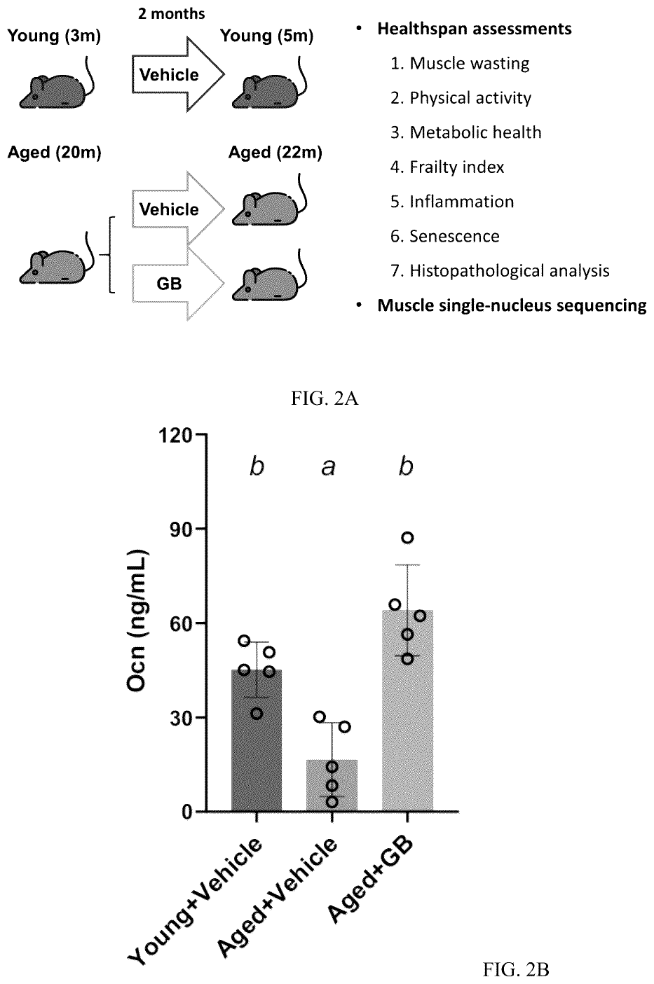

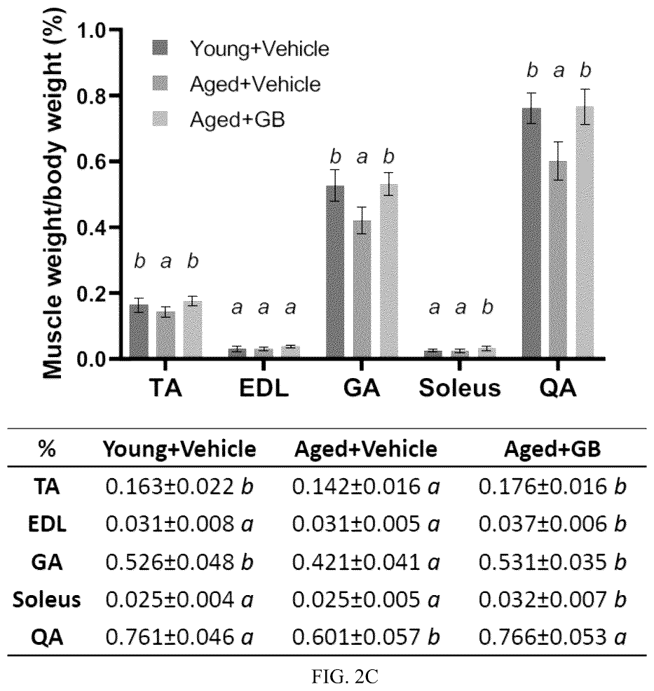

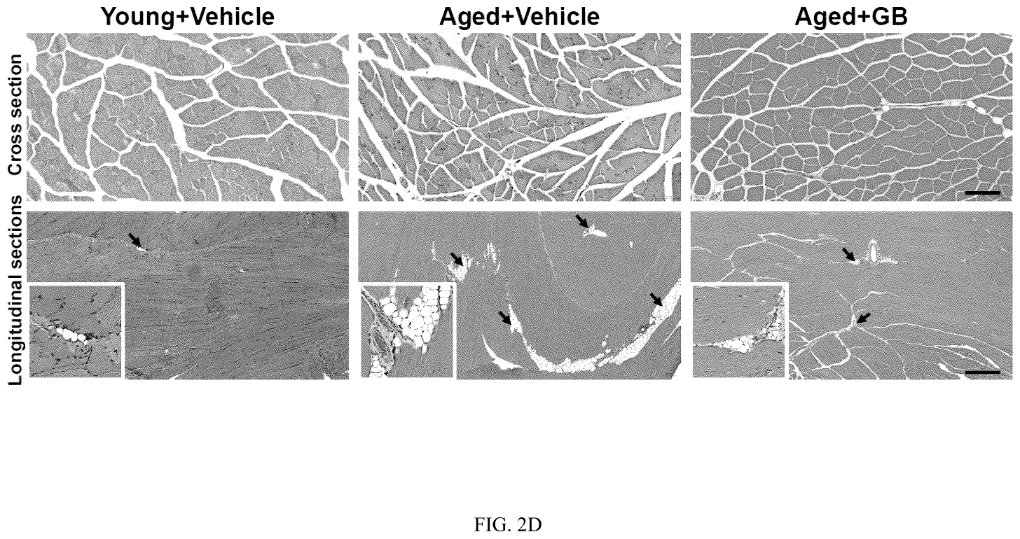

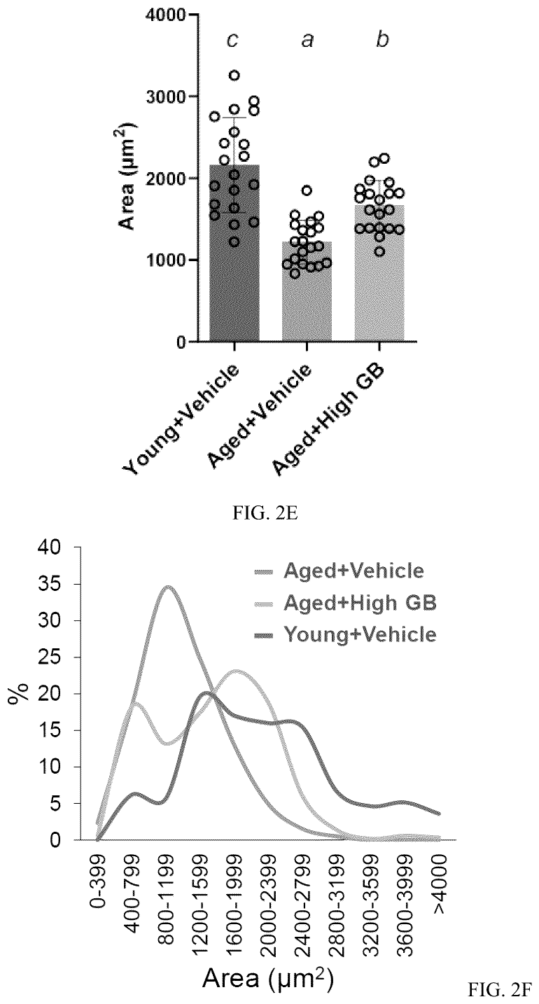

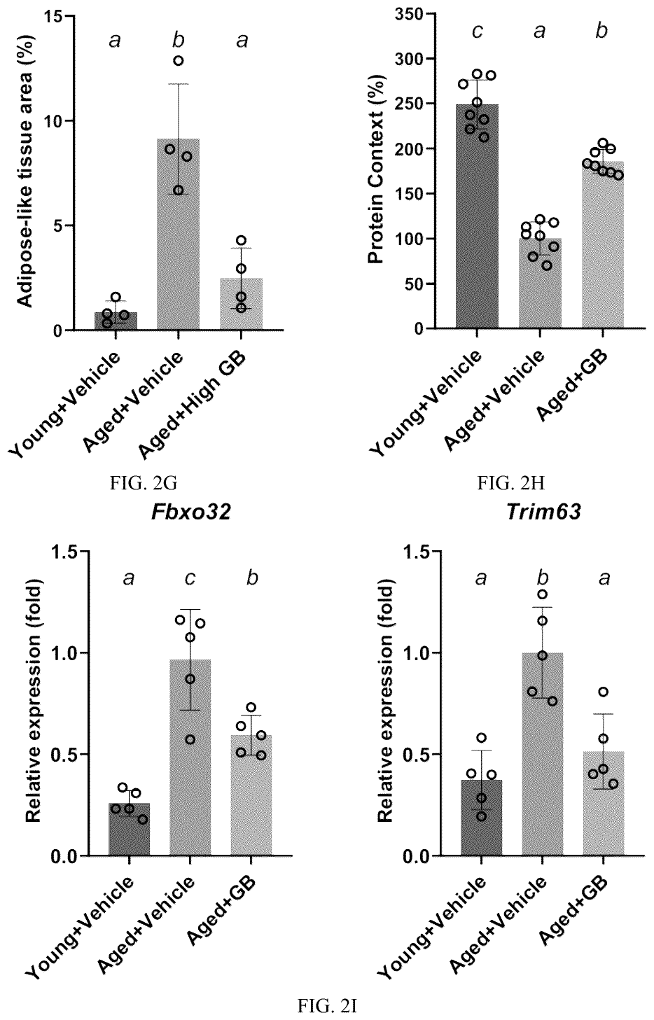

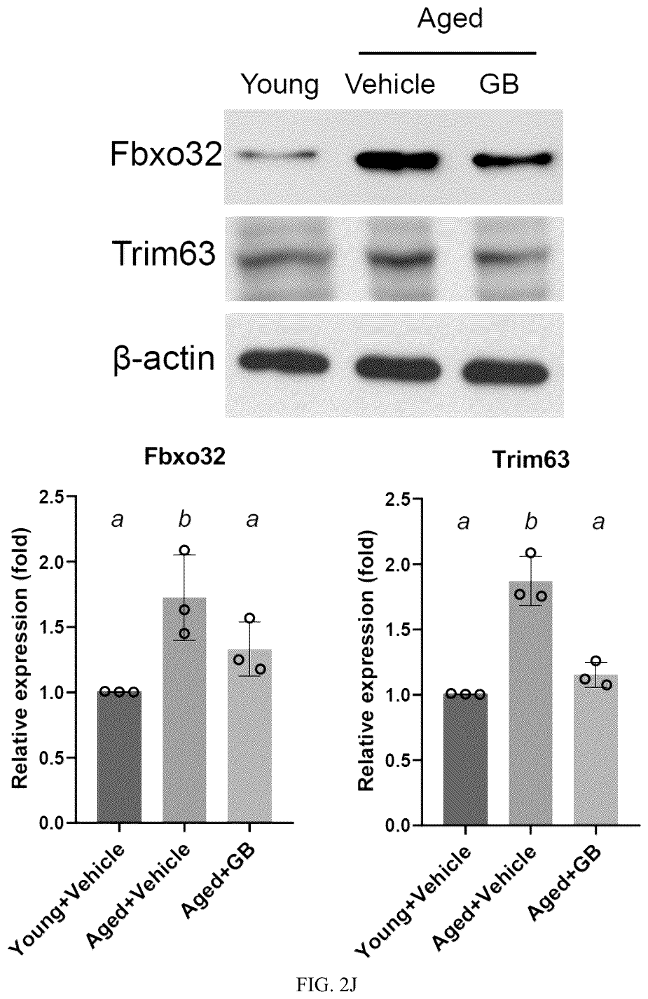

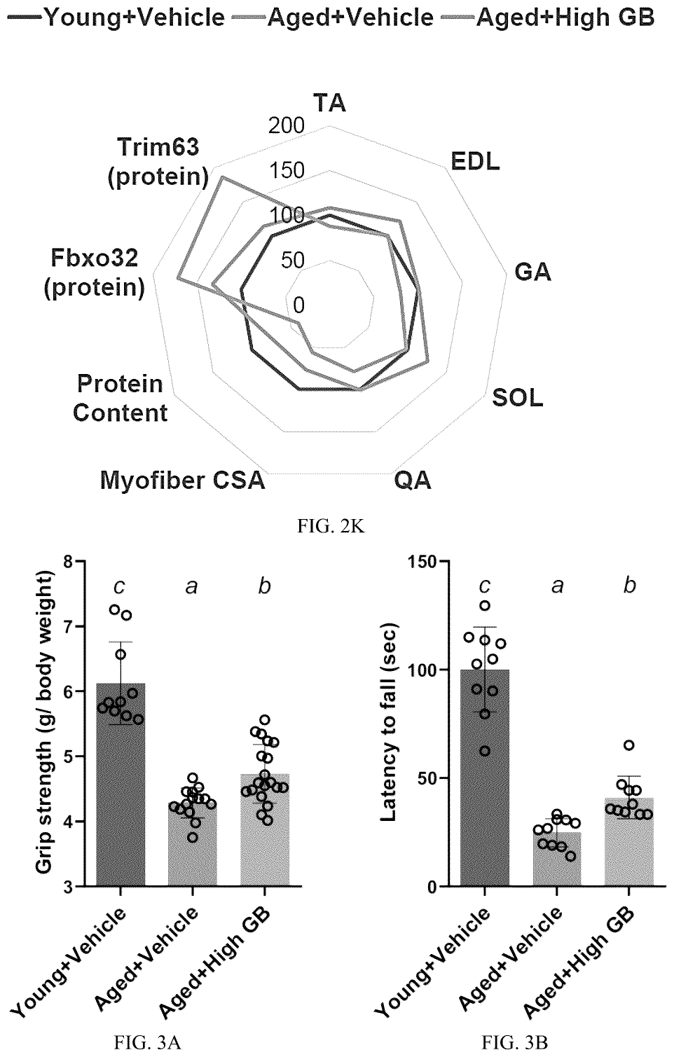

To investigate whether GB improves healthspan, we administered GB to 20-month-old aged mice, equivalent to 70-80-year-old humans, which is more relevant to potential clinical applications than intervention starting from young adulthood (Wang, S., Lai, X., Deng, Y. & Song, Y. Correlation between mouse age and human age in anti-tumor research: Significance and method establishment. Life Sci 242, 117242, doi: 10.1016/j.lfs.2019.117242 (2020)). After GB administration for two months, we assessed the healthspan domains in terms of sarcopenia, physical activity, metabolic health, frailty index, and inflammation (FIG. 2A) according to the criteria in clinical practice (Shamliyan, T., Talley, K. M., Ramakrishnan, R. & Kane, R. L. Association of frailty with survival: a systematic literature review. Ageing Res Rev 12, 719-736, doi: 10.1016/j.arr.2012.03.001 (2013)). As expected, circulating osteocalcin levels were decreased in aged mice, while GB administration increased osteocalcin levels (FIG. 2B, p<0.0001) toward the levels of those in young mice. Anatomical and histological observation demonstrated that, with aging, mice exhibited significant declines in skeletal muscle-to-body ratio (FIG. 2C), myofiber CSA (FIGS. 2D to 2F, p<0.0001) and increases in intermuscular adipose tissue (FIG. 2G, p=0.0003) which are all typical features of degenerative muscle in aged mice and aged humans and are associated with increased risk of morbidity in elderly . In line with muscle loss, aging led to a decreased in total protein content (FIG. 2H, p<0.0001) in skeletal muscle with upregulation of muscle atrophy markers, Fbxo32 and Trim63, at transcript (FIG. 2K, p<0.0001 and=0.0005, respectively) and protein levels. (FIG. 2L, p=0.0182 and 0.0003, respectively). Compared with vehicle-aged mice, GB administration significantly increased the skeletal muscle-to-body ratio (FIG. 2C) and myofiber CSA (FIGS. 2D to 2E, p=0.0032), and was associated with a shift toward proportion of larger fibers in the myofiber size-distribution curve (FIG. 2F) in aged mice. GB reversed the infiltration of adipose-like tissues by aging (FIGS. 2D and 2G, p=0.0012). GB administration increased the total protein content (FIG. 2H, p<0.0001) in aged skeletal muscle along with significant downregulation of the expression levels of Fbxo32 and Trim63 (FIGS. 21 to 2J, p=0.0078 and 0.0182 for Fbxo32, p =0.0039 and 0.0003 for Trim63, mRNA and protein, respectively), which were comparble to those of young mice. GB administration reversed aging-related muscle wasting (FIG. 2K).

### Example 3 GB Improves Physical Activity in Aged Mice

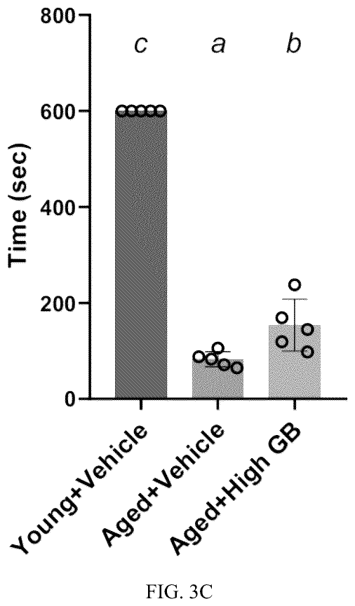

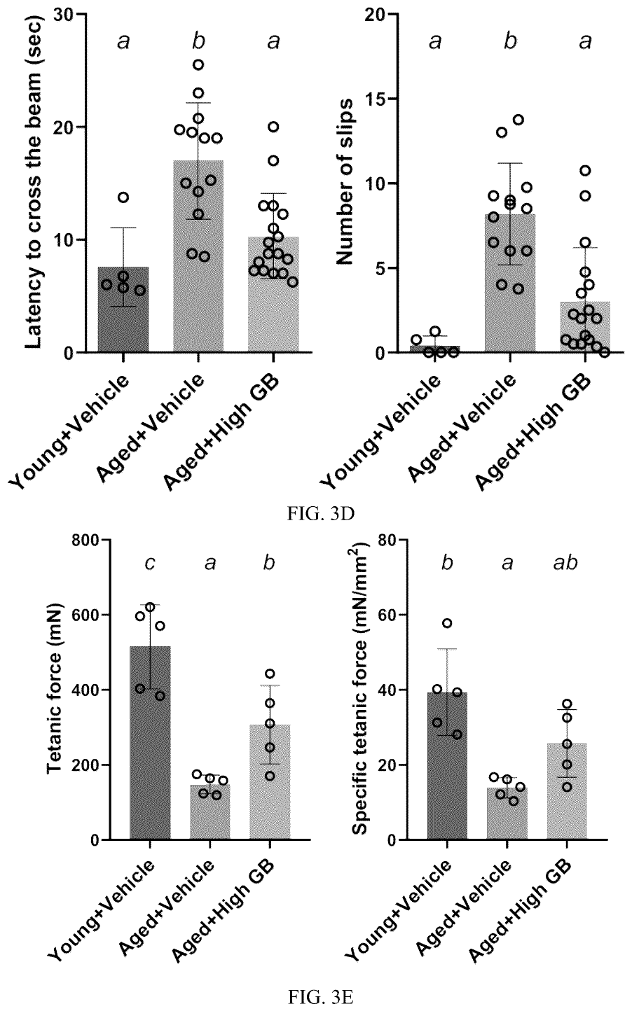

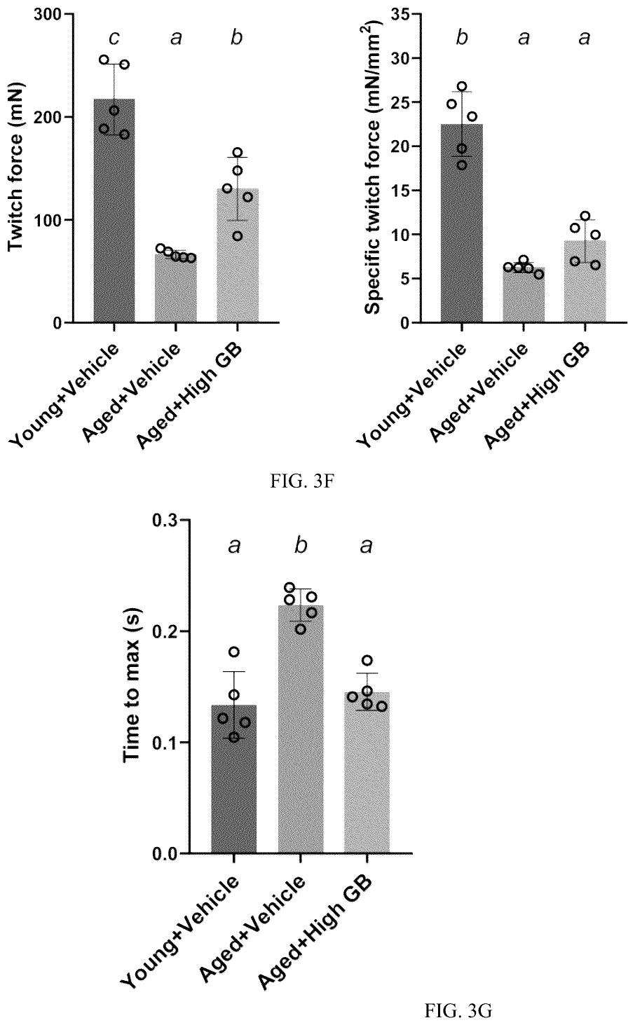

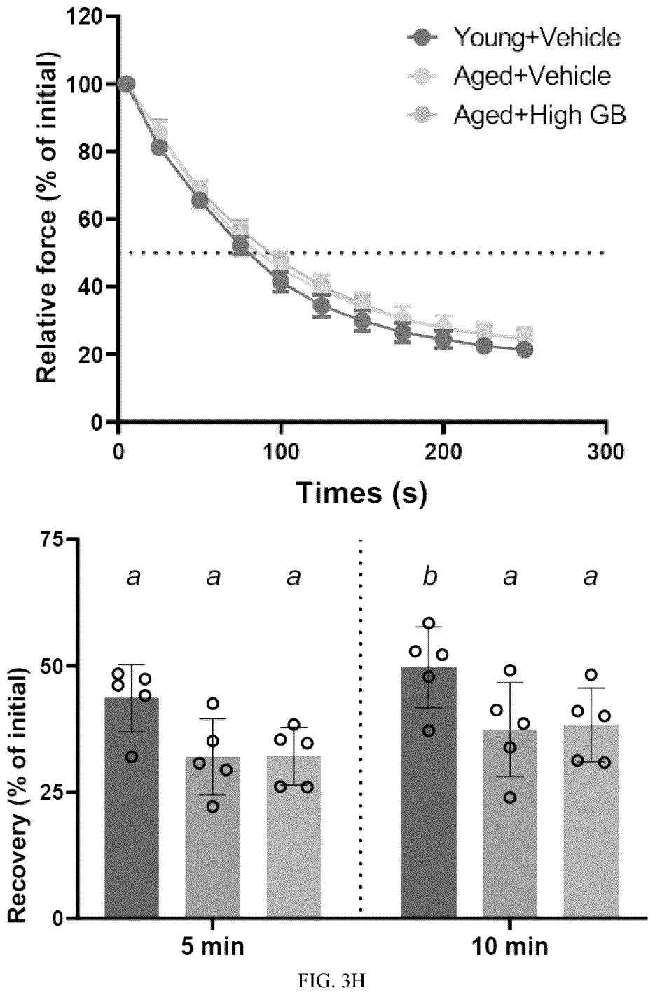

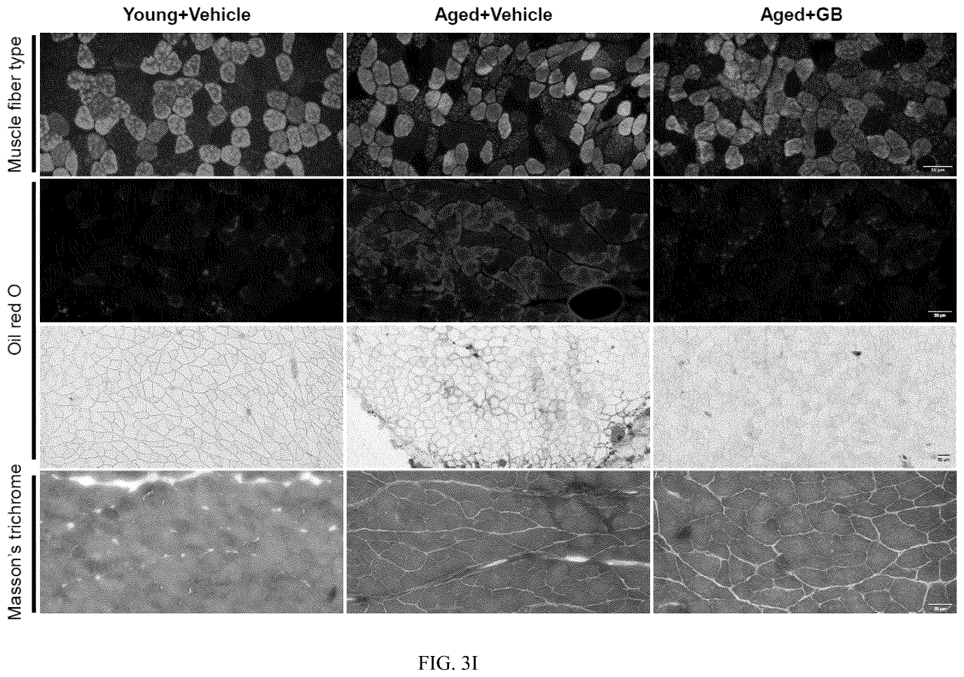

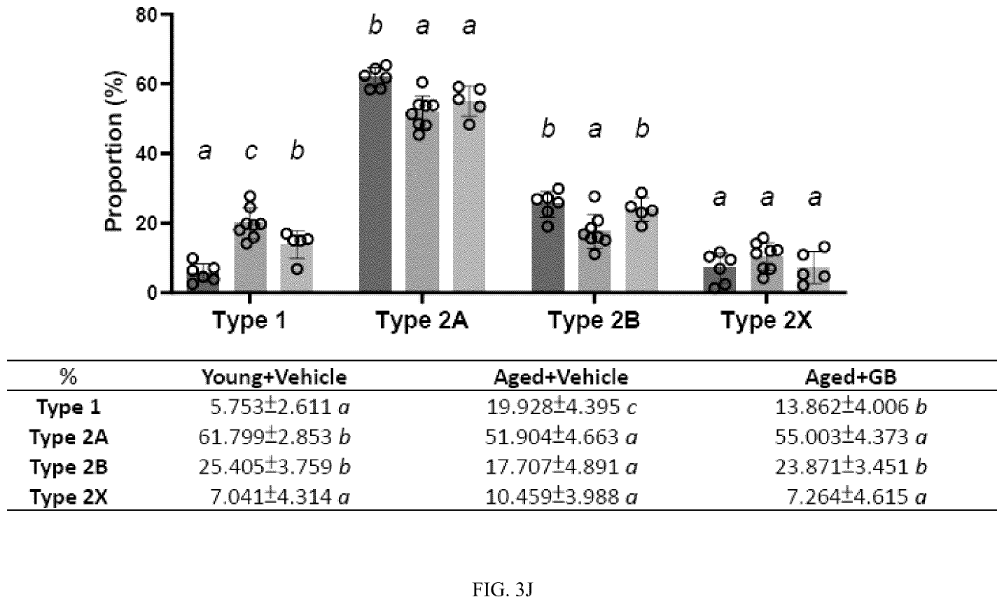

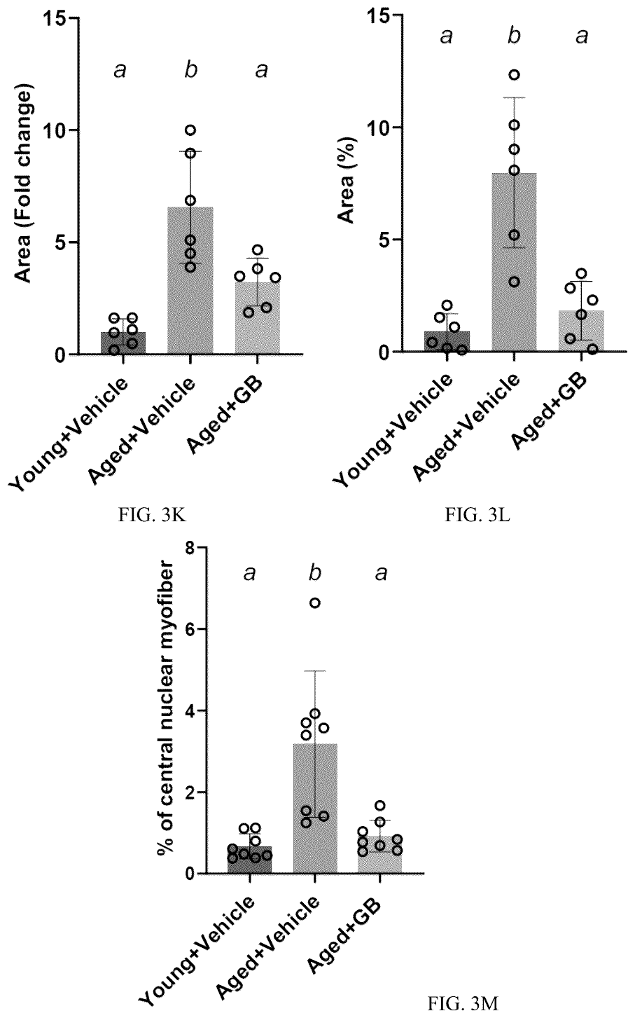

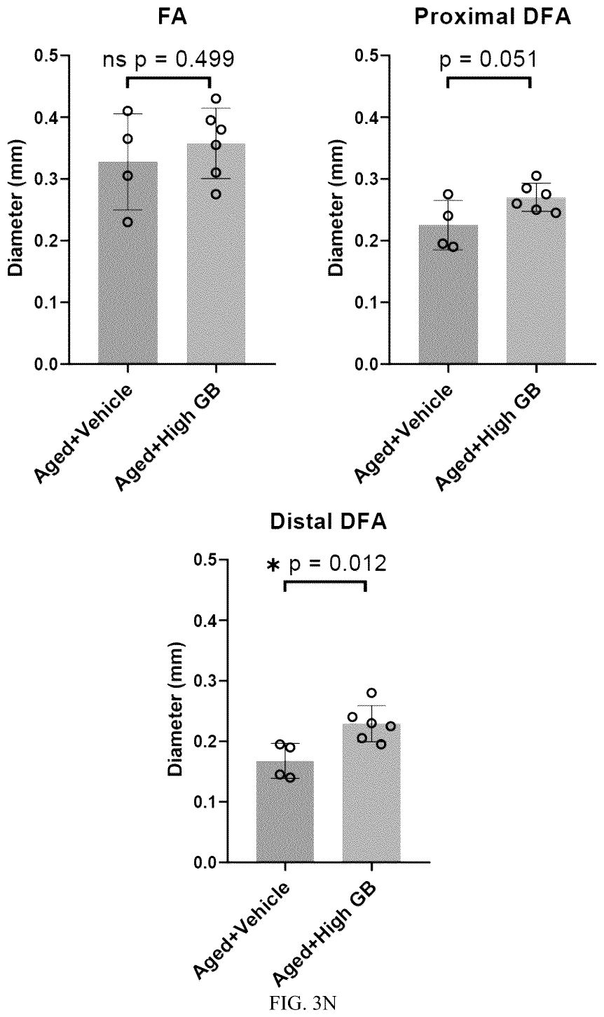

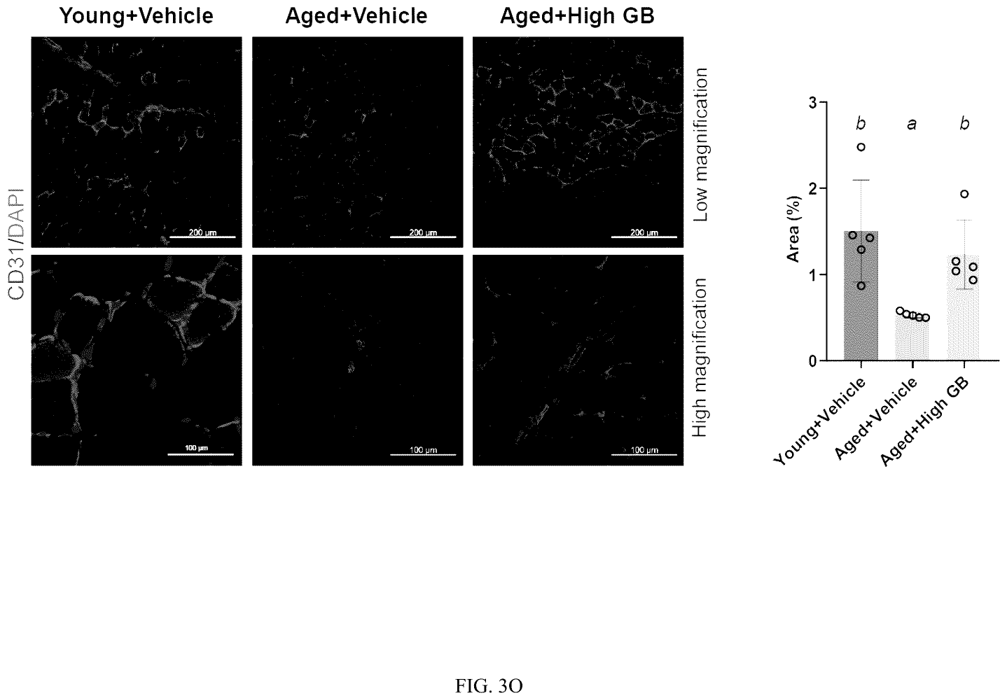

Next, we investigated the effect of GB on functional healthspan domains related to neuromuscular and physical performance. Compared with the young mice, aged mice exhibited lowered forelimb grip strength (FIG. 3A, p<0.0001) and exercise capacity (FIG. 3B, p<0.0001), hanging ability (FIG. 3C, p<0.0001), and gross motor coordination as revealed by increases in the duration to cross balance beam (FIG. 3D, p<0.001) and the number of slips (FIG. 3D, p<0.0001). Compared with vehicle-aged mice, GB-aged mice exhibited higher grip strength (FIG. 3I, p<0.01), and prolonged duration on the rotarod and hanging (FIGS. 3B to 3C, p<0.05 and<0.0001, respectively). In the balance beam test, GB also decreased the latency to cross (FIG. 3D, p<0.001) and reduced the number of foot slips (FIG. 3D, p<0.001), and the improvements of motor coordination were comparable to those of young mice.

Additionally, ex vivo muscle contraction tests were used to assess force properties. Vehicle-aged muscle exhibited aimpaired contractility, such as lower absolute and normalized tetanic force and twitch force, and delayed time to maximum force (FIGS. 3E to 3G, p<0.0001 for all parameters). GB administration in aged mice significantly enhanced those absolute tetanic force twitch force (FIGS. 3E to 3F, p=0.0404 and 0.0071, respectively), with a modest increased specific force (normalized to muscle mass) (FIGS. 3E to 3F, p=0.1161 and 0.1935, respectively). GB administration reversed the increased time to maximum force to the youth levels (FIG. 3G, p=0.0003). During the sustained and intermittent isometric contractive stimulation, aged muscle exhibited higher fatigue resistance but lower recovery rate than young mice (FIG. 3H, p=0.0461 at ten minutes), which is consistent with the observations in elderly humans. GB administration did not alter fatigue resistance or recovery rate in aged mice (FIG. 3H, p=0.99 at five and ten minutes). The muscular fiber-type switching, lipid infiltration, collagen deposition and spontaneous central myonucleus are associated with declination of muscle contraction and physical activity during aging. Aging increased the proportion of type 1 myofibers and decreased 2A and 2B myofibers. GB partially reversed the aging-related changes in fiber-type switching, in particular type 1 and 2B myofibers (FIG. 3I upper panel and 3J). GB administration in aged mice reduced the aging-related increase in intramuscular lipid infiltration (FIG. 3I middle panel and 3K, p=0.01), collagen deposition (FIG. 3I bottom panel and 3L, p=0.0004), and the number of central myonuclei in aged myofibers (FIG. 3M, p=0.0011). Additionally, aging-associated rarefaction of the muscular vessel network lowers the accessibility of oxygen, endocrine, and nutrients to muscles that diminish muscle protein synthesis, contractile force and eventually aggravate sarcopenia with aging. We found that GB dramatically enlarges the deep femoral artery (FIG. 3N, p=0.012) and capillary density (FIG. 3O, p=0.0492) in aged mice. Consequently, our results suggest that GB improved skeletal muscle mass, myofiber type proportions, intramuscular lipid accumulation, collagen deposition, musculovascular network to improve physical performance in aged mice toward youthful levels.

We then confirmed whether GB also improved muscle wasting and physical activity in estrogen-deficient mice. Compared with the non-OVX mice, OVX mice exhibited a significantly lower skeletal muscle mass, grip strength, exercise capacity and gross motor performance. Consistent with the results we observed in the aging model, GB administration dose-dependently restored skeletal muscle mass and myofiber CSA. High-dose GB administration improved forelimb grip strength and fully reversed the decline of motor performance. Of note, high-dose GB administration almost fully restored muscle wasting and physical activity in estrogen-deficient mice. In conclusion, our data showed that GB administration promoted muscle mass and physical activities in three different models.

### Example 4 The Effects of GB on Aging-Related Changes in Metabolic Health

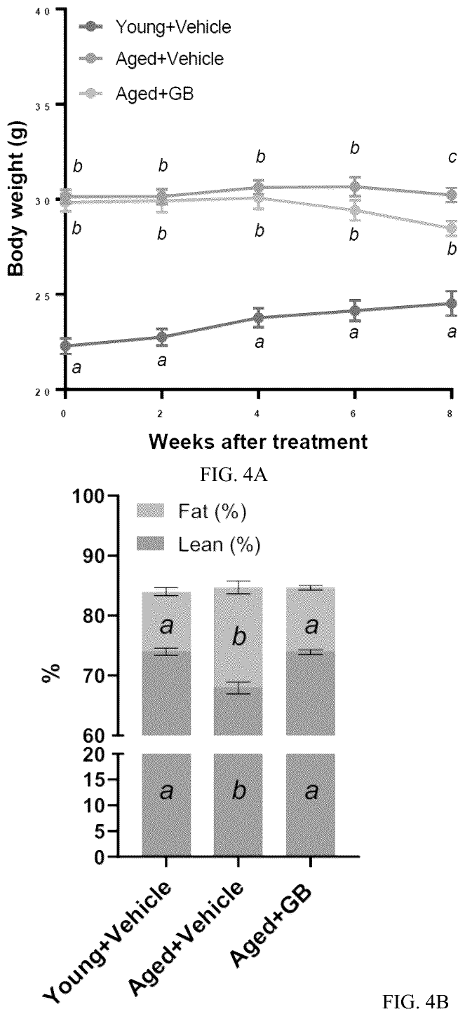

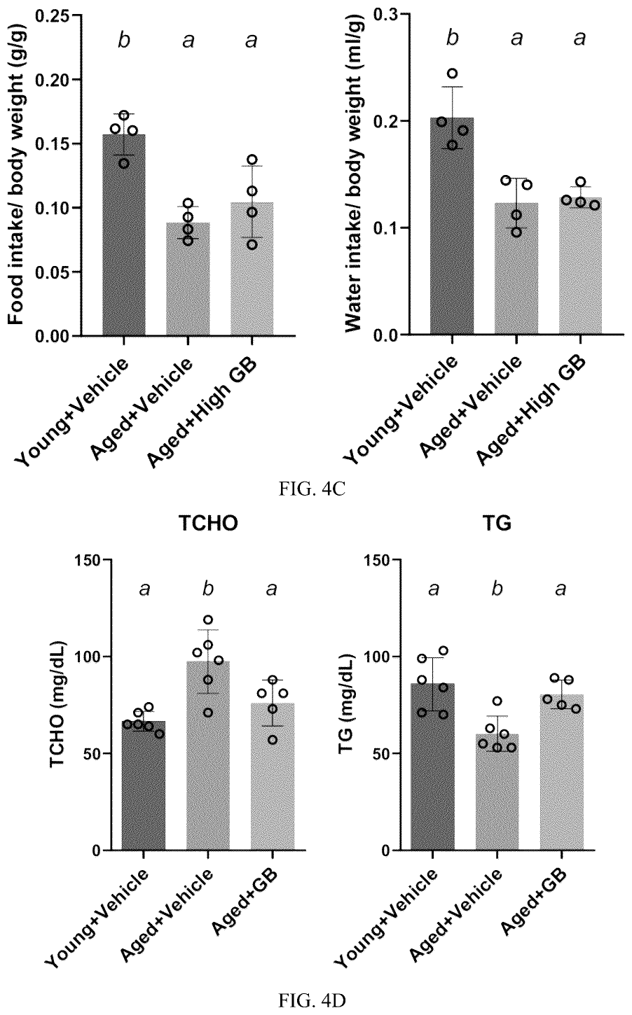

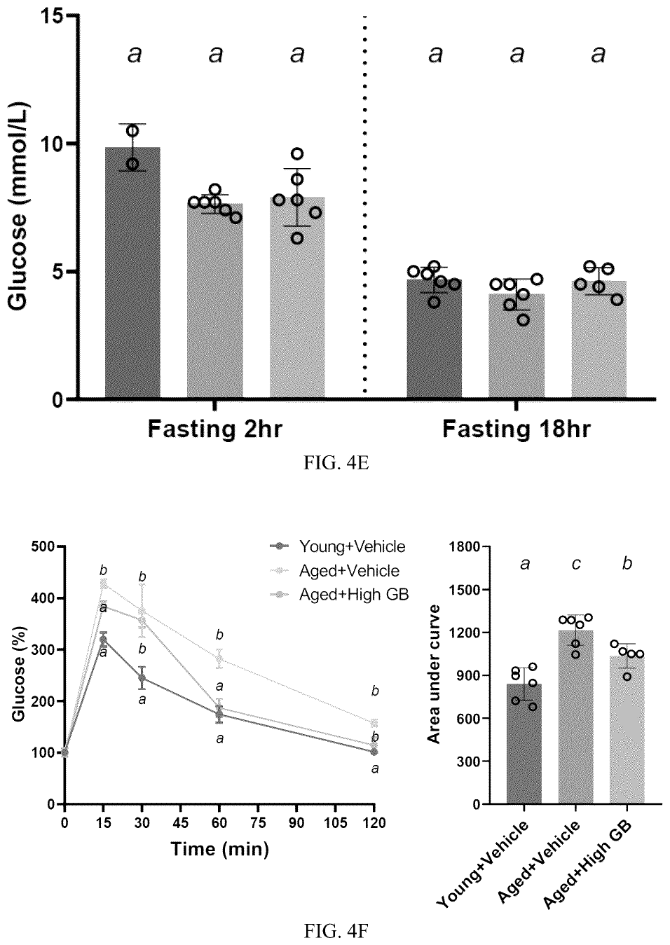

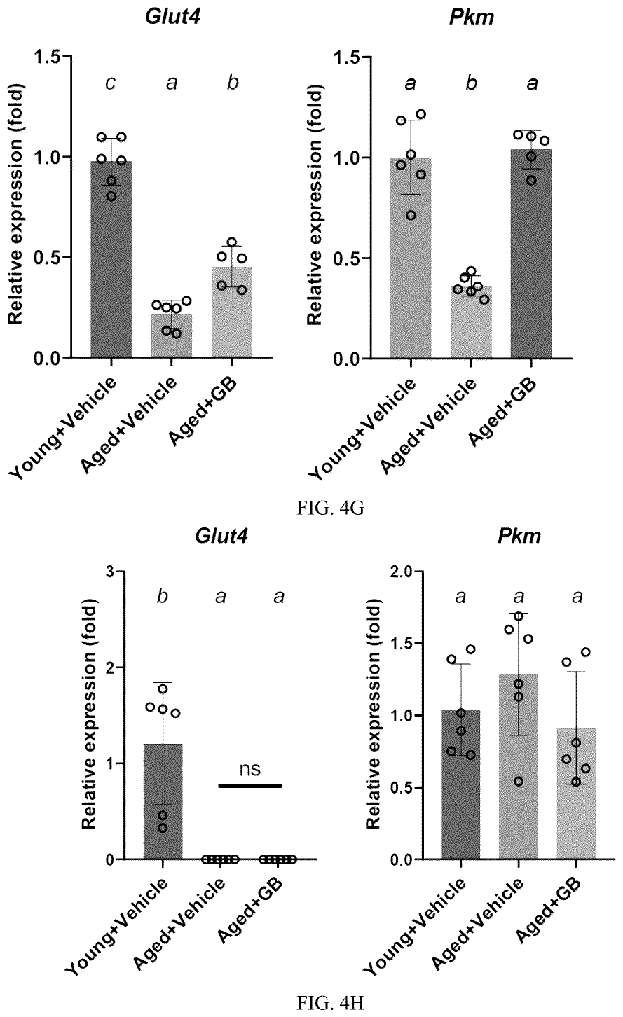

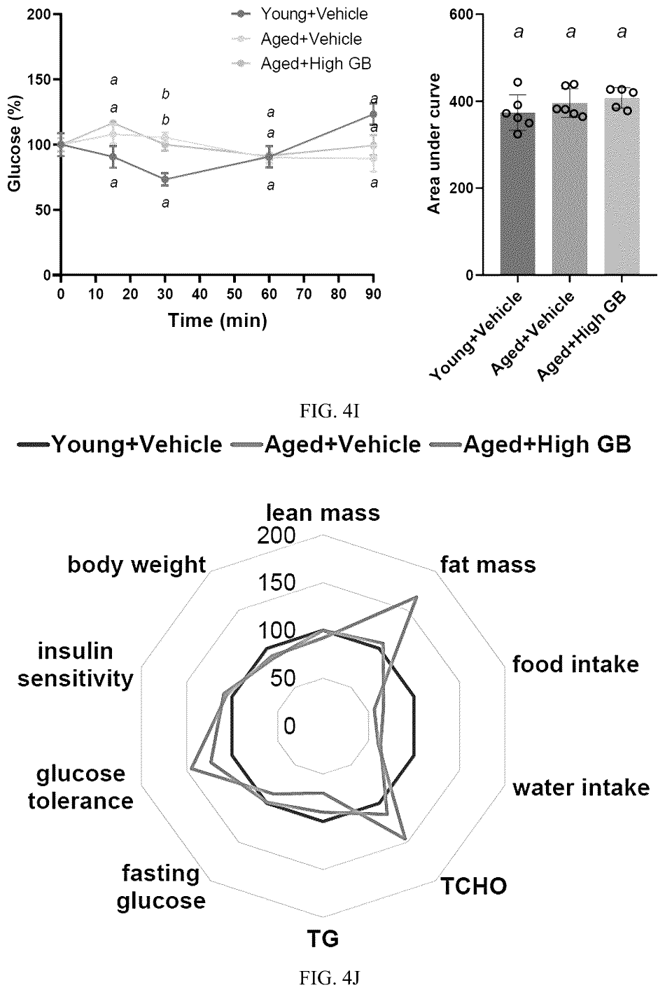

To assess metabolic health, we measured body weight, body composition, food/water intake, glucose tolerance and insulin sensitivity in aged mice. Body weight and body composition have been deemed as predictors of metabolic health and longevity in mice. Reduced body weight alongside increased lean mass-to-fat ratio are reportedly associated with healthspan and longevity in long-lived mouse models (Martin-Montalvo, A. et al. Metformin improves healthspan and lifespan in mice. Nat Commun 4, 2192, doi: 10.1038/ncomms3192 (2013)) and humans (Srikanthan, P. & Karlamangla, A. S. Muscle mass index as a predictor of longevity in older adults. Am J Med 127, 547-553, doi: 10.1016/j.amjmed.2014.02.007 (2014)). During the period of administration, GB modestly but significantly reduced body weight progressively (FIG. 4A, 6% body weight reduction post-GB administration 2 months, p=0.0123) with a decrease in whole-body fat mass and an increase in lean mass (FIG. 4B, p<0.0001 and<0.0001, respectively), and the body compositions of GB-aged mice were comparable to those of young mice (Table S3). Food and water consumptions were not improved by GB administration compared with vehicle treatment (FIG. 4C, p=0.5127 and 0.9368, respectively). GB administration reversed the aging-related alteration in scrum triglyceride and total cholesterol levels (FIG. 4D, p=0.0424 and 0.0284, respectively). To examine whether GB administration affected glucoregulatory ability, we performed IPGTT and IPITT and measured the expression of glucose metabolism-related genes. Although there was no significant difference in the levels of fasting blood glucose among the three groups (FIG. 4E), vehicle-aged mice exhibited severe glucose intolerance (FIG. 4F, p<0.0001 in AUC) and lower glucose metabolism-related genes, glucose transporter 4 (Glut4) and pyruvate kinase muscle (Pkm), in skeletal muscle compared with young mice (FIG. 4G, p<0.0001). GB administration improved aging-related glucose intolerance (FIG. 4F, p=0.0322 in AUC) and restored the expression of Glut4 and Pkm in aged skeletal muscle (FIG. 4G, p=0.0035 and<0.0001, respectively), but not in aged liver (FIG. 4H). In contrast to the changes in glucose tolerance, there was no difference in insulin sensitivity among the three groups (FIG. 4I), which might be attributed to greater sensitivity to insulin in females. Taken together, GB administration partially reverses aging-related changes in metabolic dysregulation independent of energy uptake/calorie restriction and insulin sensitivity (FIG. 4J).

### Example 5 GB Ameliorates Aging-Related Frailty in Mice

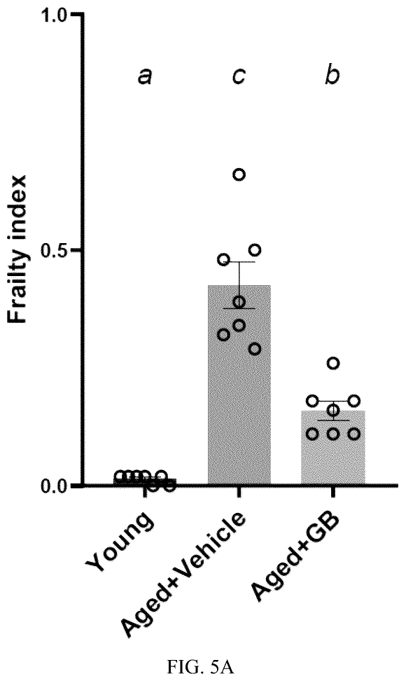

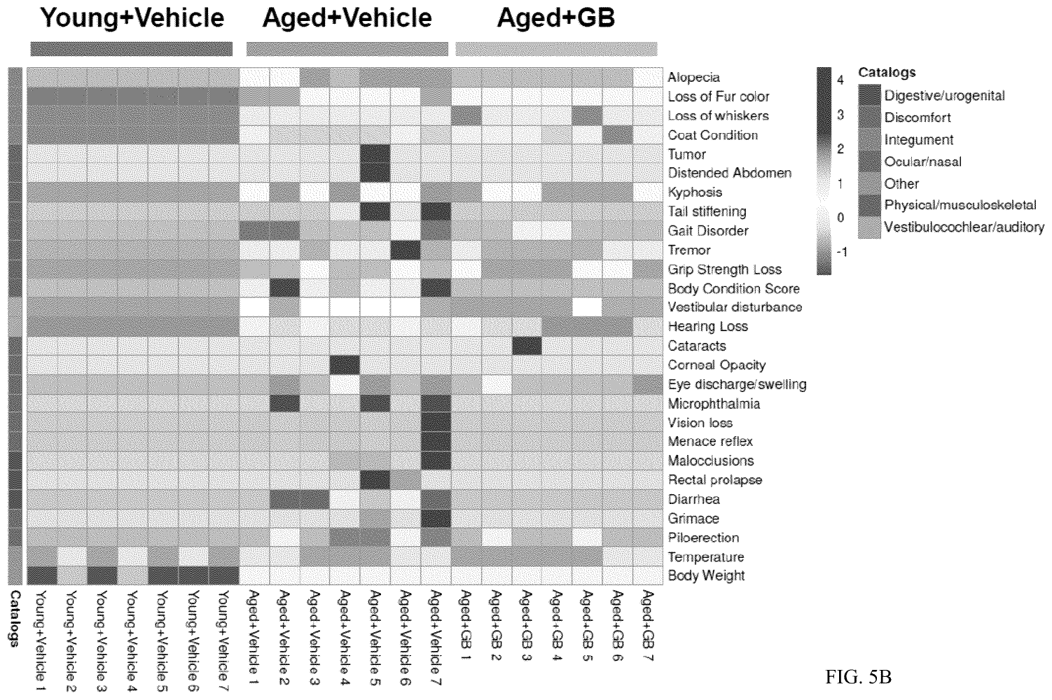

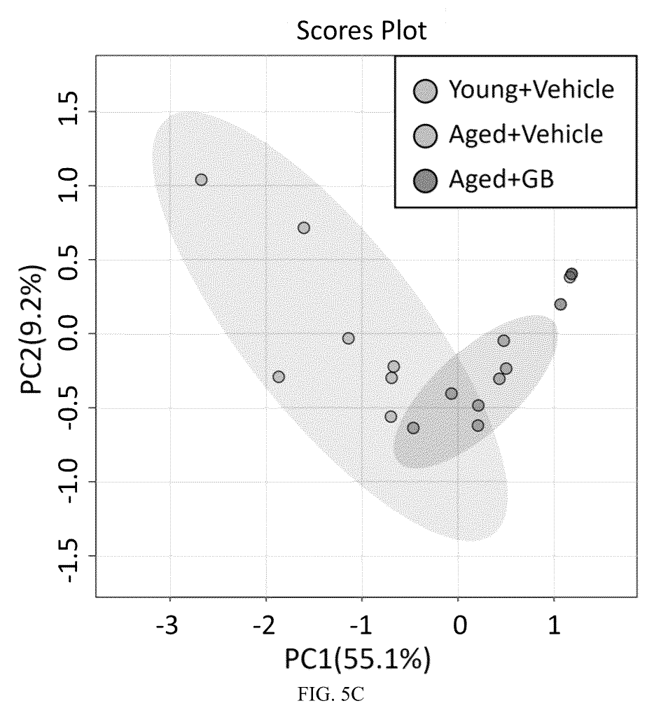

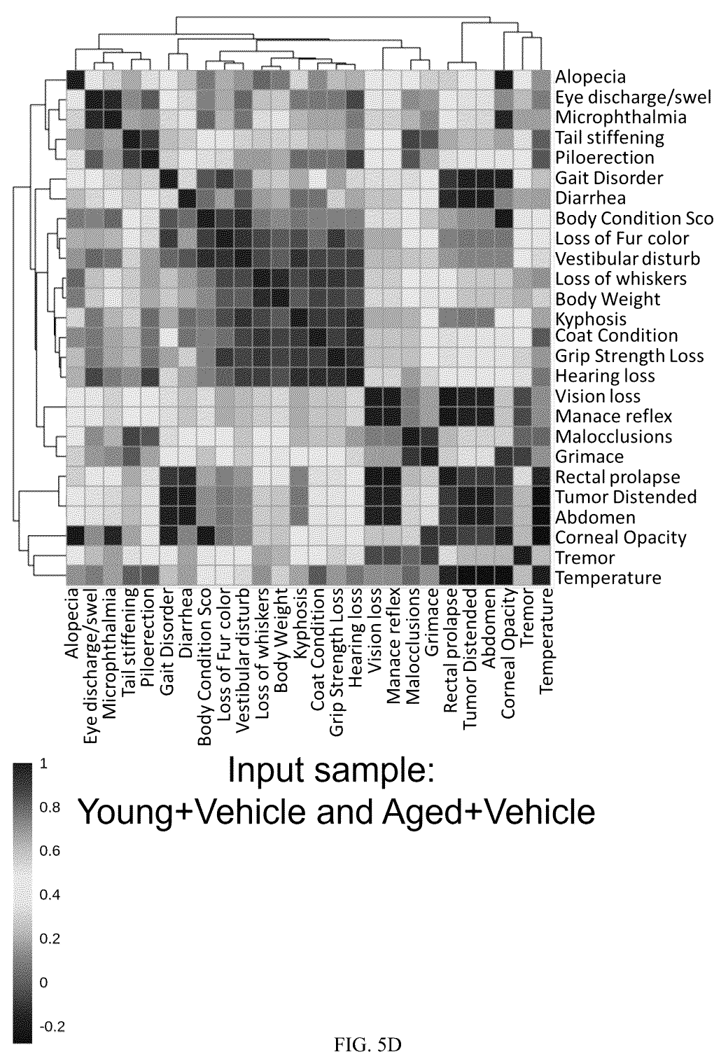

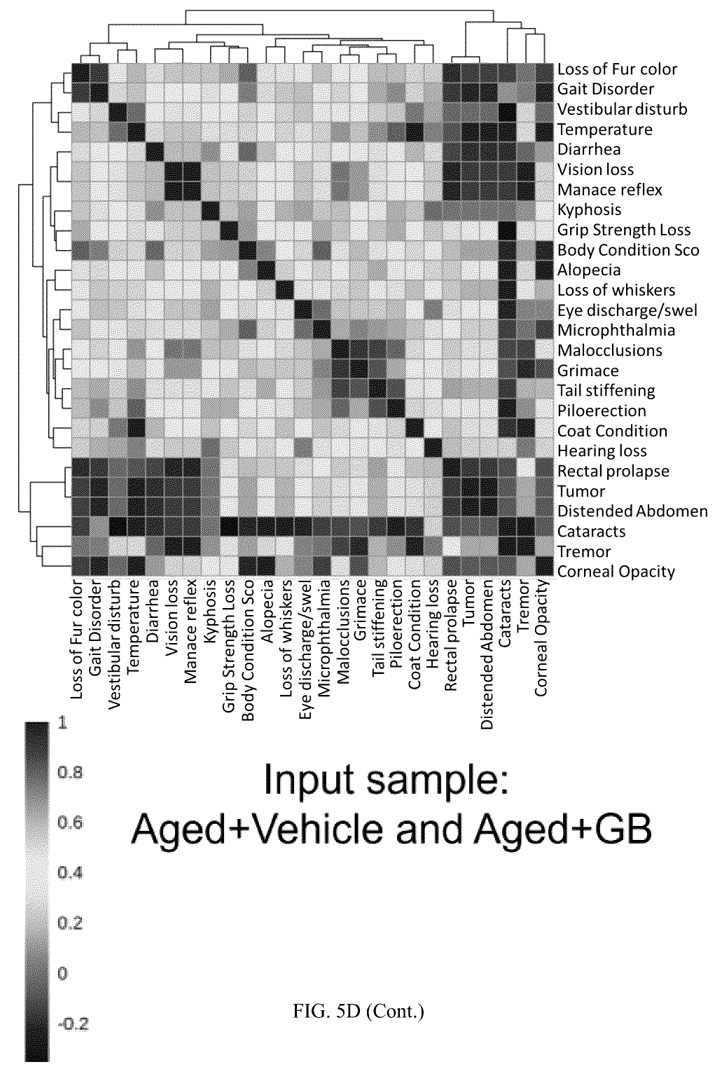

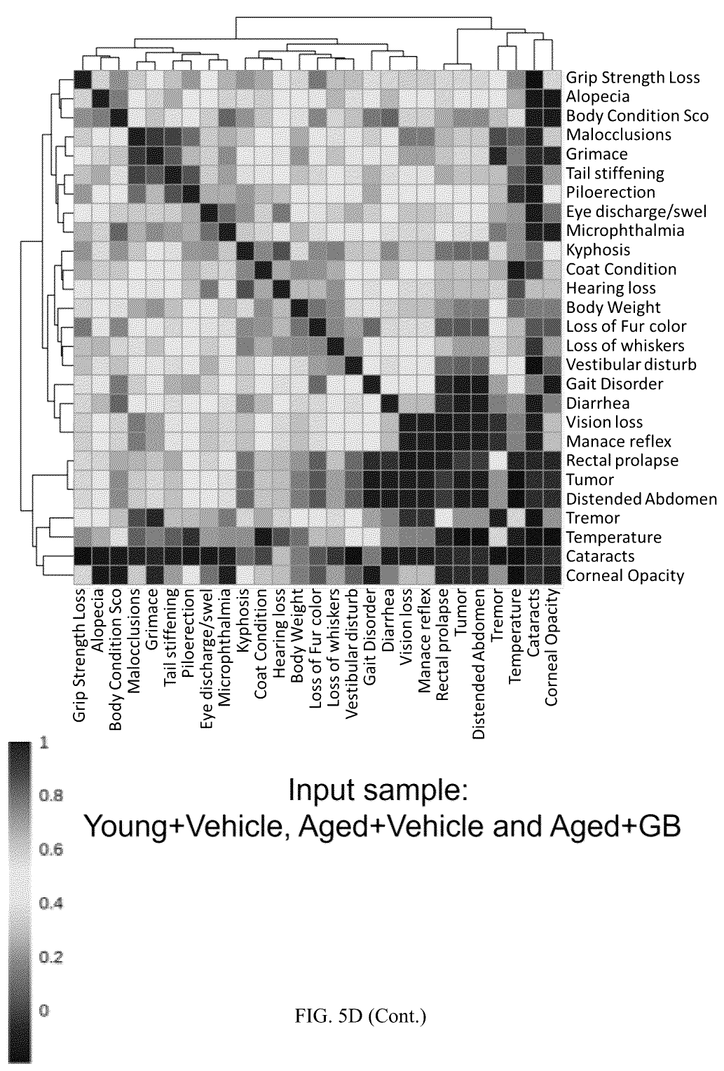

Frailty is an aging-related multidimensional state of adverse health decline, which is negatively correlated to lifespan and healthspan with aging. To evaluate the impact of GB administration on frailty, we applied a murine frailty index according to clinical-based measurement established by Whitehead et al. (Whitehead, J. C. et al. A clinical frailty index in aging mice: comparisons with frailty index data in humans. J Gerontol A Biol Sci Med Sci 69, 621-632, doi: 10.1093/gerona/glt136 (2014)), which has been widely utilized to assess the therapeutic efficacy of anti-aging interventions in murine (Palliyaguru, D. L., Moats, J. M., Di Germanio, C., Bernier, M. & de Cabo, R. Frailty index as a biomarker of lifespan and healthspan: Focus on pharmacological interventions. Mech Ageing Dev 180, 42-48, doi: 10.1016/j.mad.2019.03.005 (2019)). Compared with young mice, 22-month-old female aged mice showed a significantly higher average frailty index score (FIGS. 5A to 5B; Young, 0.01±0.01; Aged, 0.43±0.13, p<0.0001), even though variability existed within the aged group (FIGS. 5B to 5C). Among 31 frailty measurements, aging remarkably increased the incidence of 18 measurements majorly encompassing categories of the integument (significance in 4/5 items), physical/musculoskeletal (6/8), vestibulocochlear/auditory (2/2), ocular/nasal (3/7), digestive/urogenital (1/4), discomfort (1/2), and other (1/2), while eye discharge/swelling (p=0.0506) showed an increasing trend but did not reach statistical significance. GB administration significantly reduced aging-related frailty index score by 63.1% (FIG. 5A; GB, 0.16±0.05, p<0.0001) and PCA showed that GB-aged mice partially overlapped between vehicle-aged mice and young mice, indicating that GB ameliorated aging-related frailty (FIG. 5C). Indeed, GB reversed 13 aging-related frailty measurements (13/18) and modestly attenuated 3 measurements (3/18), thus positively impacting all measurements except hearing loss and body weight. In addition, we did not observe any deterioration of frailty in GB-aged mice as compared with vehicle-aged mice. Of note, correlation matrix constructed by either two groups or all three groups showed a highly positive correlation coefficient pattern, indicating GB systematically improved frailty without skewed preference (FIG. 5C). Our results indicated that GB administration for 2 months reversed aging-related frailty.

### Example 6 GB Modestly Modulates Aging-Associated Systemic Inflammation

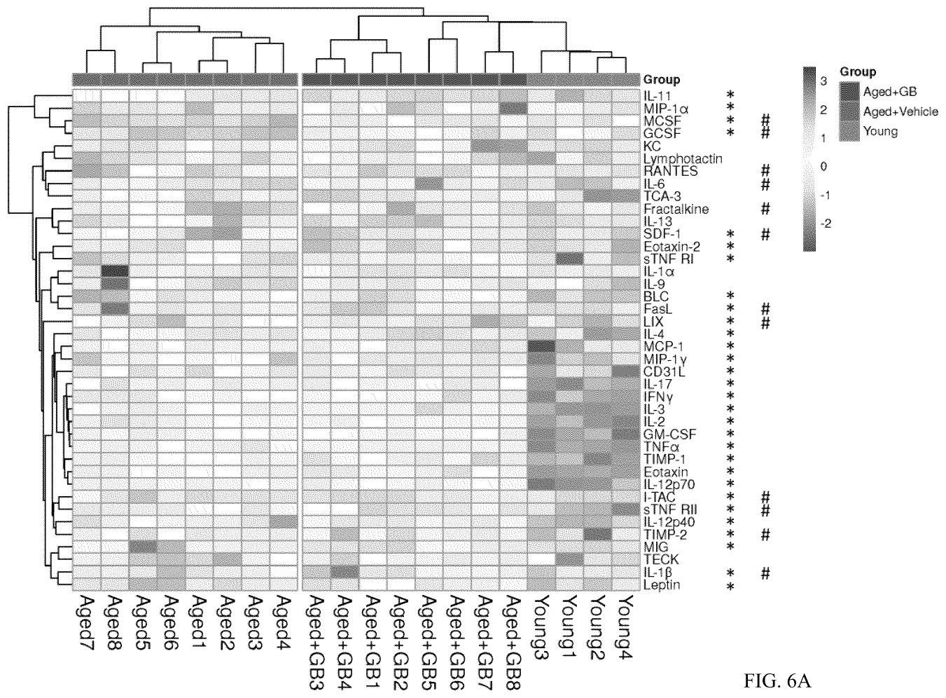

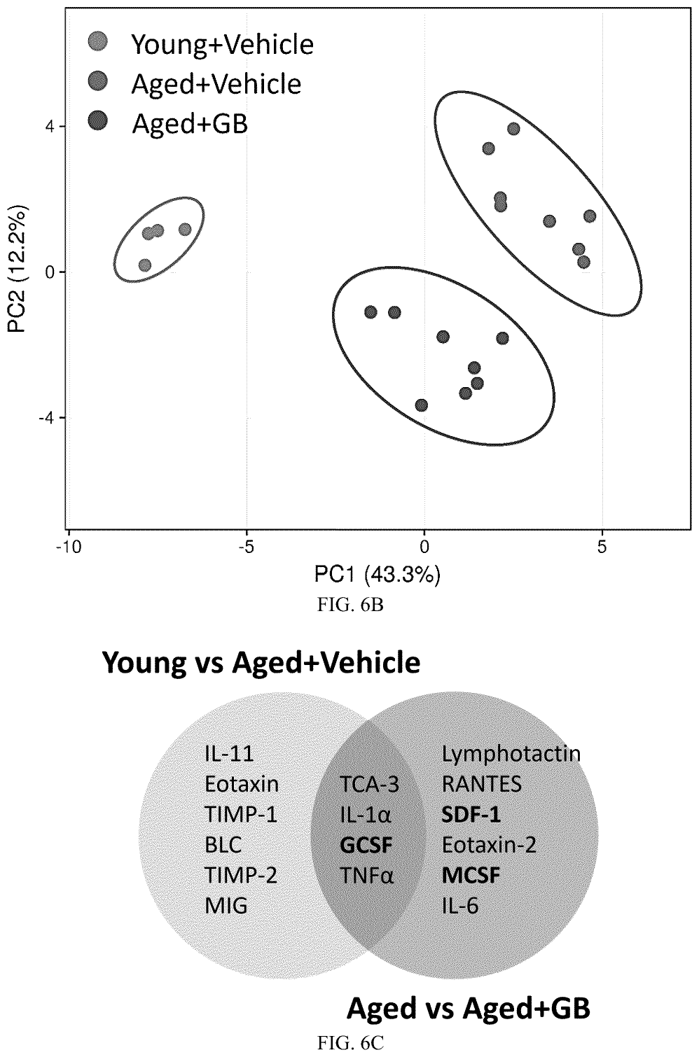

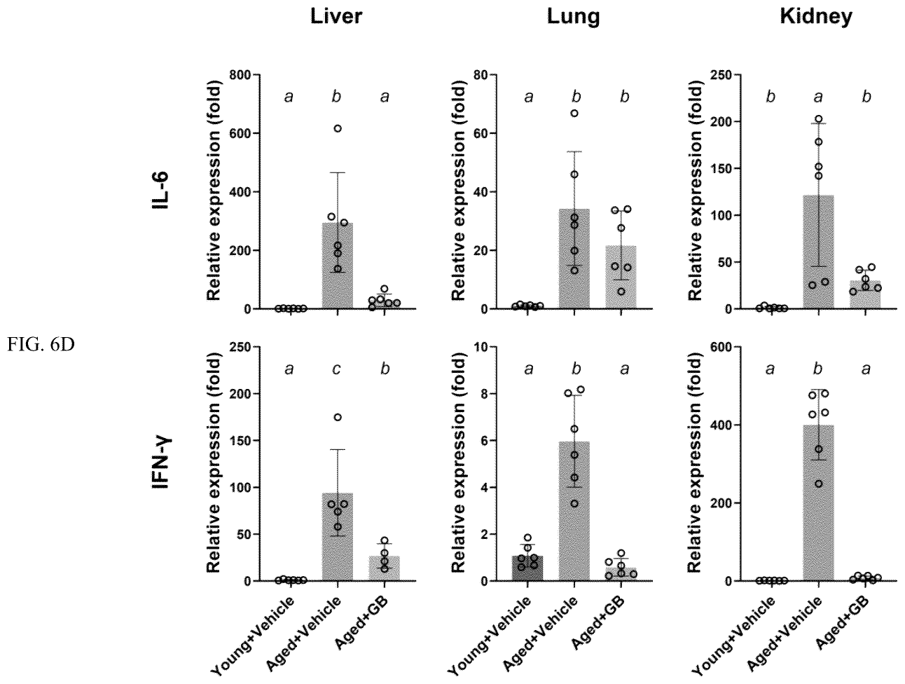

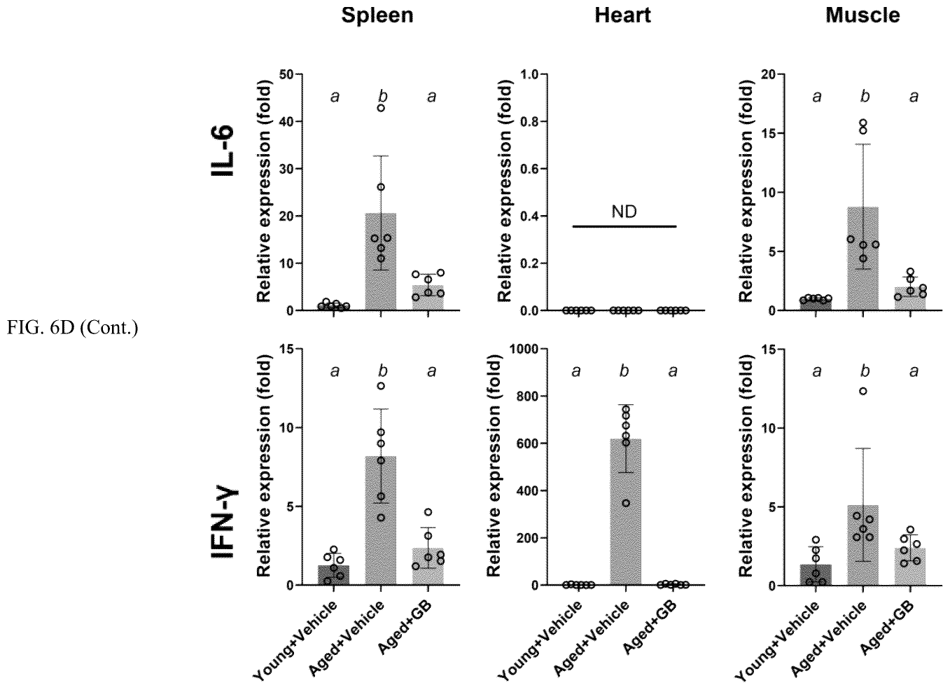

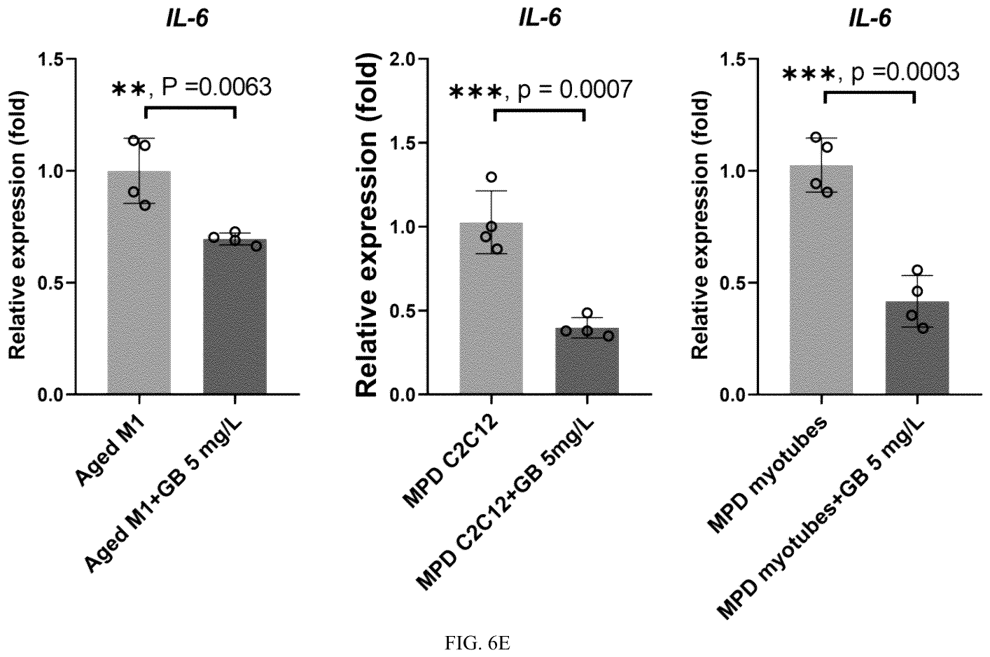

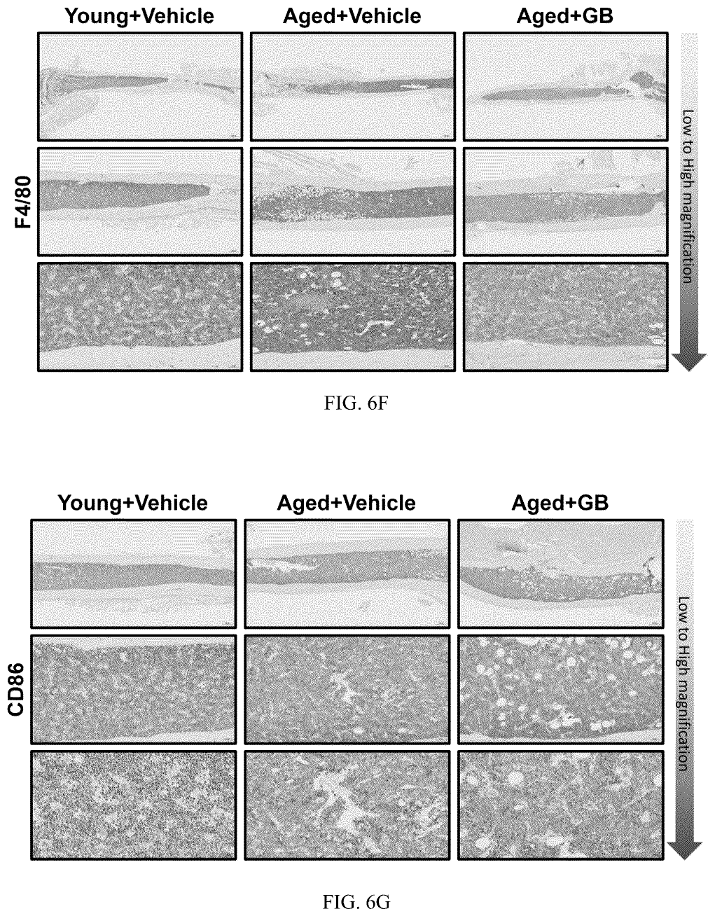

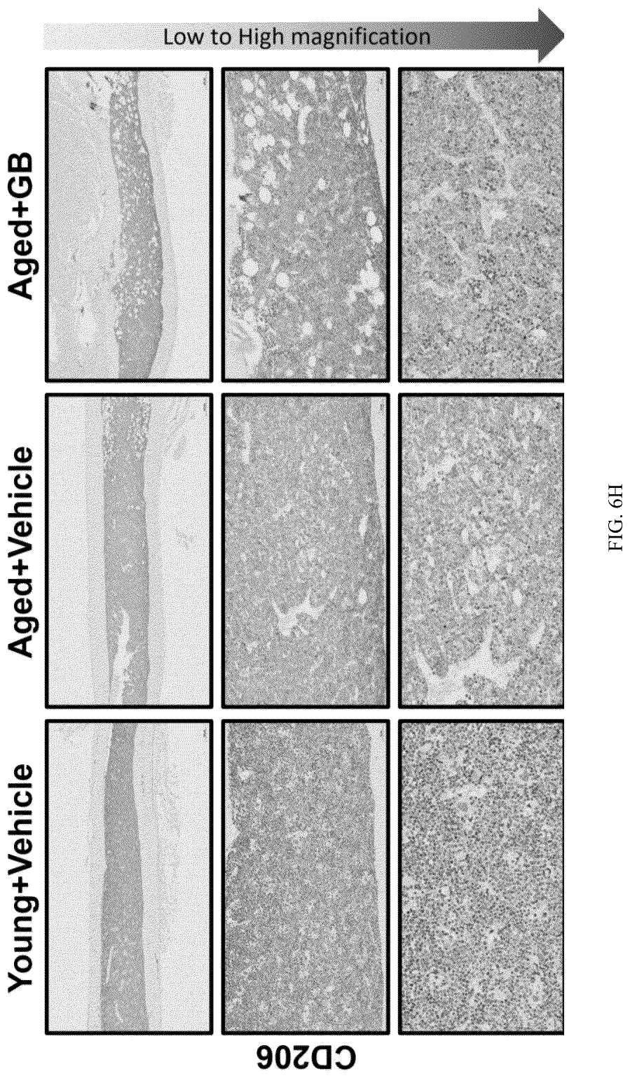

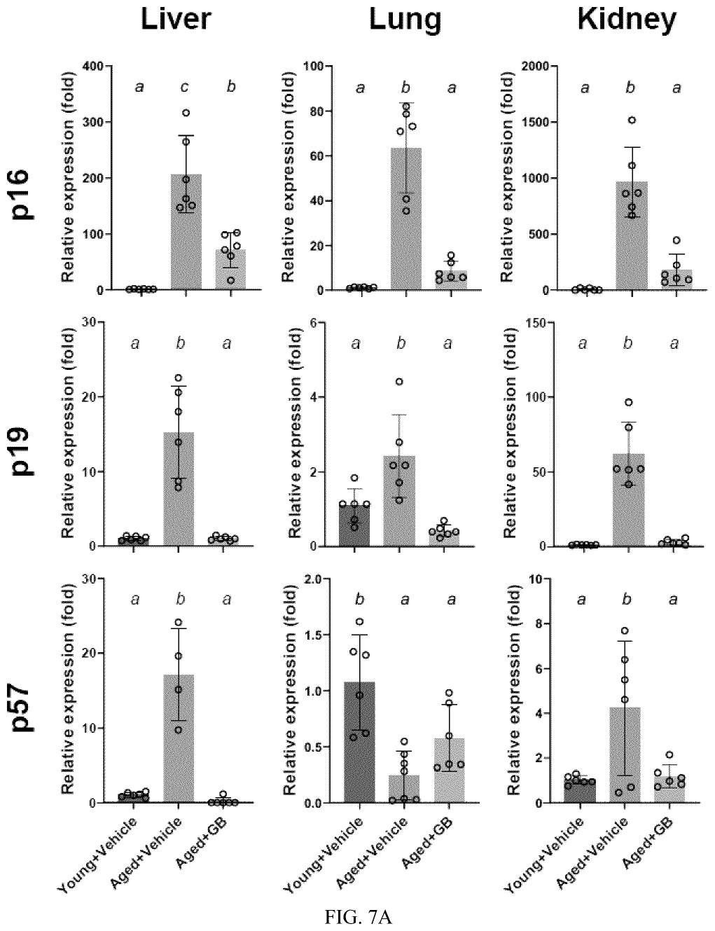

Inflammaging refers to aging-associated systemic, chronic, low-grade inflammation without infection, which substantially contributes to aging-related diseases and functional deterioration, such as metabolic syndrome, diabetes, frailty, and sarcopenia. A dramatic fluctuation of systemic inflammation during aging was revealed by a separated profile of inflammatory cytokines and chemokine between young and aged mice. In contrast, GB-aged and young groups tended to cluster together (FIG. 6A). A total of 30 of 40 circulating proteins we detected were significantly altered by aging, while GB administration significantly altered 12 proteins highly associated with aging-related profile. PCA analysis also showed a clear separation between inflammatory profiles among groups, and GB administration modestly reversed aging-related changes (FIG. 6B). Using FIT, a machine learning methodology, to extrapolate the animal results to the equivalent human condition, we showed that several human-relevant aging circulating proteins, such as TCA-3, IL-1α, GCSF, and TNFα, were altered by GB administration, indicating that those could be aging biomarkers or attractive druggable targets for anti-aging in humans (FIG. 6C).

The anti-inflammatory nature of GB was also confirmed by downregulated inflammatory cytokines, IL-6 and IFN-γ, in multiple tissues of aged mice (FIG. 6D) and in aged cells under culture conditions (FIG. 6E). In addition, aging-related accumulation of bone marrow F4/80+cells, the main pan marker of macrophages, was reversed by GB administration (FIG. 6F) along with a decrease in CD86⁺ pro-inflammatory M1 macrophages and an increased in CD206⁺ anti-inflammatory M2 macrophages (FIGS. 6G to 6H), indicating that GB shifted pro-inflammatory to anti-inflammatory state in aged mice.

### Example 7: GB Reduces Senescence Burden In Vivo and In Vitro

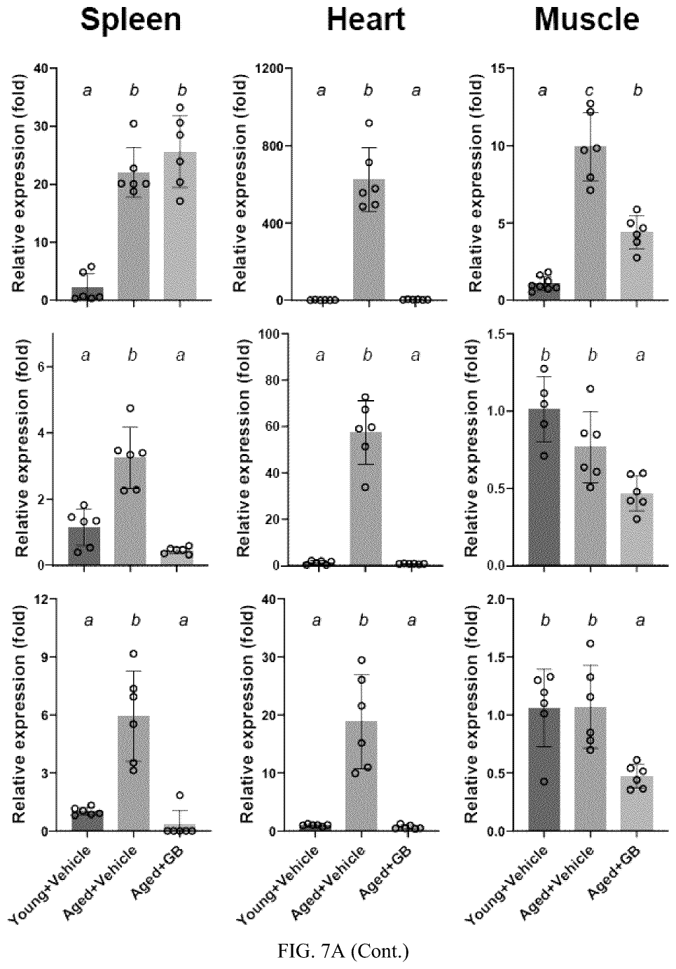

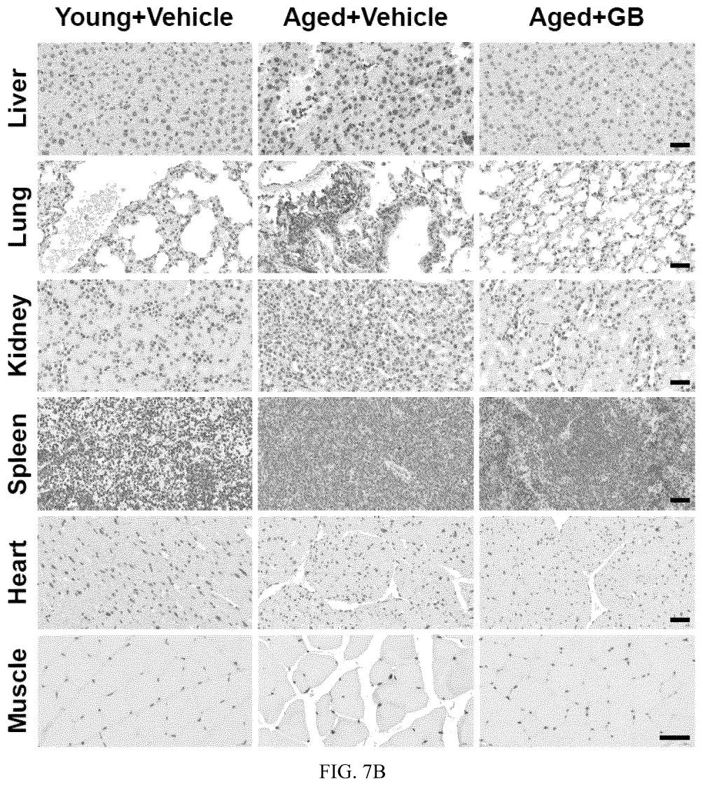

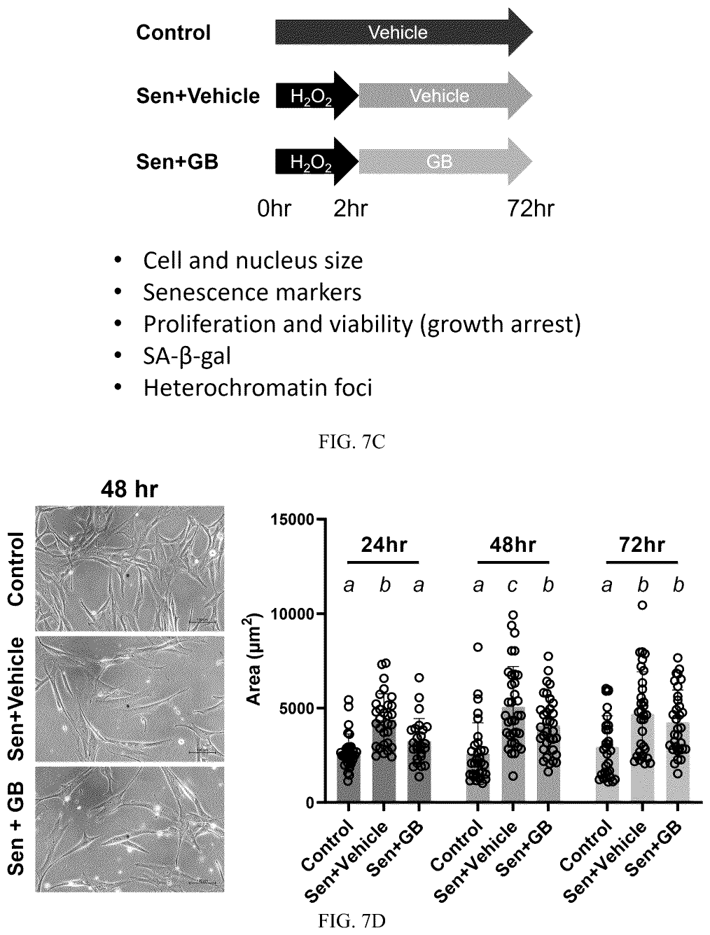

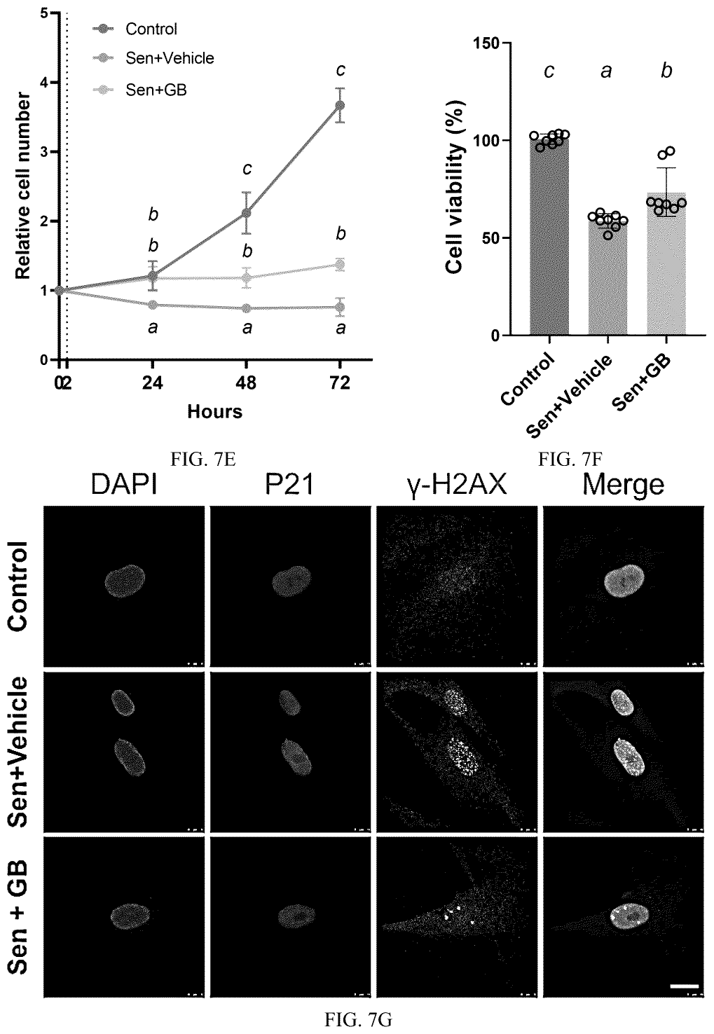

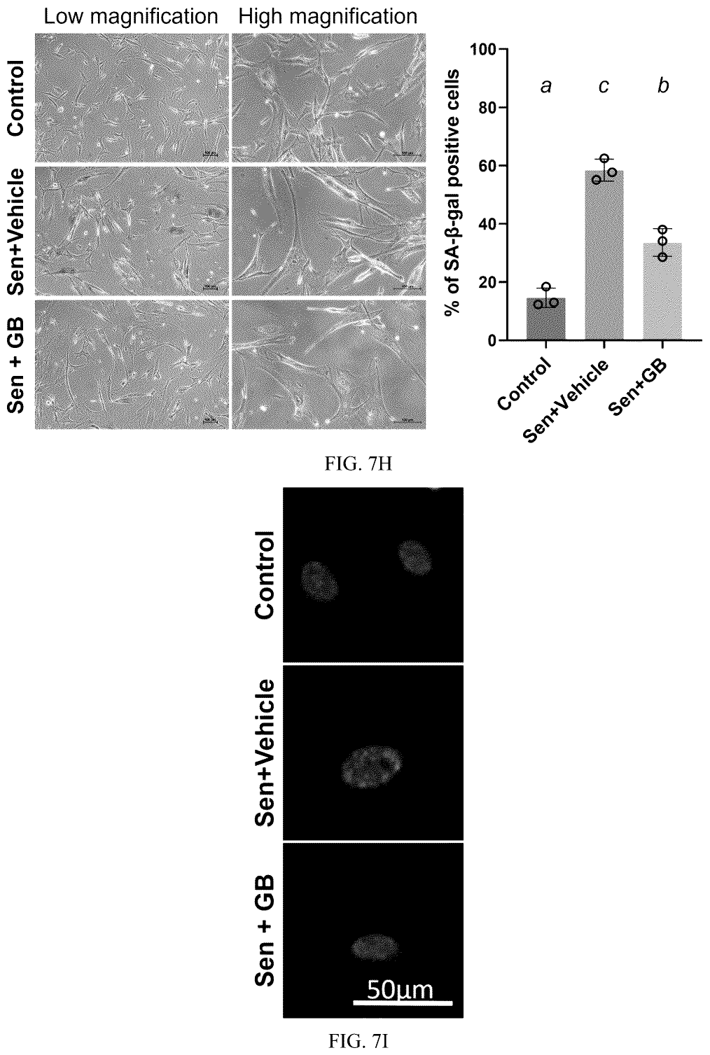

Senescent phenotypes play a causative role in aging and aging-related signature, and impair healthspan and shorten life expectancy (Di Micco, R., Krizhanovsky, V., Baker, D. & d'Adda di Fagagna, F. Cellular senescence in ageing: from mechanisms to therapeutic opportunities. Nat Rev Mol Cell Biol 22, 75-95, doi: 10.1038/s41580-020-00314-w (2021)). Senescent cell removal or reversing senescence phenotypes ameliorate aging-related disorders (Baker, D. J. et al. Clearance of p16Ink4a-positive senescent cells delays ageing-associated disorders. Nature 479, 232-236, doi: 10.1038/nature10600 (2011)). To elucidate whether GB regulates cellular senescence in aged mice, we compared the expression of senescence signatures in multiple tissues. Real-time PCR revealed that GB administration significantly inhibited the expression of cell cycle checkpoint blockading factors, p16, p19 and p57, in liver, kidney, lung, spleen, heart and muscle, except p16 in spleen and p57 in lung (FIG. 7A). IHC staining revealed that GB administration also reduced the expression of histone γ-H2AX, a DNA damage marker, in multiple tissues (FIG. 7B). In line with GB reducing senescence-associated secretory phenotype, such as IL-6 (FIG. 6), GB administration systematically reduced senescence in aged mice.

Mesenchymal stem cells or mesenchymal stromal cells (MSCs) reside in virtually all mammalian organs, and are responsible for tissue homeostasis and regeneration. Senescence of MSCs with age highly relates to degenerative diseases. Rejuvenation of senescent MSCs has been reported to be associated with improvement of healthspan and amelioration of tissue degeneration. To examine whether GB directly ameliorates senescence in human MSCs, we treated GB to H202-induced senescent MSCs and assessed senescence features (FIG. 7C). GB restored the spindle sharp from flattened and enlarged morphology (FIG. 7D), promoted the proliferation (FIG. 7E) and cellular viability (FIG. 7F, p=0.0026), and reduced expression of senescence markers (FIG. 7G) and senescence-associated β-galactosidase (SA-β-Gal) staining (FIG. 7H, p=0.0007) in senescent MSCs. Accumulation of senescence-associated heterochromatin foci and nucleus size were also reduced after GB treatment (FIG. 71). Our results demonstrated that GB ameliorated oxidative stress-induced senescence in MSCs. Taken together, GB improved healthspan through targeted cellular senescence, at least in part.

### Example 8 GB Administration is Well Tolerated and Provides Health Advantages in Aged Mice

Since aging is accompanied by inexorable impairment of various tissues and vulnerability to diseases, which are associated with inflammation and senescence, we then observed the effects of GB on kidney, heart, spleen and liver in aged mice to determine potential toxicity or benefits. GB administration decreased the compensatory hypertrophy of the kidney and heart but had no effect on liver and spleen size. Through histopathological analysis, we found that GB reduced the aging-related compensatory hypertrophy of glomerulus (p<0.0001) and cardiomyocytes (p=0.0018). GB ameliorated the decrease in white pulp and white pulp/red pulp ratio in aged spleen (p<0.0001 and p=0.0143, respectively). GB administration decreased the number of hepatic microgranulomas (p=0.0031); GB also reduced aging-related hepatic lipid accumulation by 3.8-fold in aged mice (p<0.0001), which is consistent with previous observation in obese model. In addition, reduction of circulating GOT, GPT and UA in aged mice supported the benefits of GB in liver and kidney. Through Masson's trichrome staining, a modest collagen deposition was observed in multiple organs in aged-vehicle mice, which was reduced by GB administration.

We noted that the beneficial effects of GB on multiple organs should be further investigated; however, GB indeed inhibited aging-related pathological changes and reversed the compensatory changes of tissues. We did not find severe acute toxicity of GB in either adult, aged or OVX mice, evidenced by none of the treated mice dying during the two months of GB administration. Short-term pharmacological studies in healthy adult humans revealed that multiple administration of GB was well tolerated and exhibited an acceptable safety profile. Taken altogether, by different aspects of assessments, our results demonstrated that GB administration for 2 months significantly improved healthspan in naturally aged mice.

### Example 9 GB Reconstitutes the Cellular Ecosystem of Aging Muscle

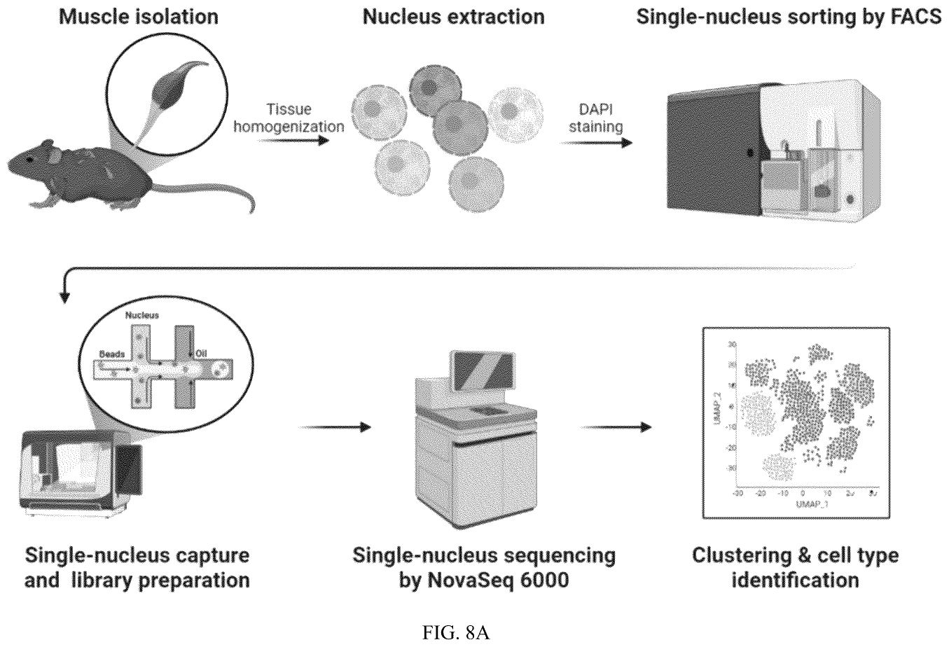

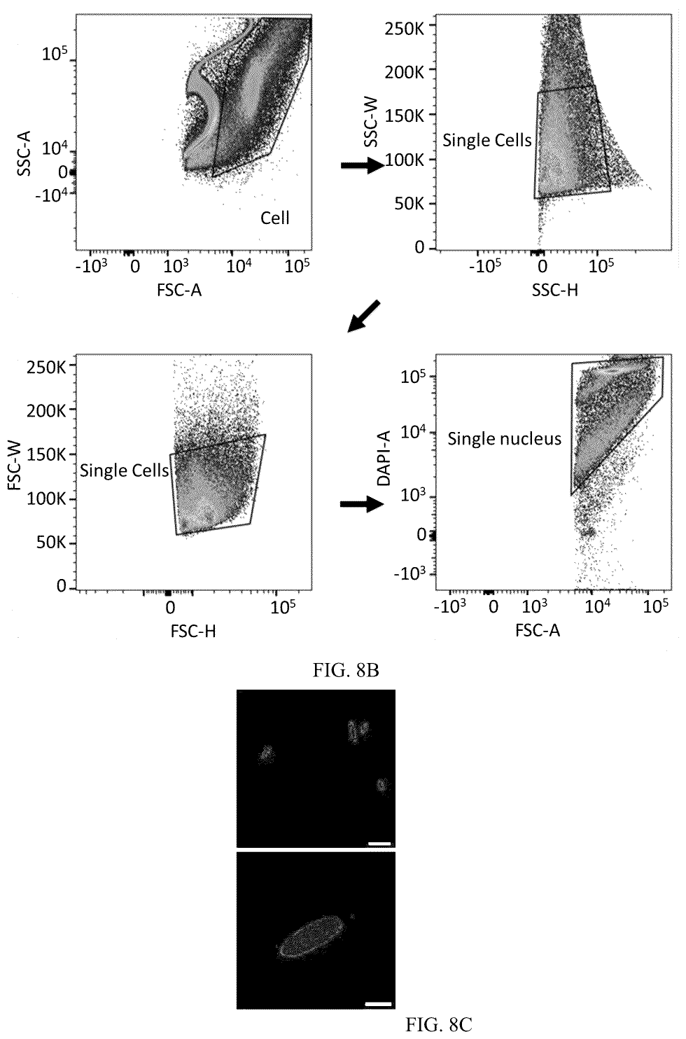

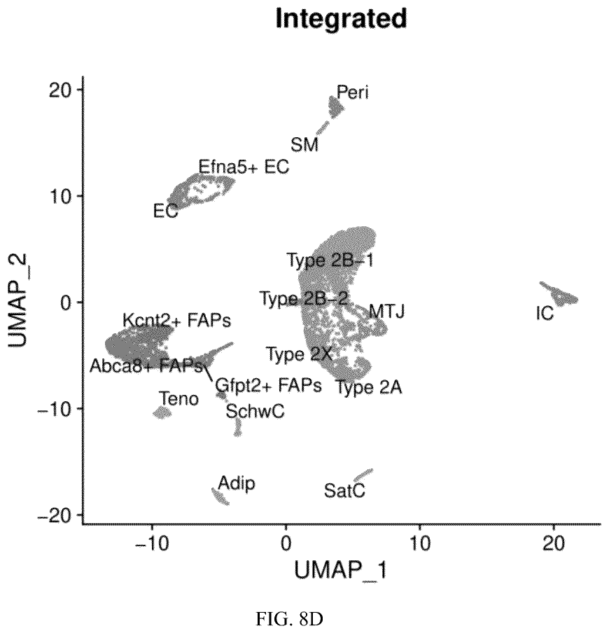

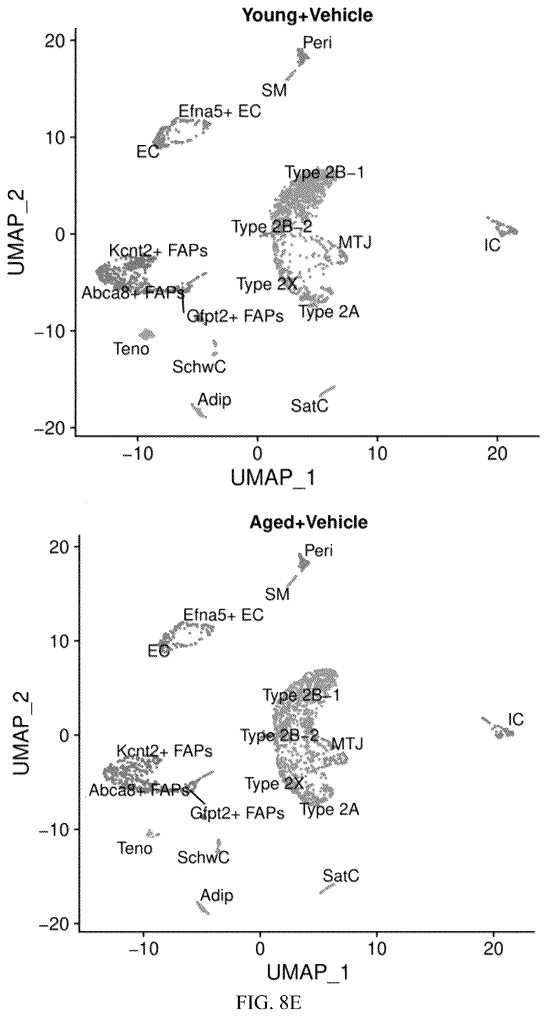

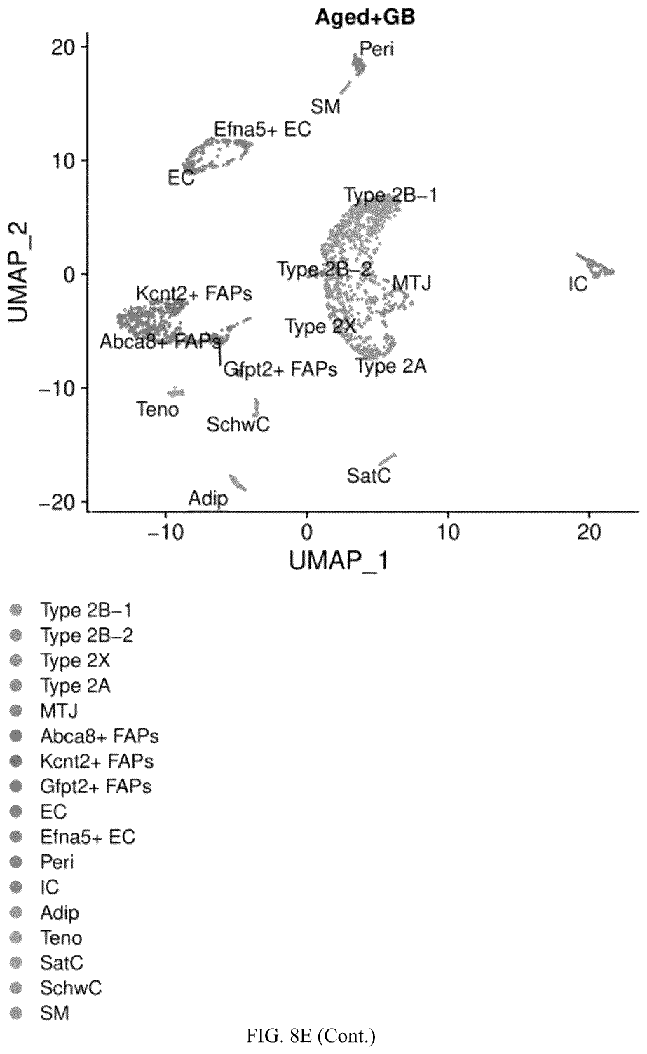

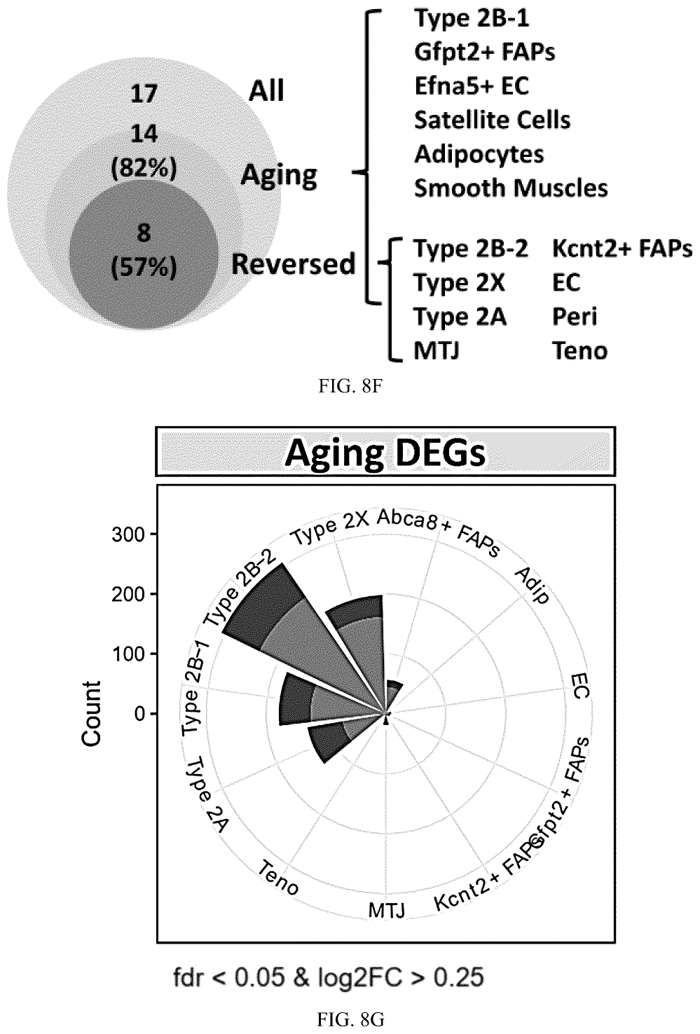

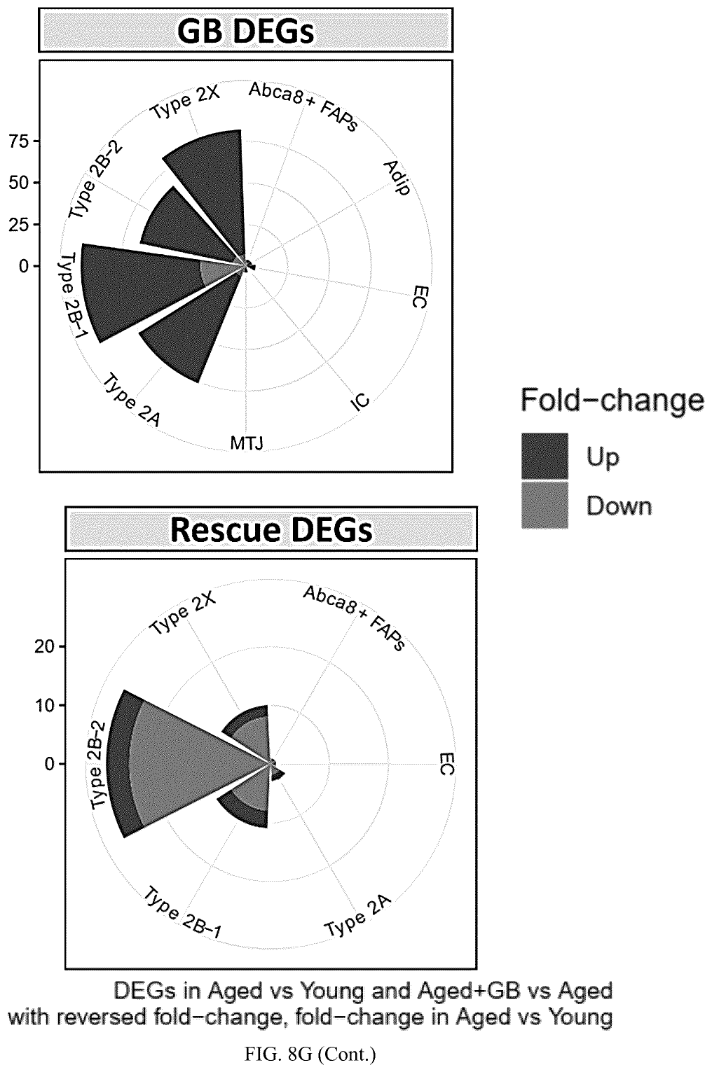

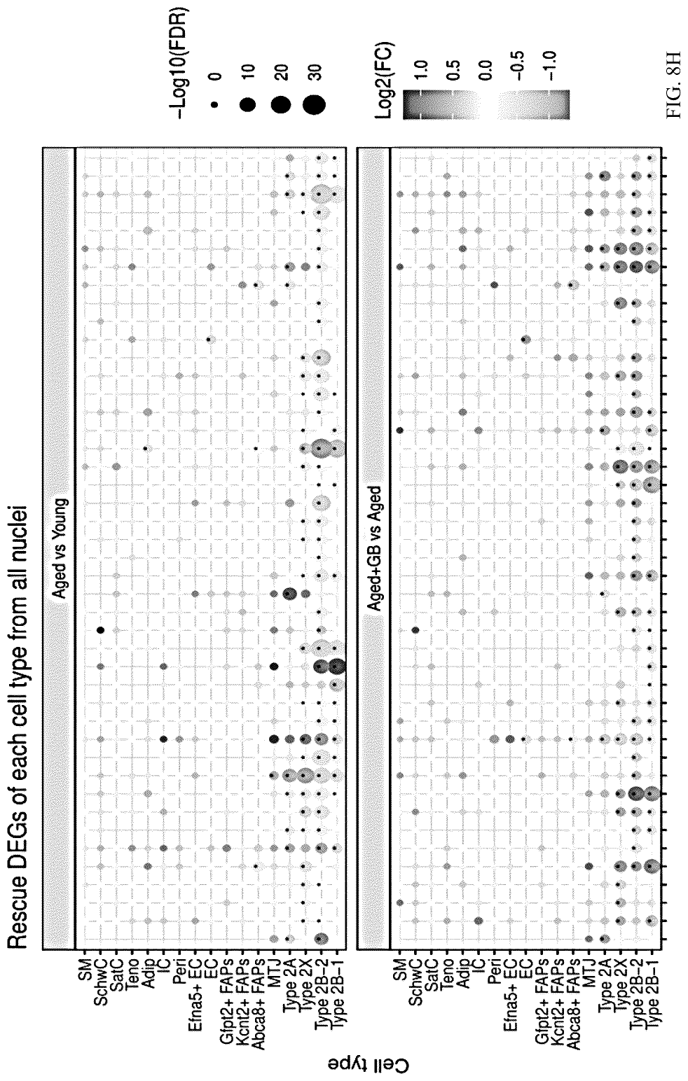

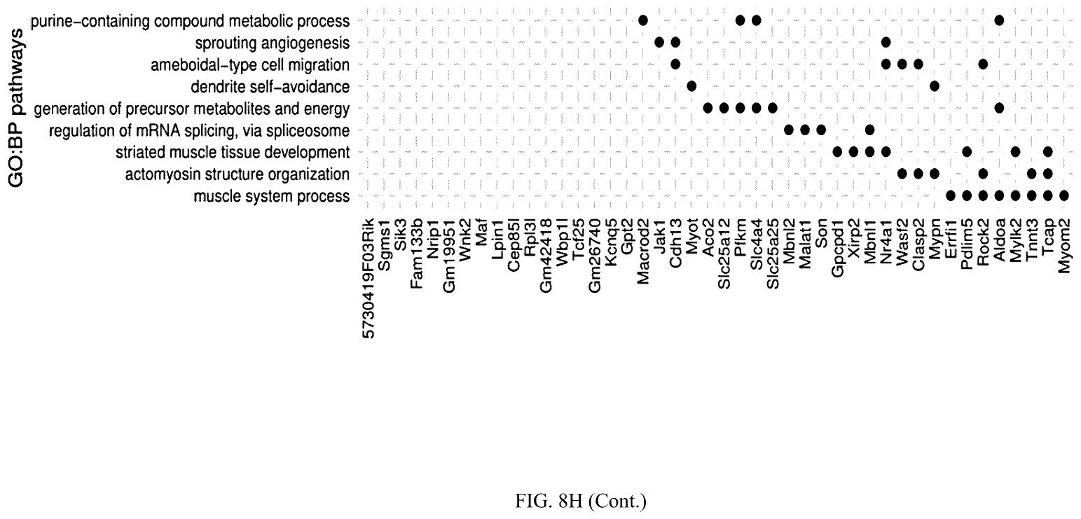

Dynamic changes in cell populations and transcriptomes in skeletal muscles occur during aging, which affect cell-cell communication, metabolic dysregulation, physical declination and frailty (Murgia, M. et al. Single Muscle Fiber Proteomics Reveals Fiber-Type-Specific Features of Human Muscle Cell Rep Aging. 19, 2396-2409, doi: 10.1016/j.celrep.2017.05.054 (2017)). Syncytial myofiber contains hundreds of myonuclei, and each nucleus expresses a specific transcriptome for their nature of work, thus regulating their distinct functions in the nearby location. To gain mechanistic insight into the nature of cellular changes elicited by GB on the skeletal muscle at the single-nucleus level, we isolated the nucleus from the mixed fiber-type GA muscle of young, vehicle-aged, and GB-aged mice by enzymatic digestion and FACS method (FIGS. 8A to 8C) and capitalized on snRNA-seq analysis. To minimize potential bias from individual animals, we pooled two GA from different mice of each group to perform snRNA-seq. A total of 6072 whole single-nucleus transcriptomes were called by the 10x cellranger count pipeline from three individual nuclei. Next, ambient RNA was corrected by SoupX package, cells with more than 5% mitochondrial gene count and less than 250 features were removed, and cell-cycle heterogeneity was regressed out; 5992 nuclei were detected passing quality control and visualized by uniform Manifold Approximation and Projection (UMAP) analysis. Unsupervised clustering identified 17 distinct clusters, which were further annotated based on canonical lineage-specific markers (FIGS. 8D to 8E). Aging altered the proportion of cell types in skeletal muscle by 82% (FIGS. 8F to 8G). In aged muscle, the number and proportion of myonuclei were increased, but the satellite cells (muscle stem cells) were decreased, which is consistent with the pattern observed in aging human muscle tissue88. The proportion of other nuclear types, including those from endothelial cells, pericytes, fibro/adipogenic progenitors (FAPs), and tenocytes, were decreased in vehicle-aged mice. GB administration reversed aging-related changes in nucleus proportion by 57% (8/14) (FIG. 8F). To gain insight into the potential role of GB in muscle resident immune cells, we reclustered the Ptprc⁺ immune cells89. Because of the relatively rarity number of immune cells resident in skeletal muscles for rigorous DEG detection, we identified T-cells, macrophages, and dendritic cells via the expression of representative markers. GB increased the number of Ptprc⁺ immune cells in skeletal muscle, particularly M2 and M2-like proliferating (associated with muscle function) macrophages, which is consistent with the immunomodulatory ability of GB.

We analyzed DEGs to uncover the molecular events related to aging and GB administration in skeletal muscle, DEGs between Aged+Vehicle vs Young+Vehicle, and Aged+GB vs Aged+Vehicle datasets were referred to as “'aging DEGs”' and “'GB DEGs,”' respectively. The aging DEGs rescued by GB were referred to as “'Rescue DEGs”'. Based on the number of DEGs, transcriptomes of myonuclei, in particular those of Type 2B myonuclei, were affected by both aging and GB, and GB administration modestly reversed the aging DEGs (FIGS. 8G to 8H). The rescue DEGs of all nuclei were enriched in metabolic process, angiogenesis, RNA functionality, muscle system progress and muscle development (FIG. 81).

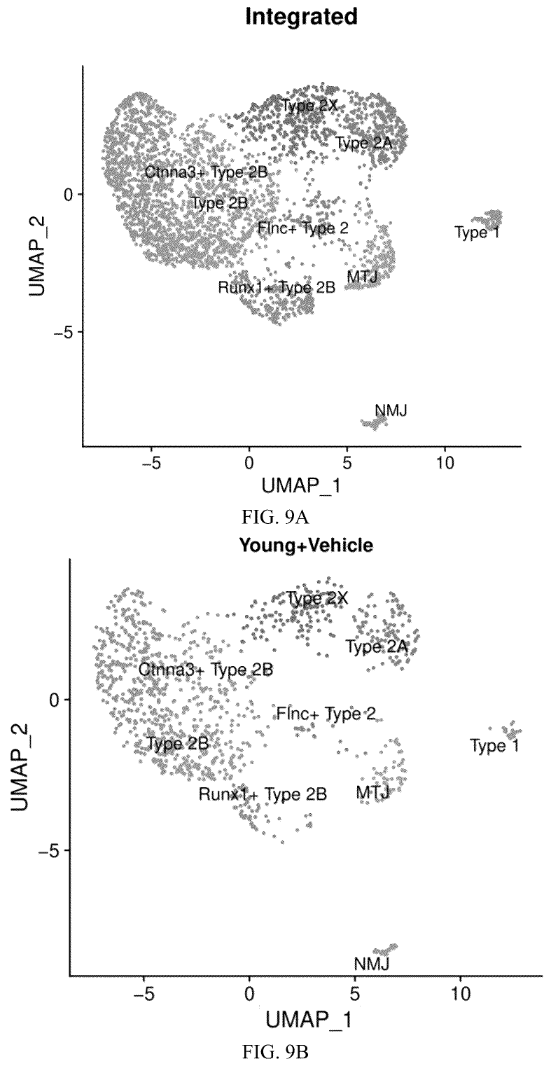

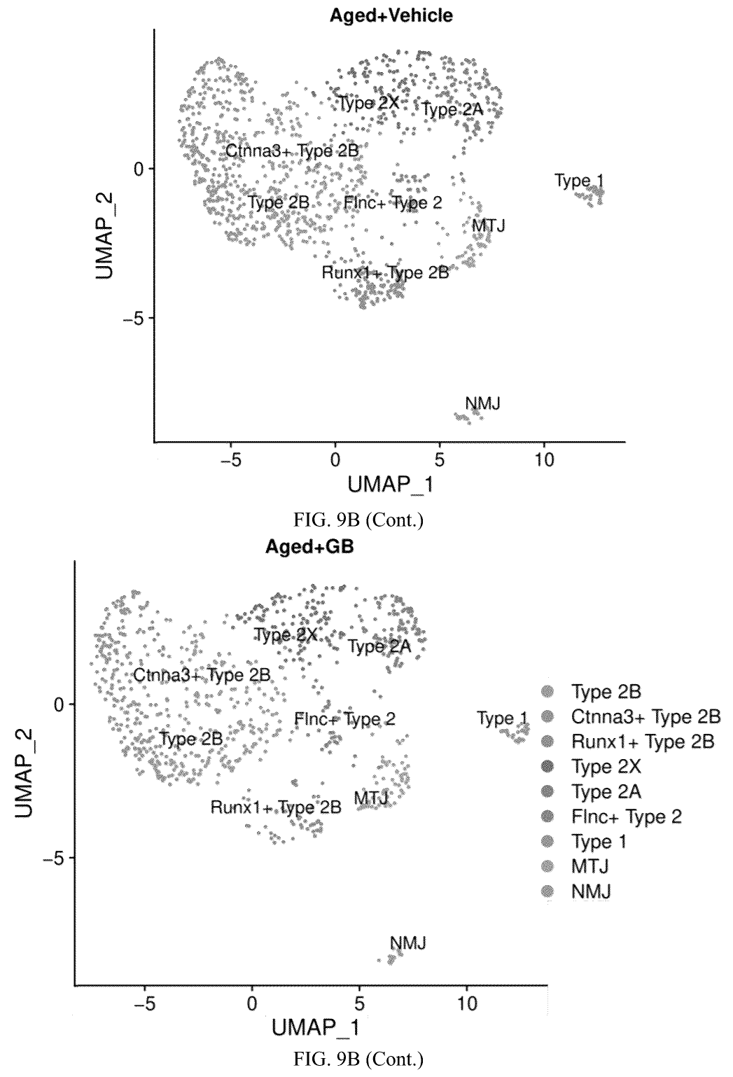

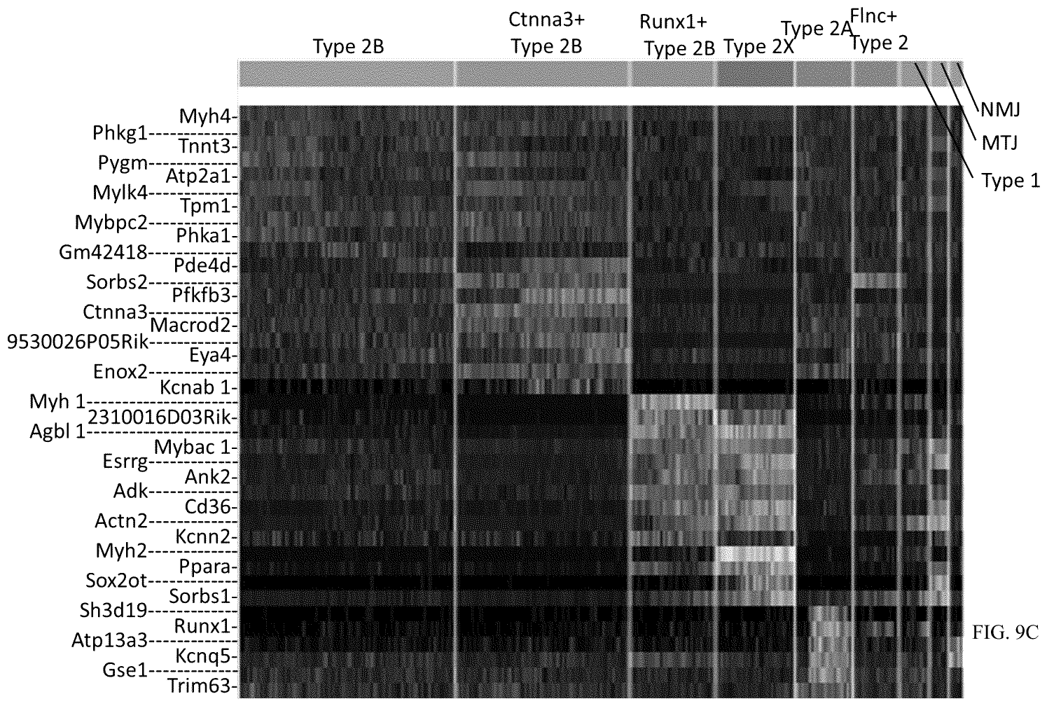

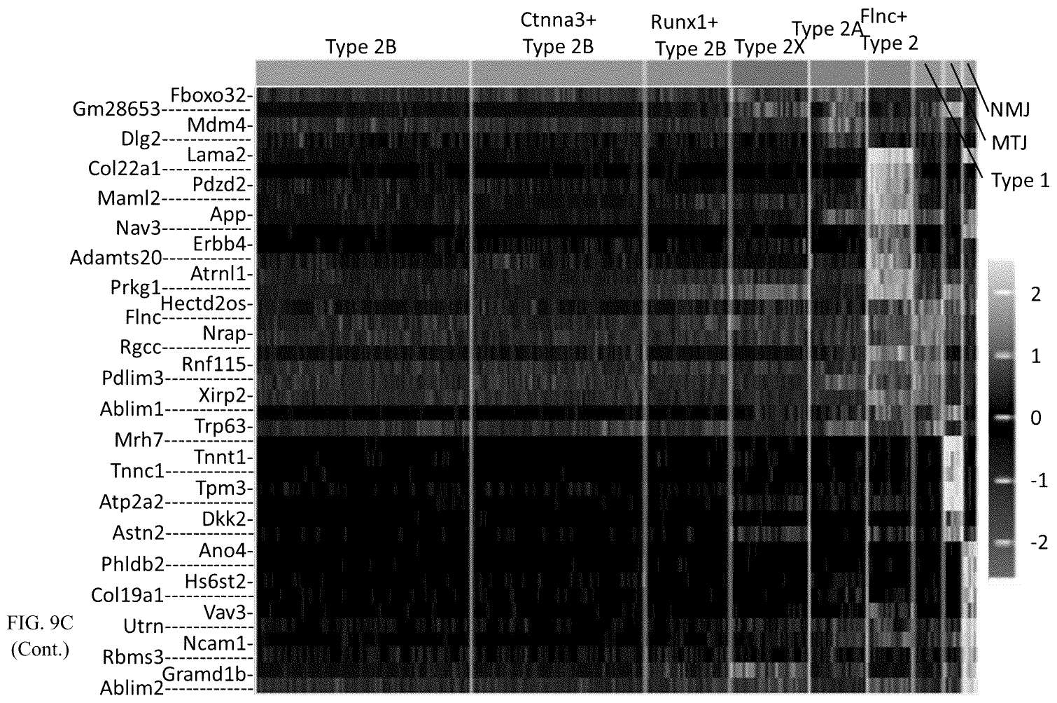

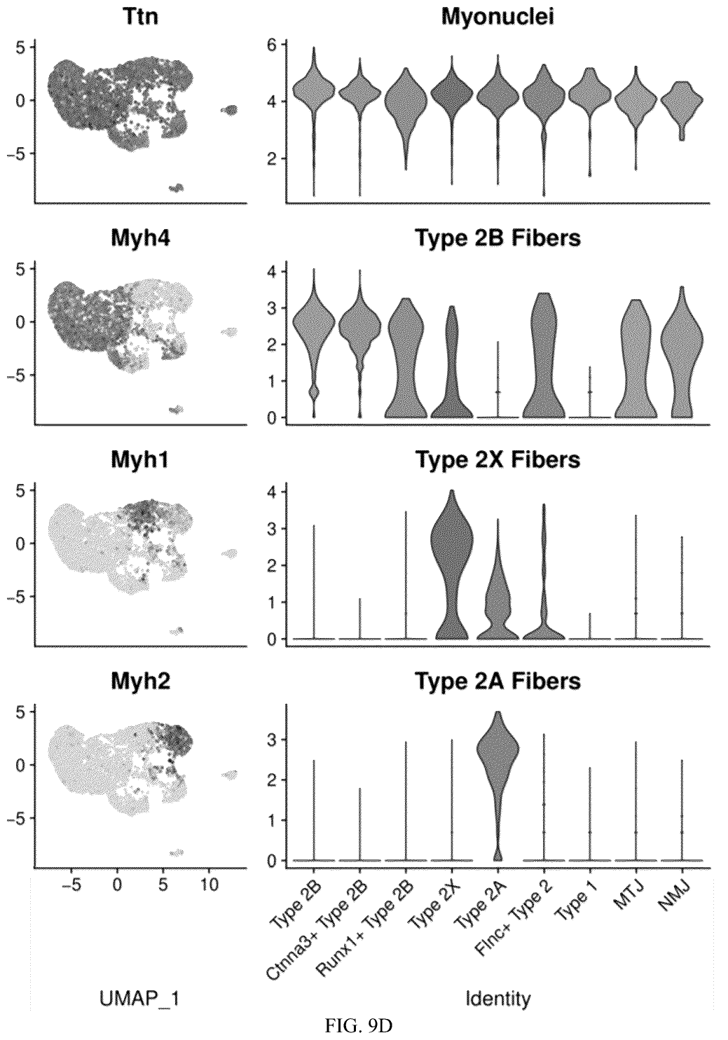

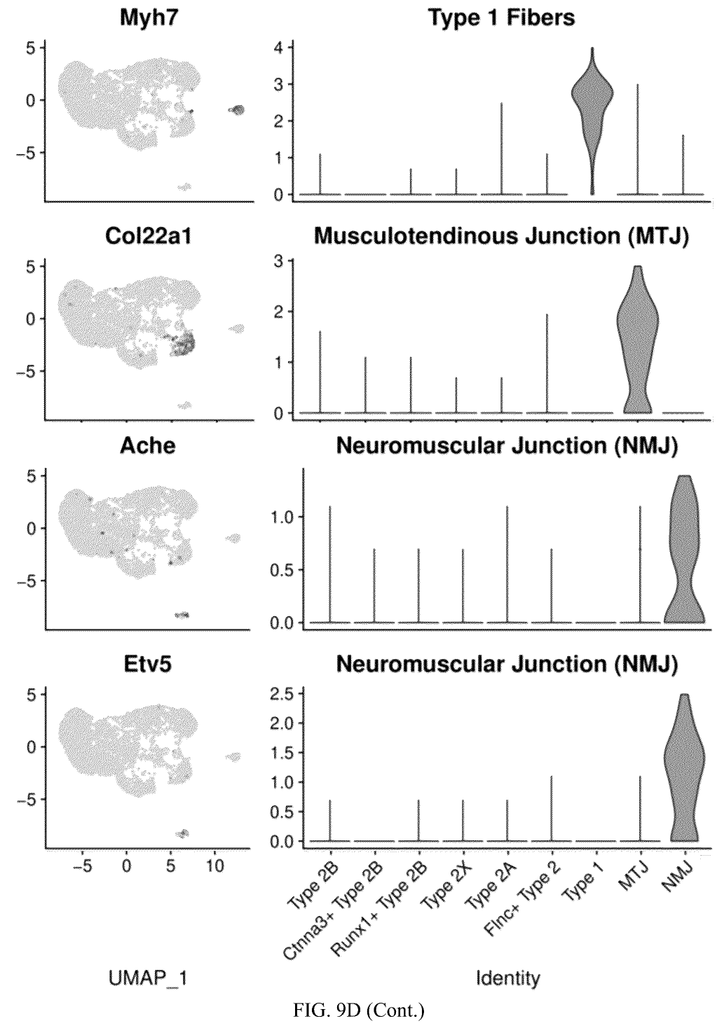

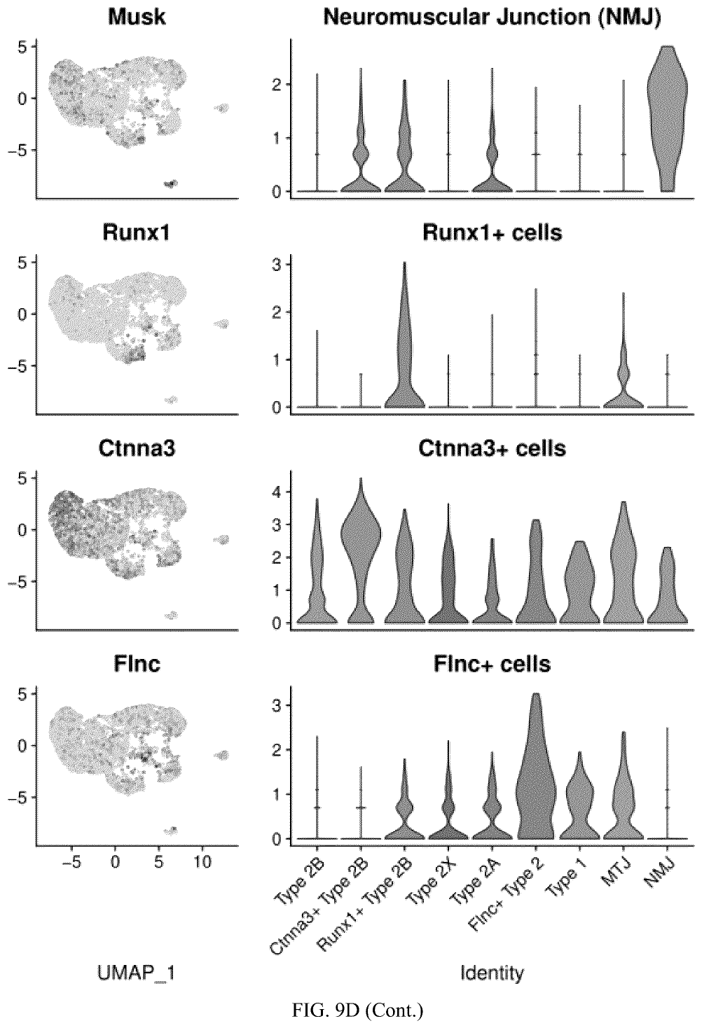

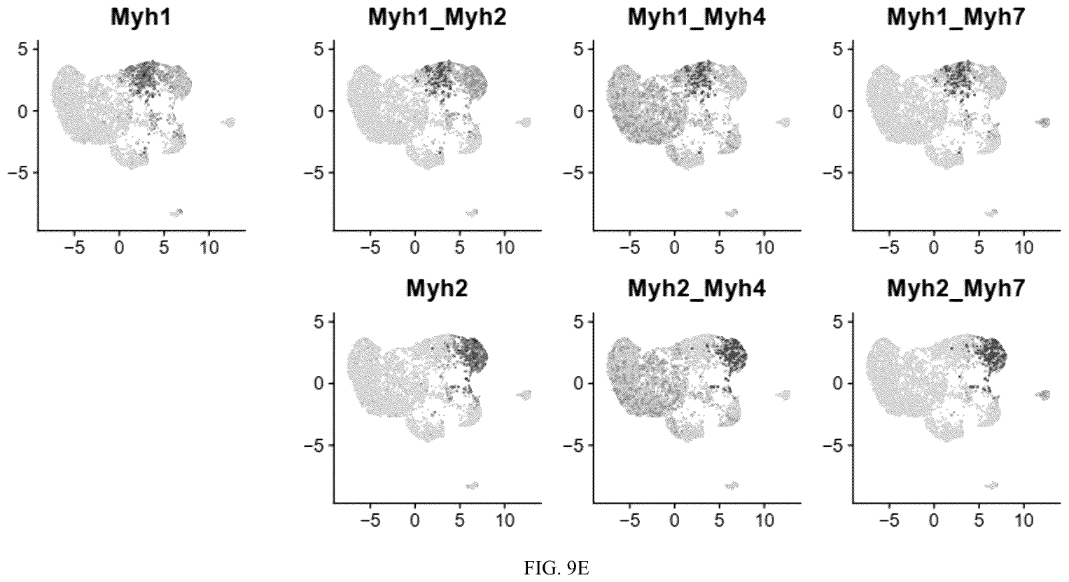

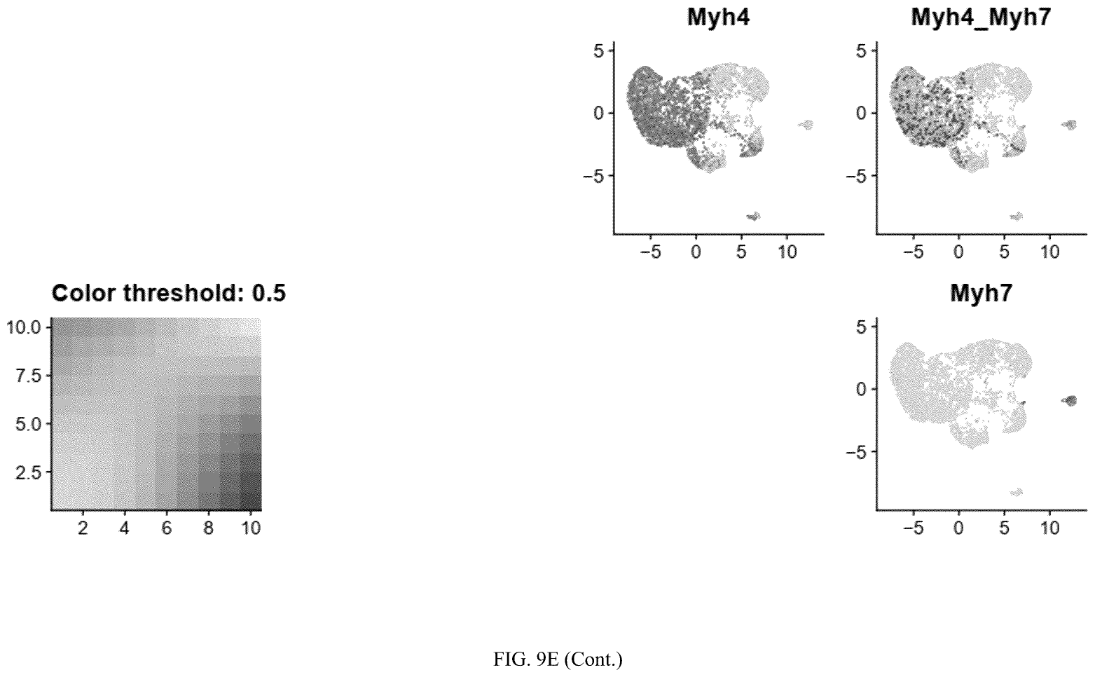

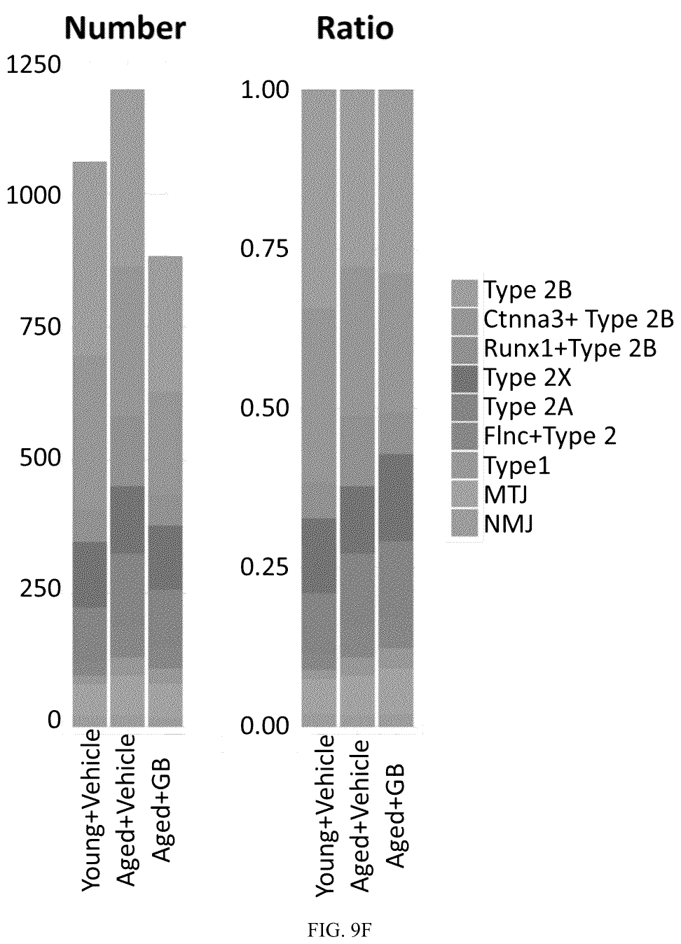

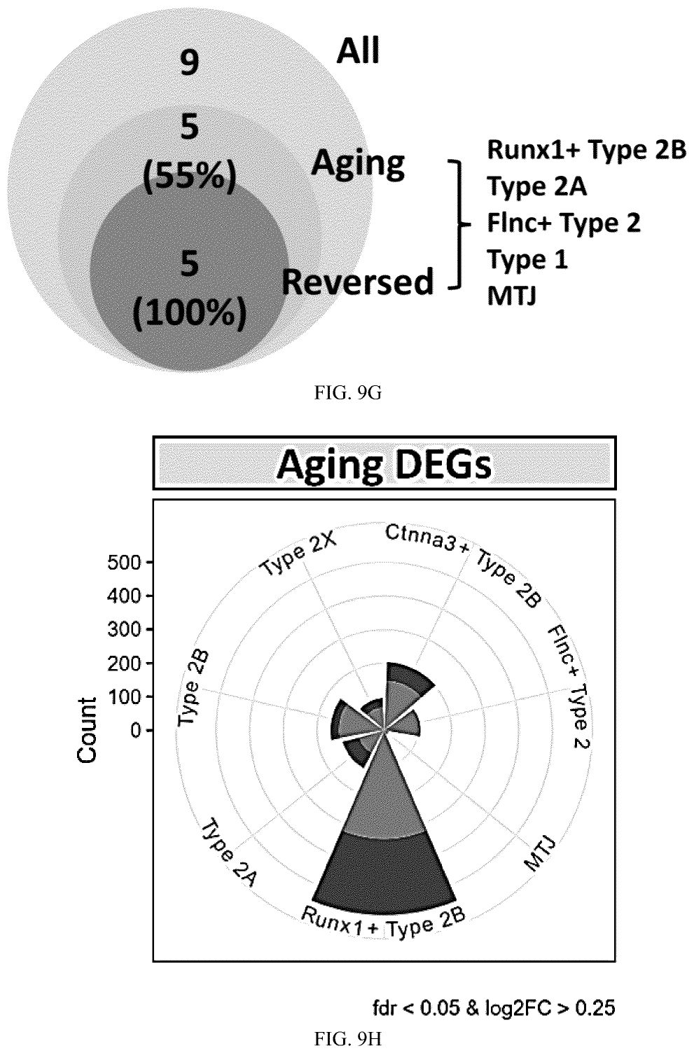

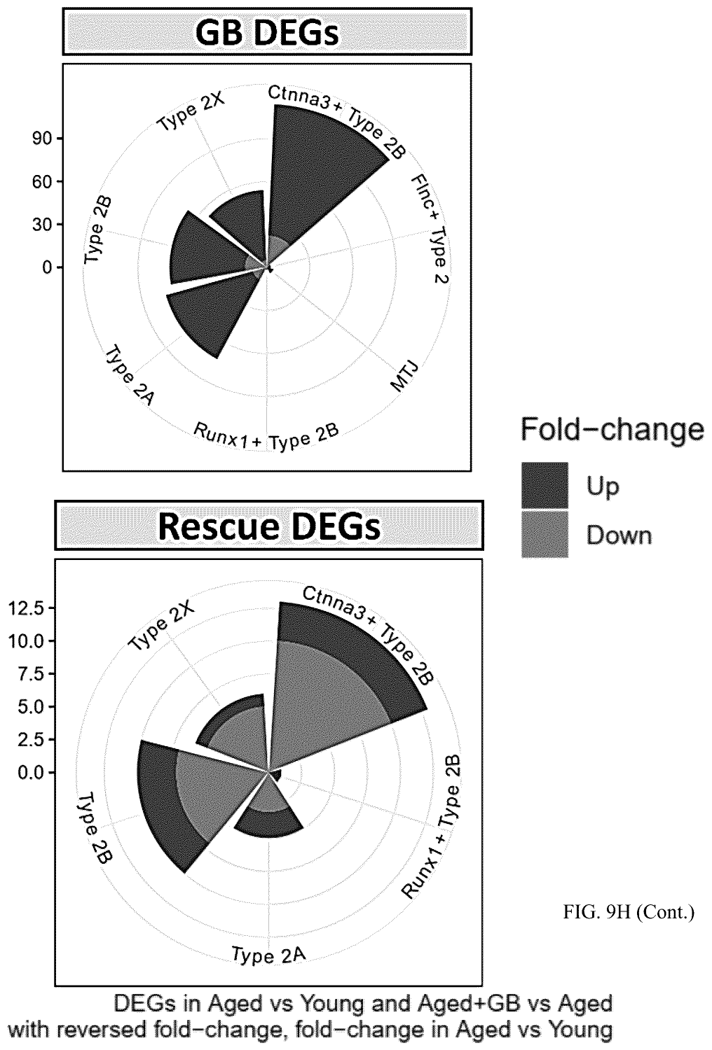

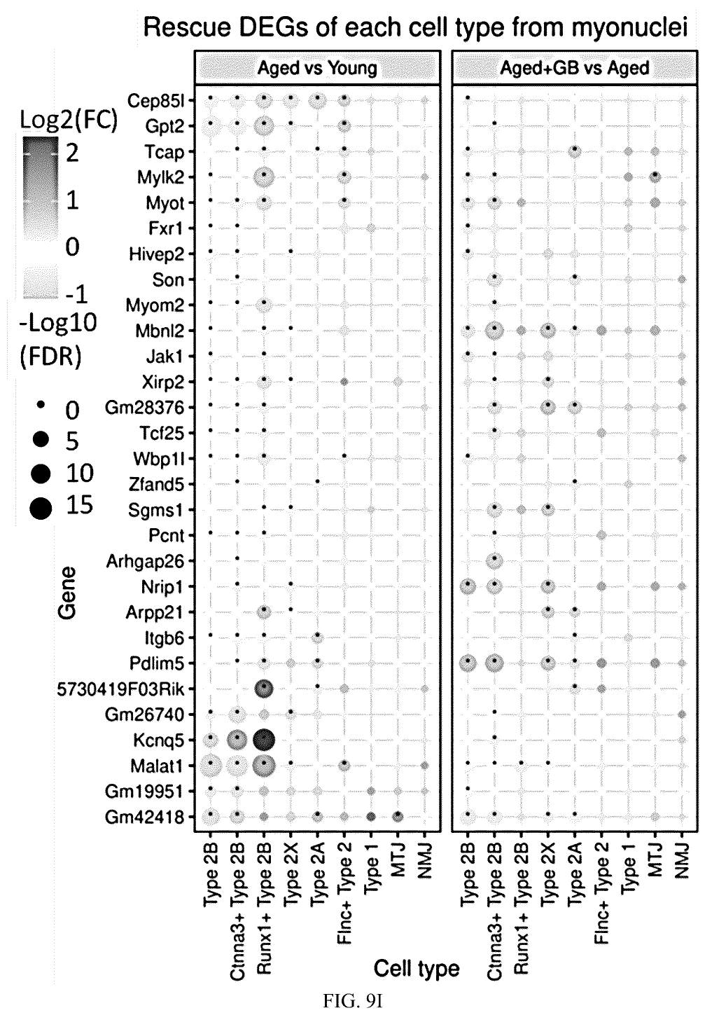

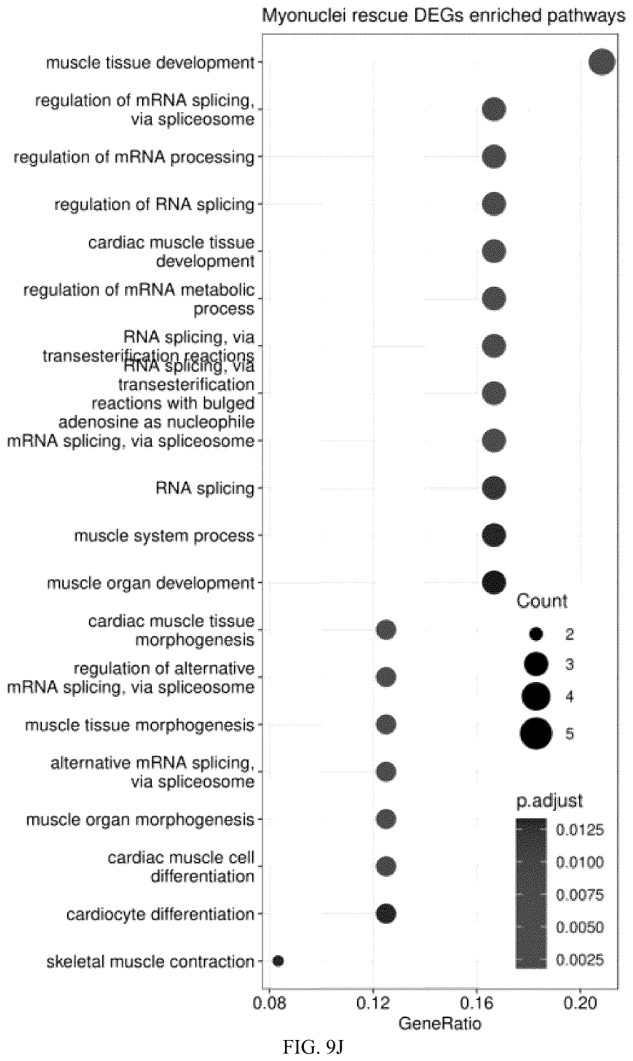

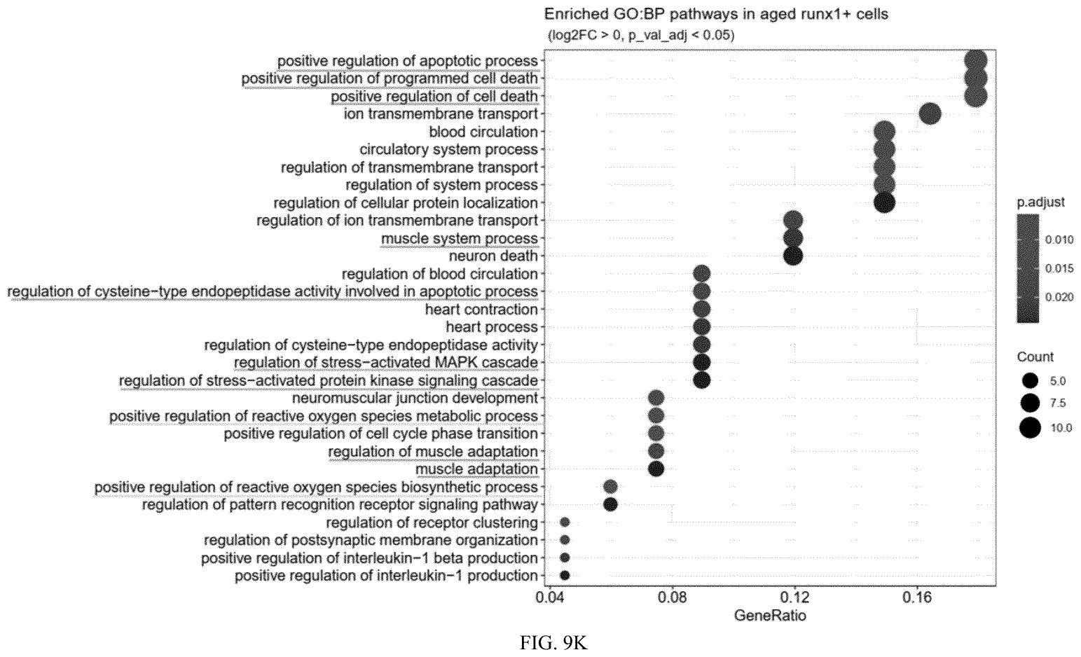

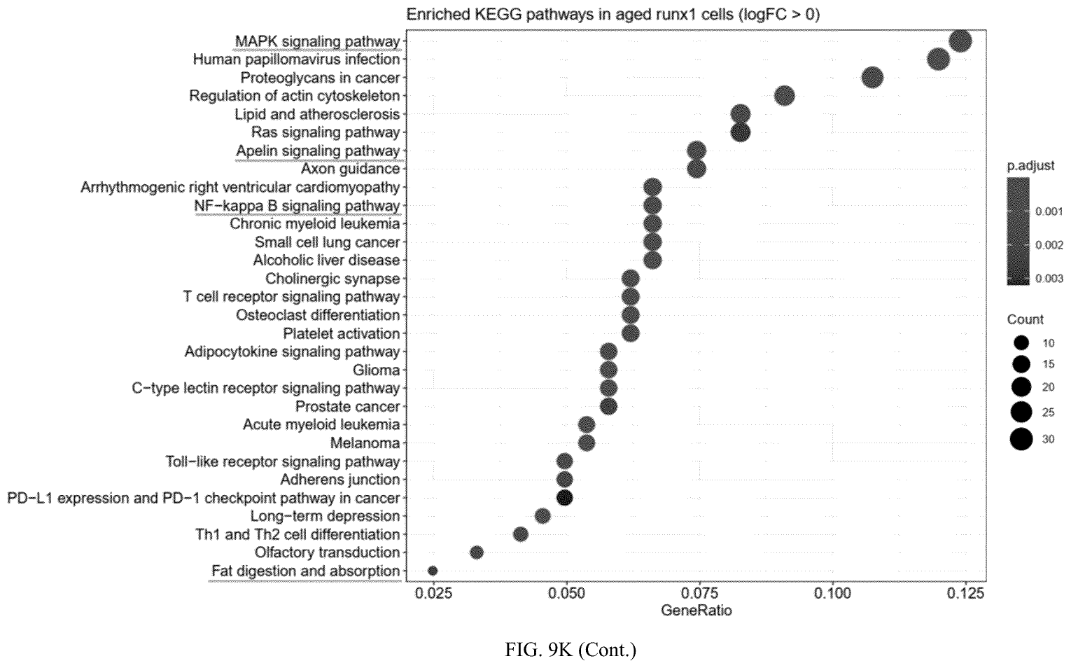

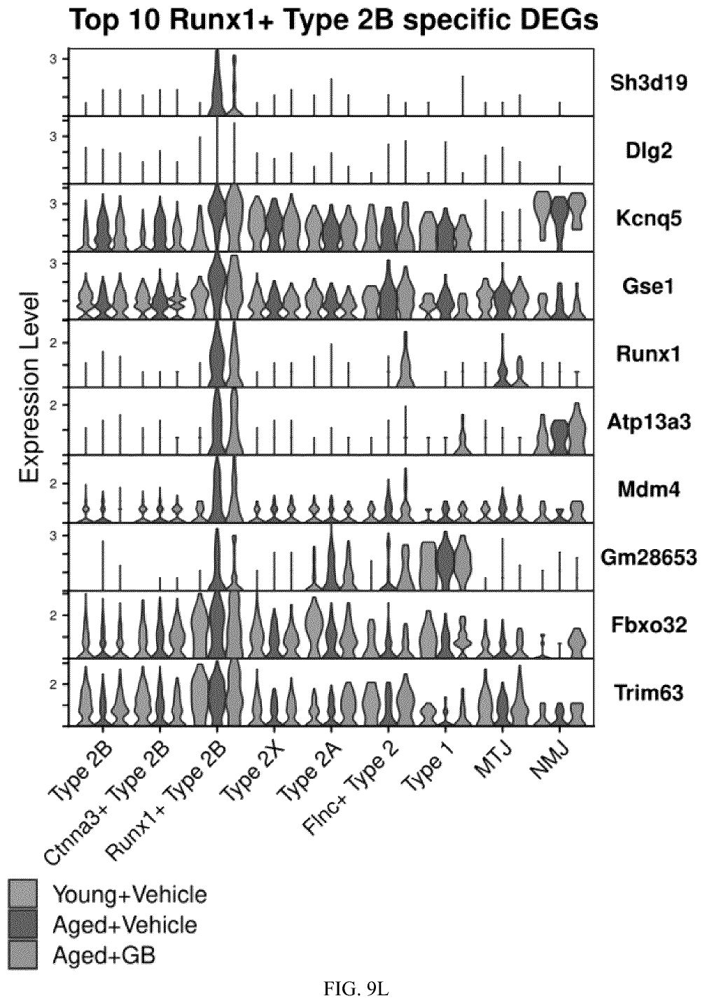

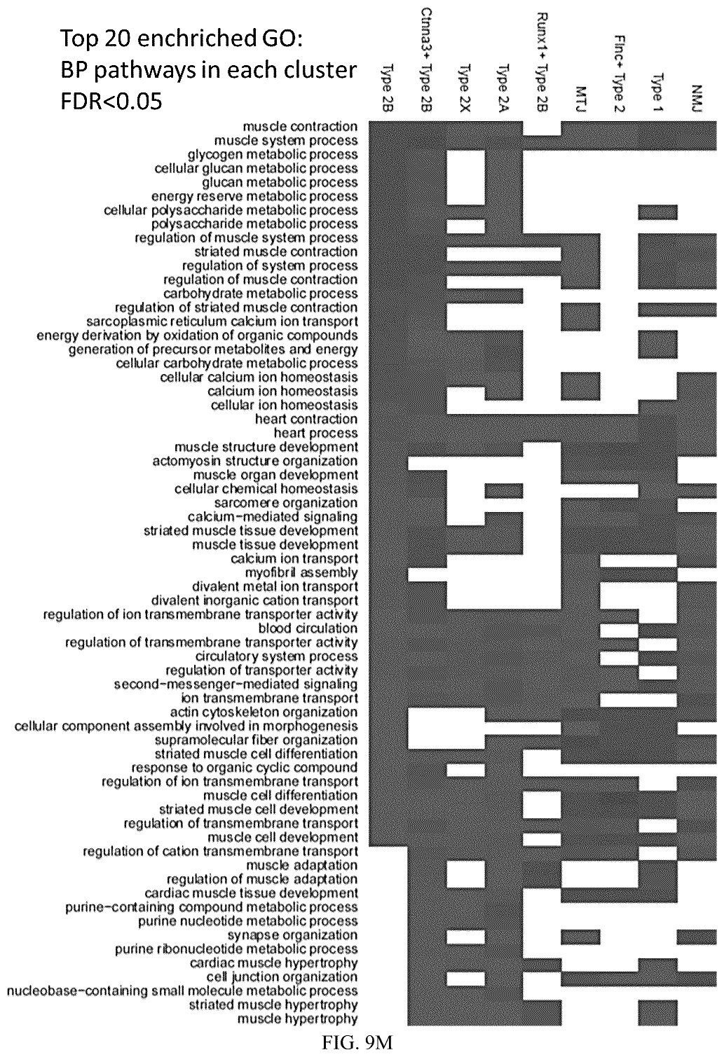

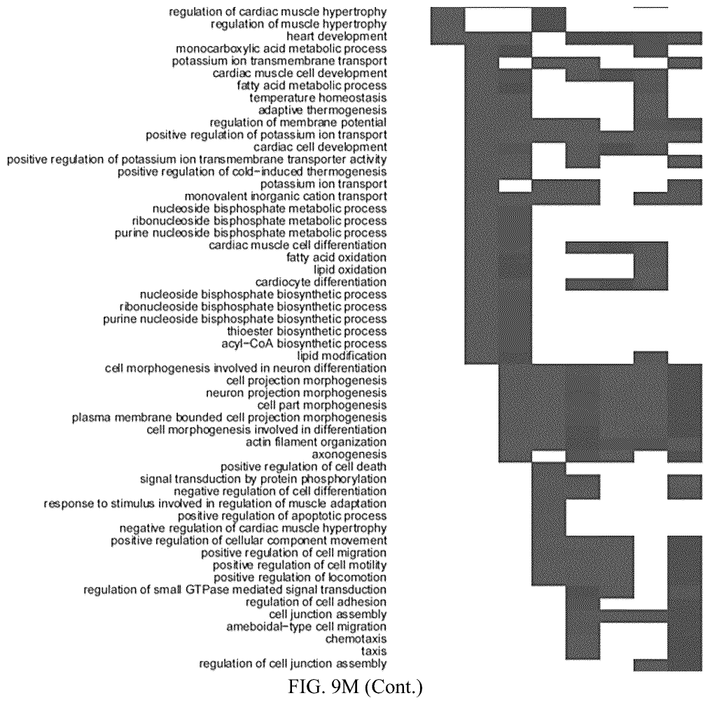

We further analyzed the Ttn+myonuclei, the nucleus type most affected by aging in skeletal muscle. Our results showed that GB partially restored the aging-related transcriptomic changes in Ttn+myonuclei. GSEA analysis from pseudo-bulk DEGs (Young+Vehicle vs Aged+Vehicle and Aged+GB vs Aged+Vehicle) revealed that aging-related myonucleus transcriptomic changes in muscle functions, metabolism, ROS, immune response, inflammation, senescence, and cell death were restored by GB administration, supporting the beneficial effects of GB on physical activity, metabolic health, and senescence. To gain insight into the distinct myonucleus subpopulations, we reclustered Ttn+myonuclei into 9 myonucleus types in the integrated dataset and annotated the clusters based on established myofiber markers (FIGS. 9A to 9D). There seemed to be no obvious hybrid myonuclei, i.e., co-expression of the Myh isoforms within the same myonucleus (FIG. 9E). GB administration reversed aging-related changes in the numbers and proportion of myonuclei, covering all 5 myonucleus subclusters altered by aging (FIGS. 9F to 9G). In myonucleus subclusters, most of the aging DEGs were captured in Runx 1+Type 2B myonucleus, and the largest number of GB DEGs and rescue DEGs were captured in Ctnna3+Type 2B cells (FIGS. 9H to 91). The Rescue DEGs were enriched in muscle development and RNA functionality (FIG. 9J).

Notably, aging most greatly affected the proportion and aging DEGs of Runx 1+Type 2B myonuclei (FIGS. 9F to 9H). Compared with other substances, Runx 1+Type 2B myonuclei showed a unique expression pattern, with Sh3d19, Dlg2, Kcnq5, Gsel, and Runx1 (FIG. 9K). Apoptosis, NF-KB, stress-activated MAPK and ROS cascades were enriched in the Runx1+Type 2b myonuclei (FIG. 9L). By comparing DEGs between young and aged myonuclei, we identified that apoptosis and cell death pathways were uniquely enriched in the aged Runx1+myonuclei (FIG. 9M). Therefore, aged Runx 1+Type 2b myonuclei might tend toward nuclear apoptosis to induce spontaneous muscle degeneration events (the spontaneous central nucleus is a hallmark of degenerative myofibers). These results aligned with the increased proportion of myonuclei/satellite cells and M2-like proliferating macrophages within the atrophic aged skeletal muscle, which respond to muscle injury and microdamage and associate muscle function91. GB administration reduced the expression levels of Runx 1 in myonucleus and the number of aging-elicited Runx 1+Type 2b myonuclei (FIGS. 9F to 9G), which was in line with the observation in the reduction of spontaneous central nucleus in myofibers (FIG. 3M). Given that there was barely any observation of GB DEGs and rescue DEGs in Runx1+Type 2b myonuclei (FIG. 9H), we suggested that GB blunted Runx1 expression to prevent the generation or convert Runx 1+type 2b myonucleus to other myonuclear types.

### Example 10 GB Improves Cell-Cell Communication in Aged Muscle Indicated by Silico Prediction

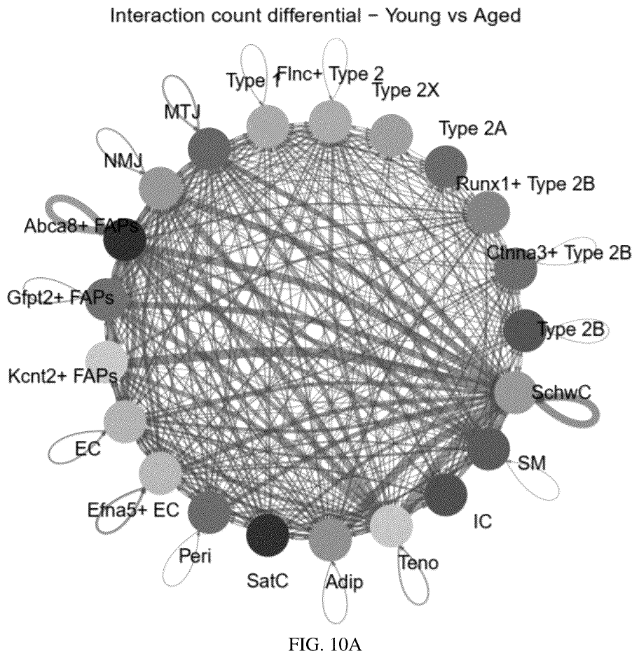

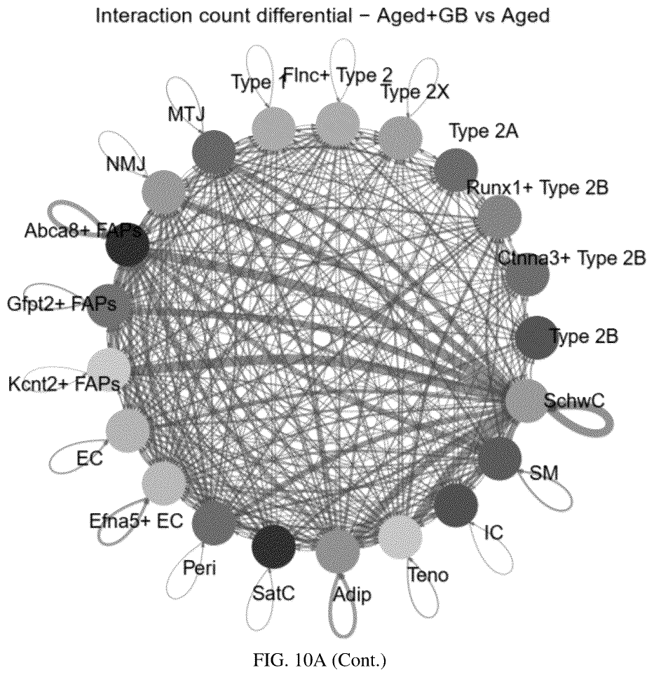

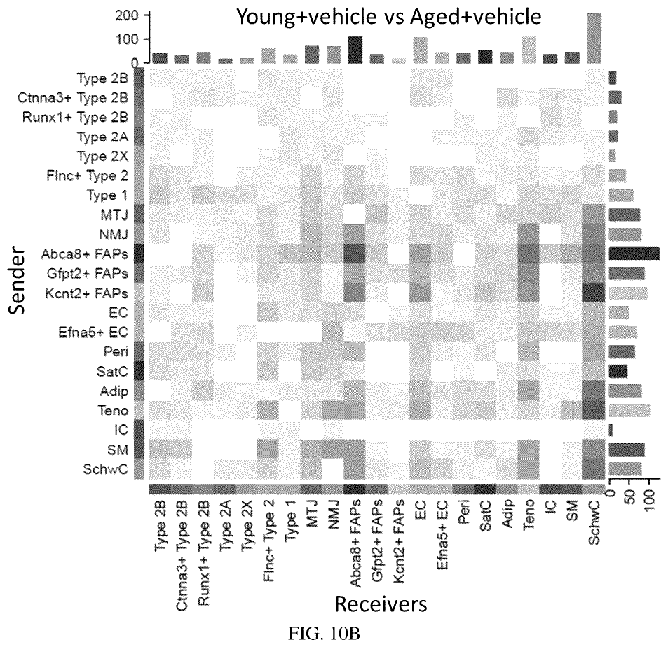

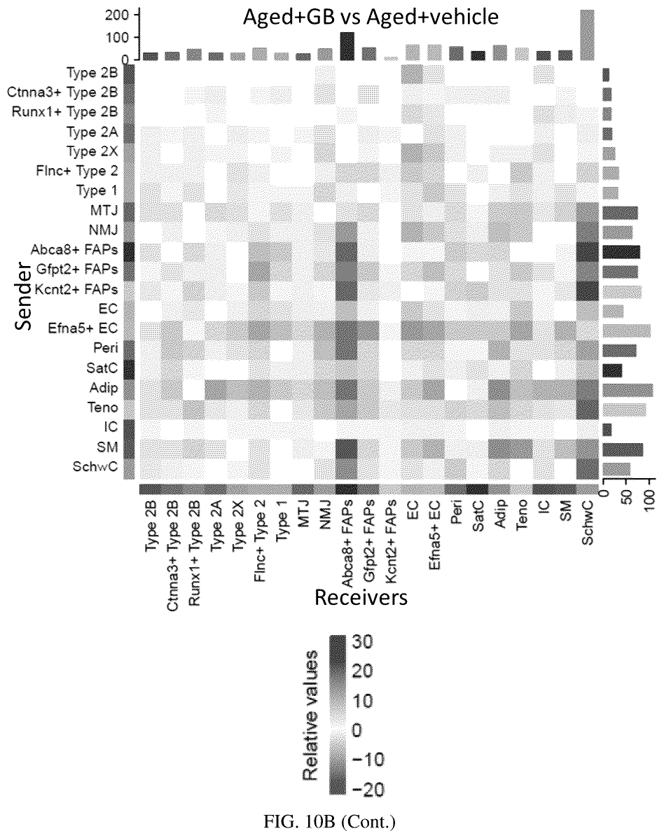

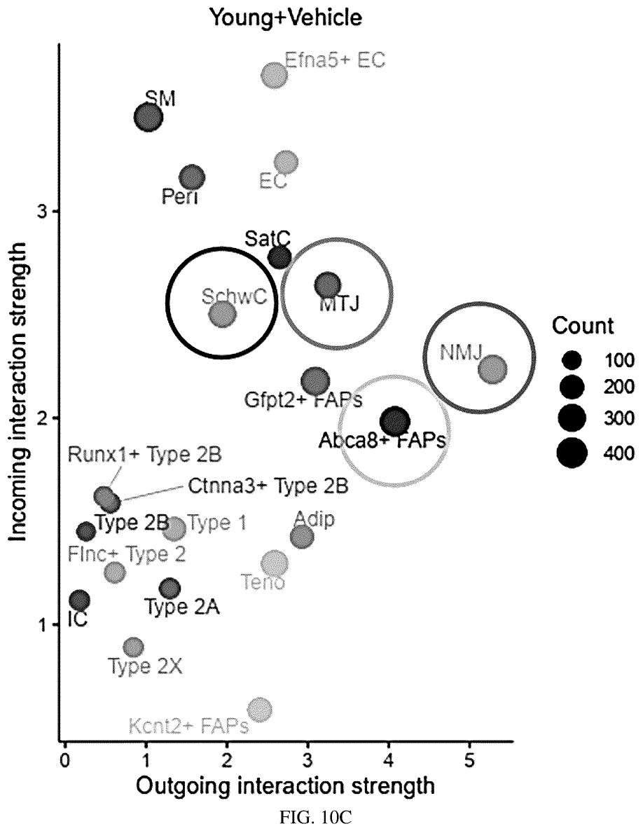

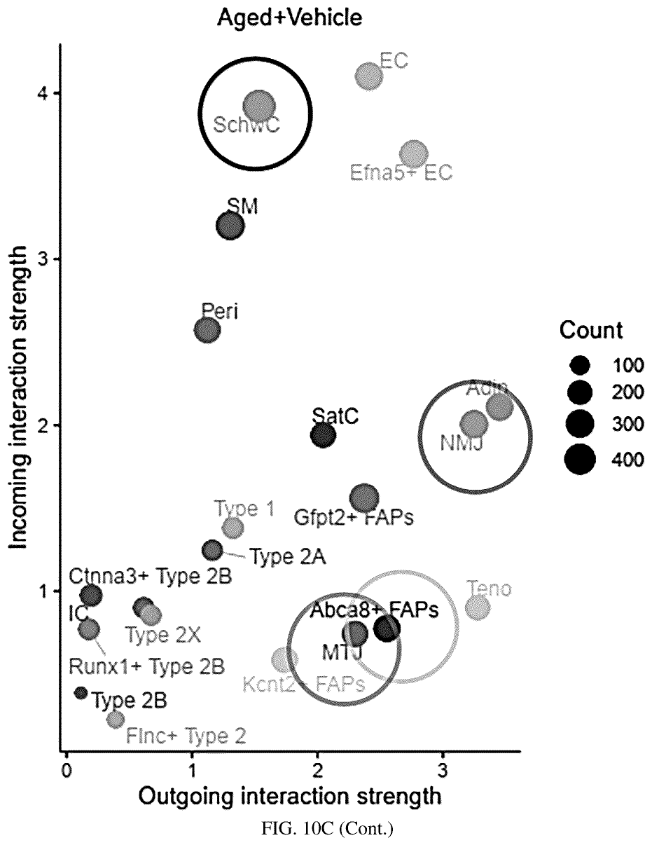

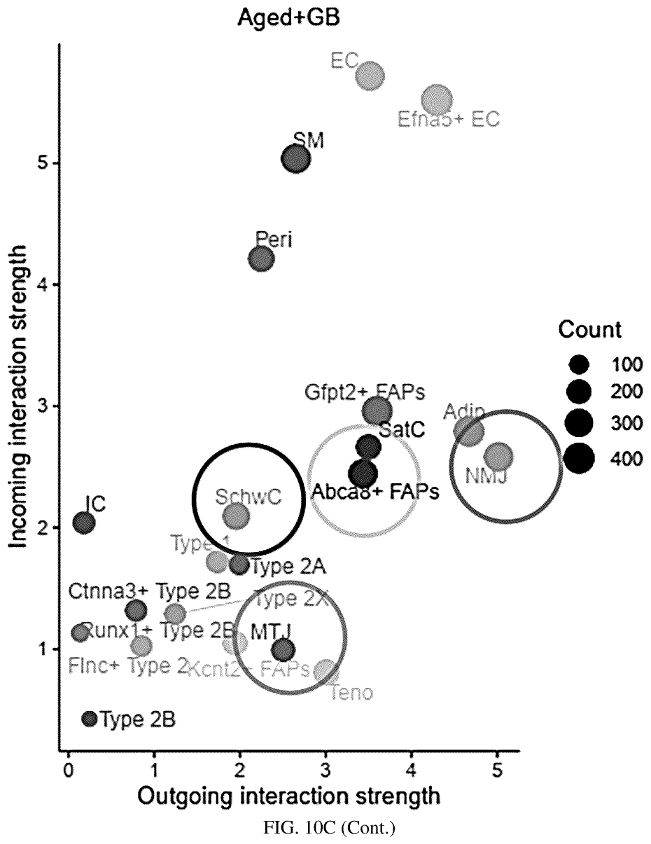

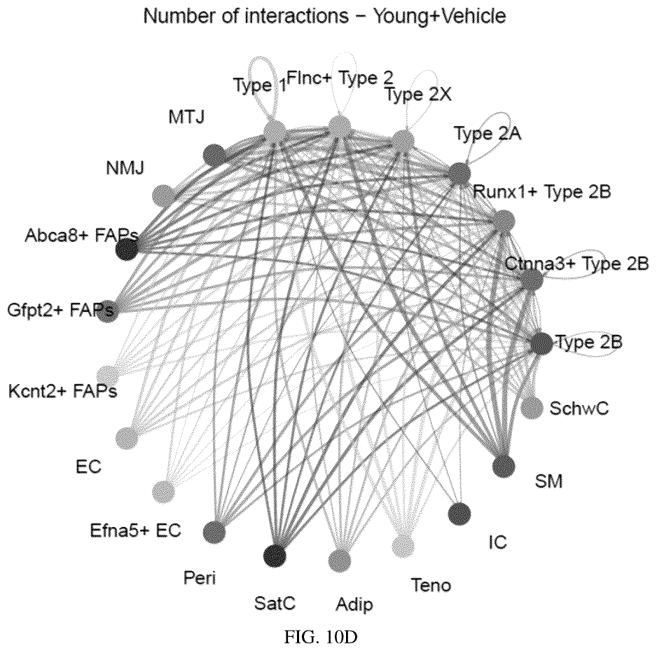

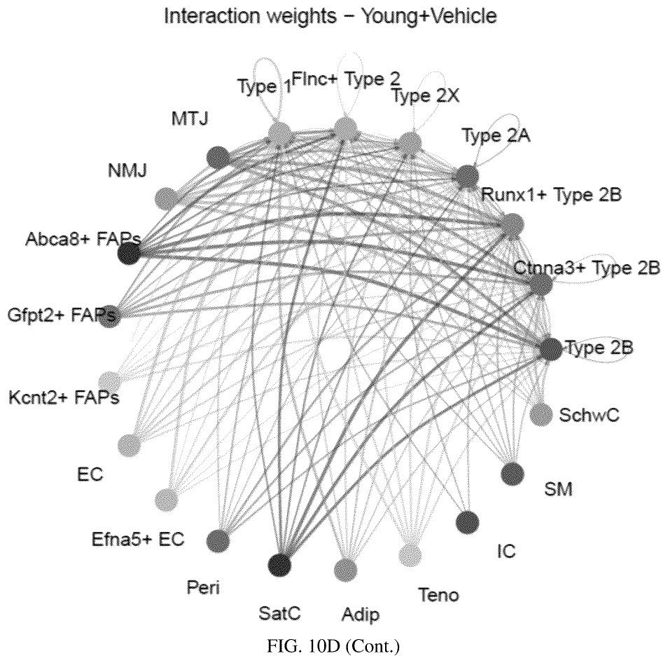

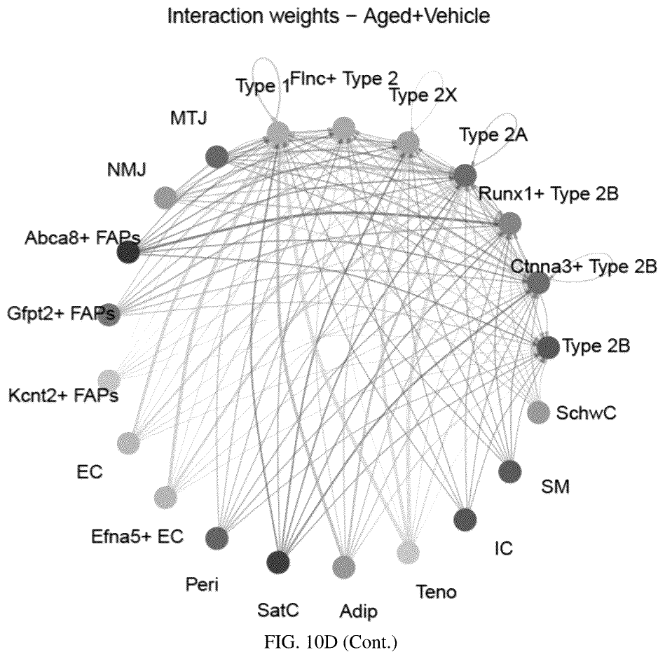

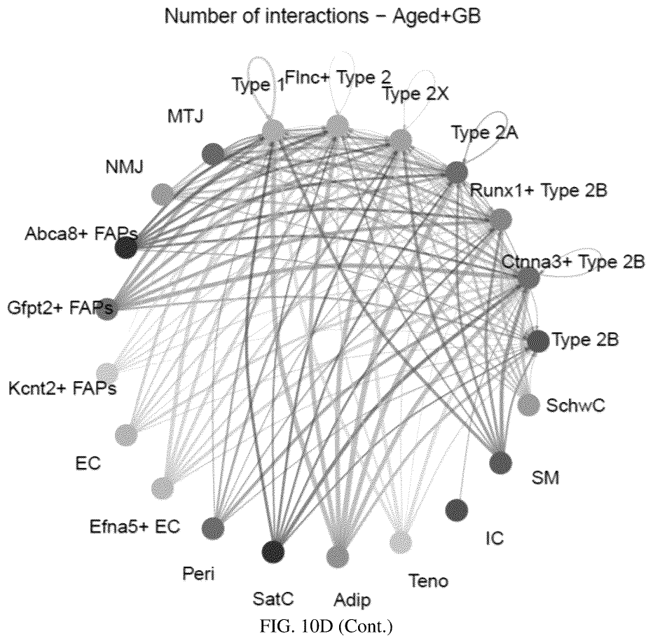

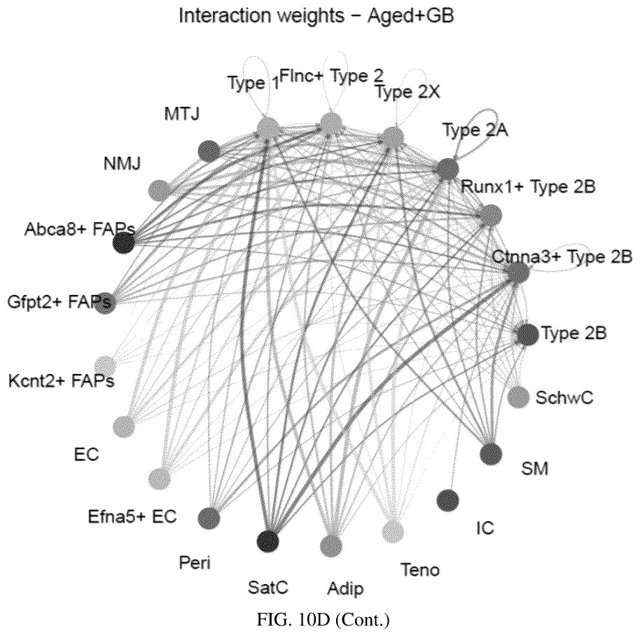

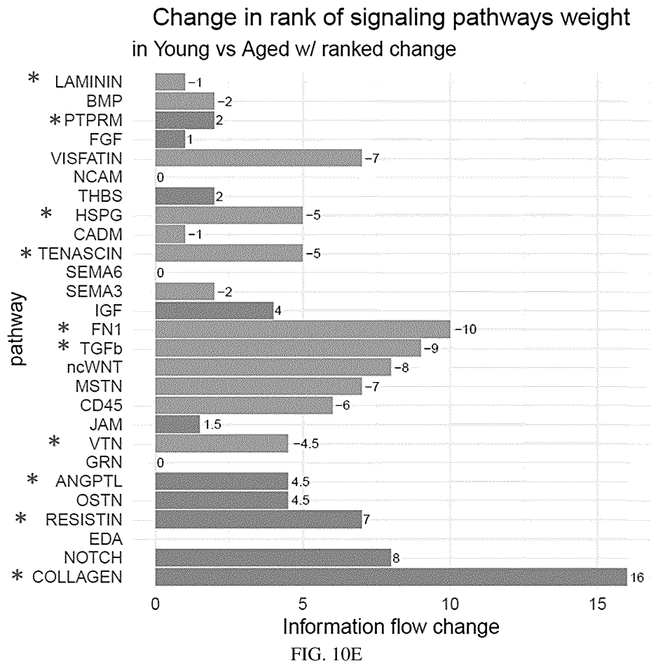

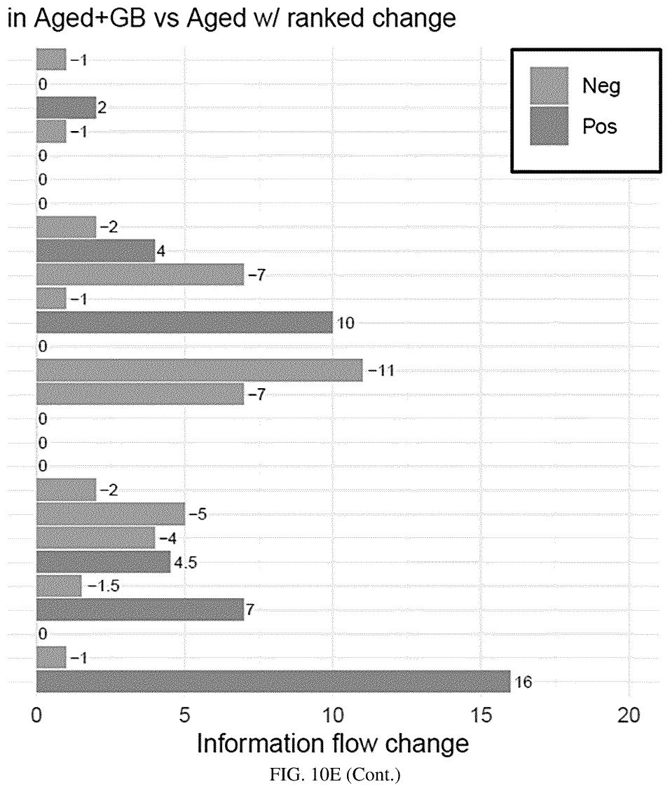

To gather a comprehensive view of cell-cell communication in muscle, we performed ligand-receptor interaction analysis. Ligand-receptor interaction orchestrates an intricate network of cell-cell communication, and abnormal cell-cell communication pattern has been observed in aged muscle. GB administration partially restored the aging-related changes in intercellular cell-cell communication by 57% (20/35) (FIGS. 10A to 10B). For instance, in aged muscle, outgoing signaling from myotendinous junction (MTJ) and neuromuscular junction (NMJ) (FGF signaling) was reduced, while incoming signaling towards Schwann cells was increased (COLLAGEN and LAMININ signaling), and signaling towards Abca8+ FAPs (COLLAGEN and LAMININ signaling) was decreased. Such abnormalities were restored after GB administration. FAPs, neuromuscular junction, adipocytes were the major senders, and endothelial cells, smooth muscle cells, and Schwann cells were major receivers (FIG. 10C). Since muscle cells were not major signal output sources, we looked at the signaling to muscles from other cell types to muscles. The total number of interactions identified between each group was similar, but the inferred strength of interactions was reduced in aged muscle, which was reversed by GB administration (FIG. 10D). Chord diagram showed the GB administration reversed 43% of aging-related cell-to-muscle communications (10/23), including LAMININ, FN1, TGFβ, VTN, HSPG, TENASCIN, COLLAGEN and RESISTIN signaling pathways (FIG. 10E).

Together, GB administration restored muscle nucleus homeostasis in terms of quantity and quality. GB modulated aging-related changes in cell-type composition, nuclear heterogeneity, cell-cell communication, and transcriptomes, particularly inflammation, stress response, apoptosis, and aging hallmarks, which supported our above macroscopic observation in animals.

### Example 11 GB Administration Prolongs Lifespan in Mice

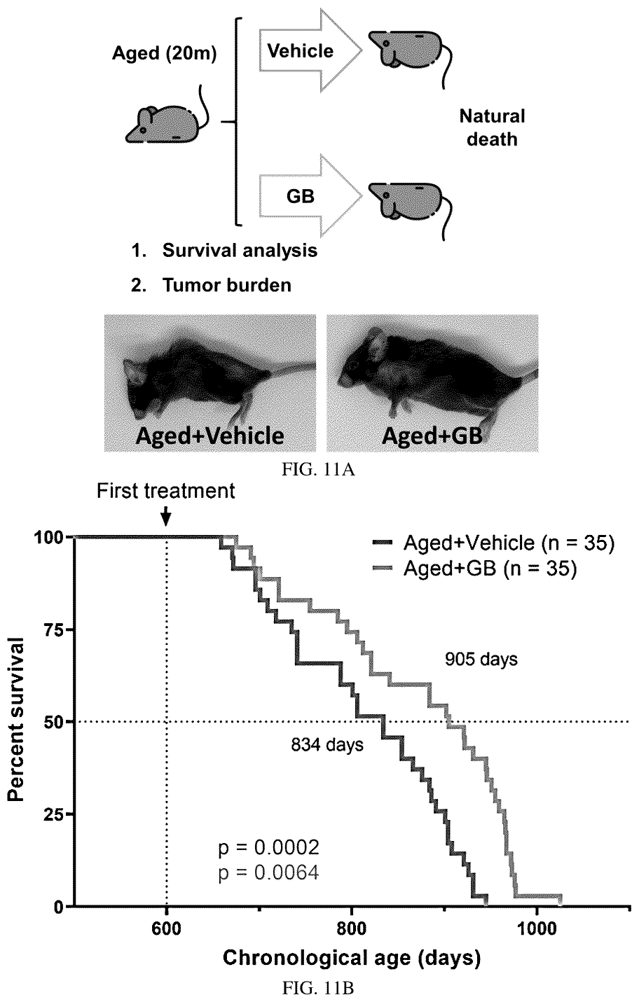

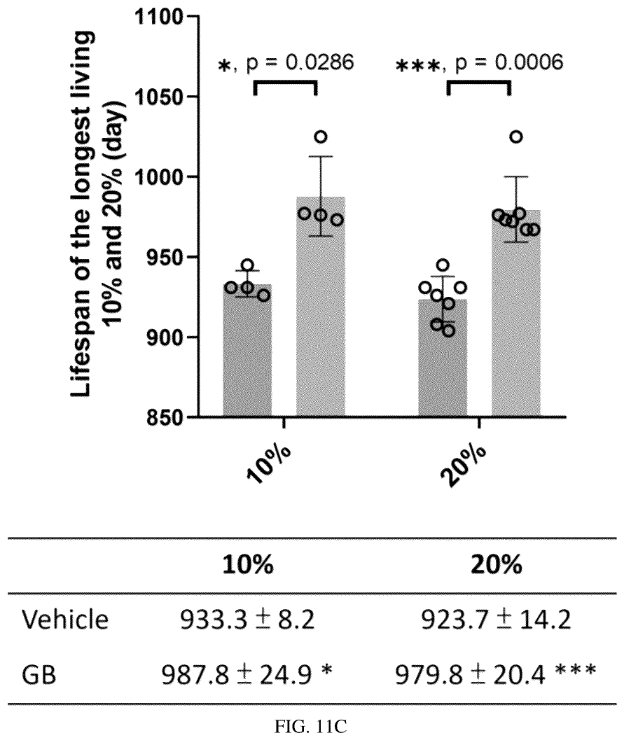

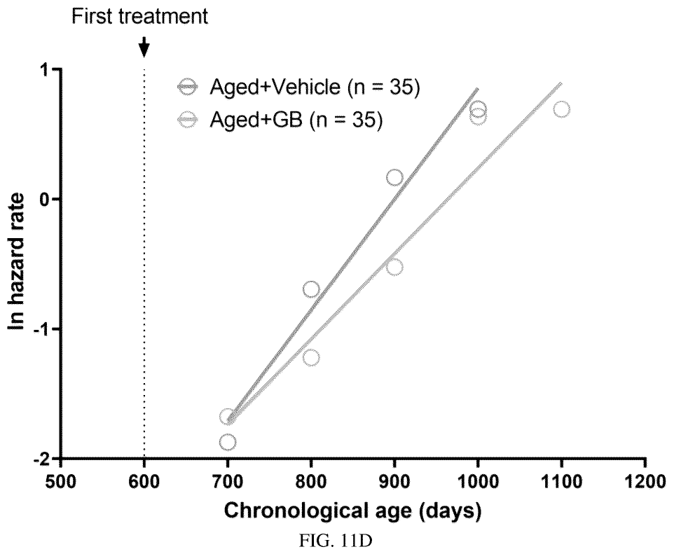

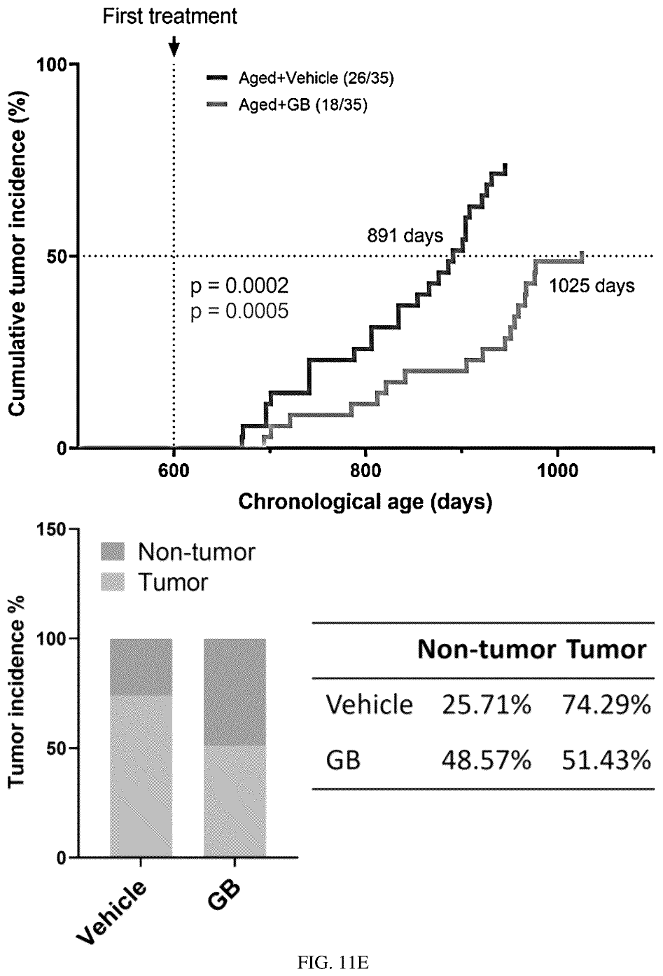

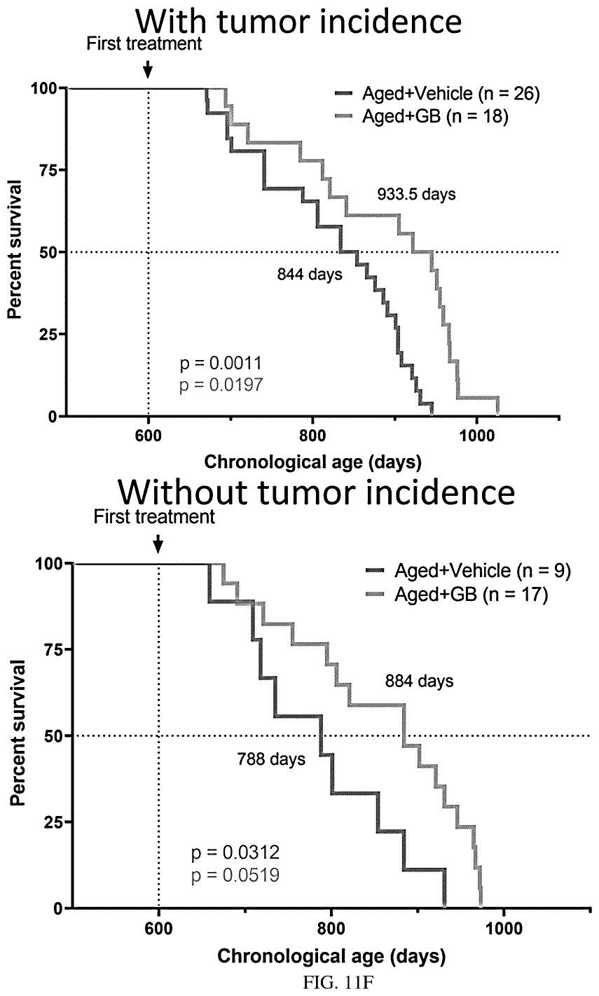

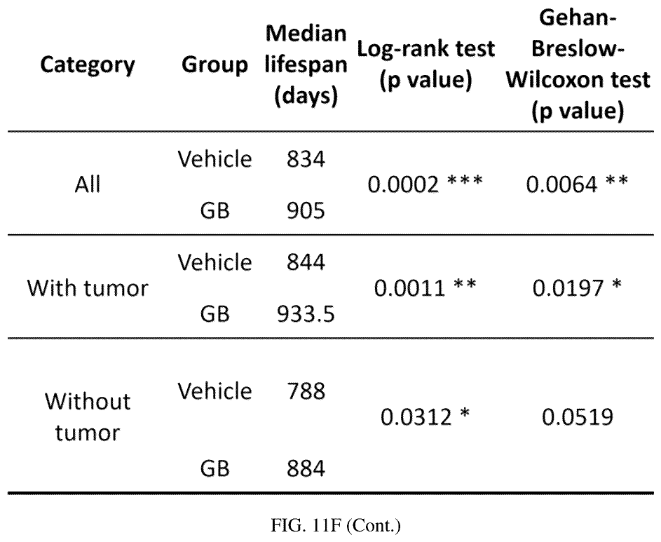

Given the comprehensively geroprotective effects of GB administration on aged mice, we next initiated a longevity cohort of mice at 20-months-old to investigate whether lifelong GB administration starting from old age improved lifespan (FIG. 11A). The mice were not involved in any experiments to maximally reduce potential interference and stress to animals. The mean lifespan of 834 days of the Aged+Vehicle group is consistent with average mean lifespan recorded for C57BL/6J on the JAX Mouse Phenome Database and previous studies (Asadi Shahmirzadi, A. et al. Alpha-Ketoglutarate, an Endogenous Metabolite, Extends Lifespan and Compresses Morbidity in Aging Mice. Cell Metab 32, 447-456 e446, doi: 10.1016/j.cmet.2020.08.004 (2020)). From the inception of GB administration, median lifespan was significantly extended by 30% (p=0.0002 by log-rank test and 0.0064 by Gehan-Breslow-Wilcoxon test), and the hazard ratio was reduced (0.3526 by log-rank test or 0.4577 by Mantel-Haenszel) (FIG. 11B). GB administration significantly extended the mean maximal lifespans of the 10% and 20% longest-lived mice by around 55 days (FIG. 11C, p=0.0286 and 0.0006, respectively), and delayed aging-associated mortality (FIG. 11D). Given that GB has been reported to possess anti-tumor activities, we evaluated the effects of GB administration on spontaneous tumors with aging. Our results showed that less macroscopic tumors were detected by necropsy in the GB-aged mice compared with vehicle group and the cumulative cancer incidences were 50% by age of 891 days and 1025 days in vehicle and GB groups, respectively (FIG. 11E, p=0.0002 by log-rank test and 0.005 by Gehan-Breslow-Wilcoxon test). In addition, median lifespan extensions of GB-aged mice were observed in both tumor and non-tumor cohorts (FIG. 11F, p=0.0011 and 0.03 by log-rank test, respectively). Therefore, the beneficial effects of GB on healthspan and lifespan were attributed to the improvement of multiple organs, not merely the anti-tumor effect of GB.

1. A method for inhibiting and/or reversing aging in a subject, comprising administering an effective amount of ginkgolide B (GB) or a derivative thereof to the subject.

2. The method of claim 1, wherein the method is for reversing aging in the subject.

3. The method of claim 1, wherein the method is further for extending healthspan in pathological and steady-state conditions.

4. The method of claim 1 wherein the method is further for extending lifespan.

5. The method claim 1, wherein the method is further for one of more of the following:
enhancing skeletal muscle mass, enhancing grip strength, enhancing circulating osteocalcin, enlarging the cross-section area (CSA) of the tibialis anterior and/or soleus muscles, increasing skeletal muscle-to-body ratio and/or myofiber CSA, reversing the infiltration of adipose-like tissues by aging, increasing the total protein content in aged skeletal muscle, downregulating the expression level of Fbxo32 or Trim63, reversing aging-related muscle wasting, enhancing force, reversing the aging-related changes in fiber-type switching, reducing the aging-related increase in intramuscular lipid infiltration, collagen deposition or the number of central myonuclei in aged myofibers, enlarging the deep femoral artery or capillary density, reducing body weight progressively, decreasing in whole-body fat mass, increasing lean mass, reversing the aging-related alteration in serum triglyceride or total cholesterol levels, improving glucose tolerance, restoring the expression of Glut4 and Pkm in aged skeletal muscle, enhancing neuromuscular and/or enhancing physical performance in the subject.

6. The method of claim 1, wherein the method is further for reducing frailty score, systemic inflammation, inflammaging, or senescence, decreasing the compensatory hypertrophy of the kidney or heart, decreasing the number of hepatic microgranulomas, or reducing aging-related hepatic lipid accumulation, restoring the aging-related changes in intercellular cell-cell communication, reduce tissue fibrosis, or reducing spontaneous tumor incidence in the subject.

7. The method of claim 1, wherein the method is further for restoring aging-related dysregulation.

8. The method of claim 1, wherein the subject is an aged subject.

9. The method of claim 1, wherein the method is further for increasing muscle mass and physical performance.

10. The method of claim 1. wherein the subject is a postmenopausal subject or a healthy subject.

11. The method of claim 1, wherein the method is further for inhibiting multifactorial aging process.
